(19) United States
(12) Patent Application Publication
(10) Pub. No.: US 2003/0175329 A1
(43) Pub. Date: Sep. 18, 2003

(54) Semisolid topical hormonal compositions and methods for treatment

(75) Inventors: Daniel L. Azarnoff, Hillsborough, CA (US); Vivien H.W. Mak, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cellegy Pharmaceuticals, Inc., South San Francisco, CA

(21) Appl. No.: 10/264,850

(22) Filed: Oct. 4, 2002

Related U.S. Application Data
(60) Provisional application No. 60/327,423, filed on Oct. 4, 2001.

(51) Int. Cl. A61K 31/56; A61K 9/70; G06F 19/00; G01N 33/48; G01N 33/50

(52) U.S. Cl. 424/449; 514/177; 514/182; 702/19

(57) ABSTRACT

Semisolid topical pharmaceutical compositions comprising a therapeutically effective amount of a mammalian hormone and an effective amount of a penetration enhancer and methods for their use are provided. The pharmaceutical compositions and methods for their use can provide blood or plasma levels of the administered hormone within a predetermined or normal range of hormone values. In particular embodiments, the hormone is testosterone or estrogen and the amount to be applied to the skin of the subject is determined according to the weight or body mass index of the subject. The topical composition can be formulated in solutions, creams, lotions, ointments, and gels.

### CROSS-REFERENCE TO RELATED APPLICATIONS

This application claims priority from U.S. Patent Application No.60/327,423 filed Oct. 4, 2001 which is incorporated by reference in its entirety.

### FIELD OF THE INVENTION

This invention lies in the technology of the transdermal or topical treatment of human subjects with semisolid topical pharmaceutical compositions comprising therapeutically effective amounts of a mammalian hormone and an effective amount of a skin penetration enhancer. In particular, the present invention provides such pharmaceutical compositions and methods for their administration so as to provide blood or plasma levels of the administered hormone within a predetermined or normal range of hormone values. In particular embodiments, the hormone is testosterone or estrogen and the amount to be applied to the skin of the subject is according to the body weight or body mass index of the subject.

### BACKGROUND OF THE INVENTION

During the past decade, the feasibility of the dermal route for systemic drug delivery has been established for a variety of therapeutic agents. Such transdermal therapeutic systems include those containing scopolamine, glyceryl trinitrate, clonidine, fentanyl, nicotine, testosterone and estradiol. Ultimately, the success of transdermal systems depends both on the ability of the drug to permeate the skin in sufficient quantities and at a sufficient rate to achieve the desired therapeutic effect and on the ability to adjust the dosage so as to increase or decrease the amount absorbed so as to assure an efficacious level is achieved without exceeding the threshold for adverse effects.

Pharmaceutical compositions comprising the mammalian hormone testosterone can be taken as an example of the use of topical compositions for systemic delivery of a hormone. Testosterone is the principal androgen secreted by the testes. It is involved in several developmental and physiological processes, including virilization of the male reproductive tract, skeletal muscle development, growth in stature, male pattern hair growth at onset of puberty, spermatogenesis in adults and control of the gonadotropic functions of the pituitary by down-regulating the synthesis of luteinizing hormone (LH). It also plays a major role in male sexual behavior.

The hormonal regulation of activities in the pituitary-testicular axis involves interactions among the hypothalamus, anterior pituitary, testis and seminiferous tubules. The secretion of gonadotropin releasing hormone (GnRH) by the hypothalamus stimulates the release of follicle stimulating hormone (FSH) and luteinizing hormone (LH) by the pituitary. FSH acts directly on the Sertoli cells within the seminiferous tubules to stimulate the synthesis of an androgen-binding protein. LH induces the Leydig cells to produce testosterone, which diffuses into the adjacent tubules and stimulates spermatogenesis. Testosterone also moderates LH secretion through a feedback loop on the hypothalamus and possibly the anterior pituitary. The Sertoli cells secrete the protein inhibin, which acts on the pituitary to limit FSH secretion. It may also act indirectly via the hypothalamus to limit GnRH, and thereby FSH secretion.

Male hypogonadism is the result of inadequate production of testosterone by the Leydig cells of the testes. The etiology of hypogonadism may be primary or secondary. Primary hypogonadism is associated with dysfunction in the testis. Idiopathic primary testicular failure affects approximately 5% of the male population. Less common causes are Kleinfelter's syndrome, bilateral cryptorchidism, myotonic dystrophy, polyglandular failure, gonadal dysgenesis and vanishing testis syndrome. Testicular irradiation, autoimmune testicular failure and chemotherapeutic testicular changes may also cause testosterone deficiency. Acquired etiologies include surgical or blunt trauma, testicular torsion, and infections.

Secondary hypogonadism is due to inadequate stimulation of a potentially normal testis. The causes may be of hypothalamic or pituitary origin, including GnRH deficiency, isolated FSH or LH deficiencies, acquired gonadotropin deficiencies, prolactin secreting tumors, severe systemic illness, uremia and hemochromatosis.

The testes produce approximately 95% of the normal adult male daily output of 7 mg/24 hours (see, Lipsett, M B, Steriod Secretion by the Human Testis. the Human Testis, p 407 (1970); Odell, W D, et al., Clin Endocrin, 8:149-81 (1978)), the remainder coming from metabolism of adrenal androgens. Testosterone circulating in the blood is bound to sex hormone binding globulin (SHBG) with high affinity (see, Anderson, P C, Clin Endocrin, 3:69-96 (1974)), only 2% being unbound, and to albumin with low affinity. SHBG is high in prepubertal children, declines during puberty and adulthood and increases again during the later decades of life. The albumin bound testosterone easily dissociates and is presumed to be biologically active whereas that bound to SHBG is considered biologically inactive. Bioactive testosterone therefore is considered to be the unbound fraction plus that bound to albumin. The total amount of testosterone and SHBG in serum determines the bioactive moiety. Hypogonadism is reflected by serum levels of testosterone of less than 300 ng/dL, the normal range being 300 to 1140 ng/dL in normal young adult males. Endogenous total testosterone serum concentrations in normal young males follow a diurnal pattern (see, Bremner, W J, et al., J Clin Endocrin Metab, 56:1278-81 (1983); Nieschlag, E, et al, Dtsch Med Wochenschr, 100:1773-4 (1995)). This diurnal rhythm present in early puberty is significantly less noticeable in elderly men (see, Bremner, W J, et al., J Clin Endocrin Metab, 56:1278-81 (1983)). Serum unbound testosterone levels also progressively decrease with age (see, Bremner, W J, et al., J Clin Endocrin Metab, 56:1278-81 (1983)).

Testosterone is rapidly metabolized in human males with half-lives varying from 10 to 100 minutes reported in the literature. No age related effects of testosterone metabolism have been observed in men up to age 65 years. Testosterone is converted to two active metabolites, 5-α dihydrotestosterone (DHT) and 17-β estradiol (E2). The average DHT:T and E2:T ratios in normal men are approximately 1:10 and 1:200, respectively. Following IM injection, about 90% of a testosterone dose is excreted in urine as glucuronide and sulfate conjugates of testosterone and its metabolites. Testosterone is inactivated primarily in the liver. About 6% is excreted mostly unconjugated in the feces. DHT binds to SHBG with greater affinity than testosterone. DHT is further metabolized in reproductive tissues to 3-α and 3-β androstanediol. In many tissues the activity of testosterone appears to depend on its reduction to DHT which binds to cytosol receptor proteins. The steroid receptor complex is then transported to the nucleus where it initiates transcription events and cellular changes related to androgen action.

Males with primary and some with secondary hypogonadism can be treated by administration of testosterone. In addition, it should be noted that estrogen has been reported in a series of recent studies (see, Grodstein, F, et al., New Eng J Med, 336-1769 (see, 1997); Henderson, V W, et al., Psychoneruoendorinology, 21:421-30 (see, 1996)) in females to ameliorate heart disease, improve memory, delay the onset of Alzheimer's disease and prolong life significantly. Since testosterone is the major source of estrogen in males, similar benefits may accrue. In a recent study in 33 healthy young men, individuals with high levels of estradiol performed better on two measures of visual memory than those with normal but lower levels although no correlation was found between individuals with high and low testosterone serum levels (see, Kampen, D L, et al., Behav Neurol, 110:613-7 (1996)).

Several recent studies continue to confirm the physiological and pharmacological effects and safety, even of large doses, of testosterone in hypogonadal and eugonadal men. (see, Morales, A, et al., J Urol, 157:849-54 (see, 1997); Wang, C, et al., J Clin Endocrinol Metab, 81:3578-83 (see, 1996); Bhasin, S, et al., J Clin Endocrinol Metab, 82:407-13 (see, 1997); Brodsky, I G, et al., J Clin Endocrinol Metab, 81:34469-75 (see, 1996); Heikkonen, E, et al., Alcohol Clin Exp Res, 20:711-6 (see, 1996); Gulledge, T P, et al., Eur J Appl Physiol, 73:582-3 (1996); Tiktinskili, O L, et al., Urol Nefrol (Mosk), XX:47-48 (1996); Wu, F C, et al., Fertil Steril, 65:626-36 (1996); Marcovinna, S M, et al., Atherosclerosis, 122:89-95 (1996); Meikle, A W, et al., J Clin endocrin Met, 81:1832-40 (1996); Brocks, D R, et al., J Clin Pharmacol, 36:732-9 (1996); Cooper, C S, et al., J Urol, 156:438-41 (1996))

Oral doses of testosterone undecanoate administered to 23 hypogonadal males for at least 60 days restored serum testosterone levels in all the men, but measurable improvement in sexual attitudes and performance was seen in only 61%. The investigators concluded that testosterone undecanoate is an effective agent for treating hypogonadism and that conventional biochemical hormone determinations lack predictive value and fail to correlate with response (see, Morales, A, et al., J Urol, 157:849-54 (1997)). Mood also improved in hypogonadal men receiving replacement with 200 mg testosterone enanthate IM every 20 days as well as with sublingual administration of 2.5 and 5.0 mg testosterone cyclodextrin three times a day for 60 days (see, Wang, C, et al., J Clin Endocrinol Metab, 81:3578-83 (1996)).

Seven hypogonadal men in a setting of controlled nutrition and exercise were treated for 10 weeks with testosterone enanthate (100 mg/wk) by IM injections. The replacement therapy increased fat-free mass and muscle size and strength in these hypogonadal men. No significant change in hemoglobin, hematocrit and transaminase levels were observed (see, Bhasin, S, et al., J Clin Endocrinol Metab, 82:407-13 (1997)). Testosterone increases skeletal muscle mass by stimulating the rate of muscle protein synthesis (see, Brodsky, I G, et al., J Clin Endocrinol Metab, 81:34469-75 (1996)).

As in experimental animals, alcohol also depresses testosterone secretion in human beings. The alcohol effect is prolonged by physical exertion, which appears to be mainly a consequence of direct inhibition at the testicular level although a role for LH as a contributory regulator could not be totally ruled out (see, Heikkonen, E, et al., Alcohol Clin Exp Res, 20:711-6 (1996)). Endurance trained men have low resting testosterone concentrations without any significant increases in LH concentrations (see, Gulledge, T P, et al., Eur J Appl Physiol, 73:582-3 (1996)).

The safety of testosterone administration is well established. Testosterone may, however, cause the following adverse reactions: gynecomastia, fatigue, priapism, weight gain, decreased high density-lipoprotein cholesterol, increased prostate size and difficulty in urination. Androgens are contraindicated in men with carcinoma of the breast, known or suspected carcinoma of the prostate, and must be used cautiously in men with prostatic hypertrophy.

Doses of testosterone undecanoate as large as 160 mg/day for 10-12 days and 40-80 mg/day for up to six weeks have been administered safely to young and middle-aged males for copulative disorders (see, Tiktinskili, O L, et al., Urol Nefrol (Mosk), XX:47-48 (1996)). In an evaluation of testosterone enanthate (200 mg IM weekly) as a contraceptive in 271 healthy males, aged 21-45 years, the most common adverse events were acne, fatigue and weight gain. Gynecomastia and prostate problems were also observed in only 24 and 9 men respectively. Testosterone enanthate increased body weight, hemoglobin and urea but decreased testicular volume and creatinine. Serum triglyceride, cholesterol and low-density lipoprotein cholesterol were unchanged. High-density lipoprotein cholesterol was decreased 14-18%. Liver transaminases were increased by 36 to 51% in the Chinese subjects, but remained unchanged in non-Chinese subjects. These changes returned to baseline within six months of discontinuing treatment (see, Wu, F C, et al., Fertil Steril, 65:626-36 (1996)).

Although in healthy males a 14-18% decrease in HDL or “good” cholesterol has been reported following administration of testosterone enanthate (see, Wu, F C, et al., Fertil Steril, 65:626-36 (1996)), the same dose had a beneficial effect on elevated concentrations of lipoprotein (a) [Lp(a)] in serum. The latter is associated with an increased incidence of myocardial infarction in men. No correlation was found between the baseline Lp(a) values and the baseline values of testosterone or estradiol. The effect of weekly IM injections on Lp(a) levels in healthy men varied widely and was dependent upon the pretreatment Lp(a) concentration. No significant decrease was observed in the 10 subjects with low Lp(a) values (<25 nmol/L), while there was a significant decrease of 25 to 59% in the 9 subjects with values above 25 nmol/L (see, Marcovinna, S M, et al., Atherosclerosis, 122:89-95 (1996)).

The current products available for administration of testosterone are oral tablets, injectable depot solutions, a topical gel, and two types of transdermal patches, one which is applied to the scrotum and the other to the skin of abdomen, back, thigh or upper arm. Variation in bioavailability of application sites was determined for a permeation-enhanced testosterone transdermal delivery system in 34 hypogonadal men (see, Meikle, A W, et al., J Clin endocrin Met, 81:1832-40 (1996)). Two transdermal delivery systems (Androderm®) were applied to several sites in a sequential cross-over design. Serum concentrations of total testosterone (T), bioavailable testosterone (BT), dihydrotestosterone (DHT) and estradiol (E2) increased from hypogonadal levels into their respective normal physiological ranges and declined to baseline levels within 24 hr. after system removal. Peak concentrations occurred approximately 8 hr. after application for T and BT and 13 hr. for DHT and E2. The half lives of each hormone were: T=1.29±0.71 hr, BT=1.21±0.75 hr, DHT 2.83±0.97 hr and E2=3.53±1.93 hr. Hormone profiles were qualitatively similar at each site of application although the time-average steady state concentrations were statistically highly significantly different. Based on the BT levels, the rank order of the sites was back>thigh>upper arm>abdomen>chest>shin.

In a study using the same patch, the increase in AUC and morning testosterone concentration was proportional to the increase in dose from two to three trasdermal patch systems, but somewhat less for the increase from the one to two patch systems (Brocks, D R, et al., J Clin Pharmacol, 36:732-9 (1996))

Serum T, bioactive testosterone (BT), DHT and E2 levels following daily transdermal delivery have been compared to 200 mg intramuscular testosterone enanthate administered every 2 weeks for 6 months. Transdermal and intramuscular administration maintained serum T (82 vs 72%), BT (87 vs 39%), DHT (76 vs 70%) and E2 (81 vs 35%) levels within the normal range throughout the dosing interval. All but the DHT difference between the two routes of administration were statistically significant. Use of the scrotal testosterone patch results in a 15% difference between Cₘₐₓ and Cₘᵢₙ. This patch delivers 6 mg/day via scrotal skin, a site that is 5 times more permeable than non-genital skin sites.

In clinical studies with the Androderm patch, 93% of patients were treated with two testosterone systems daily; 6% used three and 1% used one. In several open trials that included 94 hypogonadal males, ages 15 to 65 years, average morning testosterone serum concentrations within the normal reference range were found in 92% of the patients; 1% was high and 7% were low.

Twenty nine patients completed 12 months of testosterone transdermal system treatment. In additional to achieving adequate serum levels of T and metabolites, the symptoms of hypogonadism, including but not limited to, fatigue decreased from 79 to 10% and depression (see, Beck Depression Inventory, Brantley P J, Mehan D J Jr., Thomas J L. The Beck Depression Index (BDI) and the Center for Epidemiologic Studies Depression Scale (CES-D in Marhish M E (ed.), Handbook of Physiological Assessment in Primary Care Settings. Lawrence Erlbaum Associates, Inc. Mahwah, N.J. 2000)) decreased from 6.9 to 3.9. The number of self-reported erections increased from 2.3 to 7.8 and in mean duration of erections from 0.23 to 3.9 hours per night, all highly statistically significant. Prostate size and serum prostate specific antigen (PSA) concentrations during treatment were comparable to values reported in eugonadal men. In healthy young men receiving weekly injections of 100, 250, or 500 mg testosterone IM for 15 weeks, significant increases in total and free testosterone but no significant change in serum total and free PSA were detected (see, Cooper, C S, et al., J Urol, 156:438-41 (1996)).

Androgens are important hormones in women that have diverse actions including sexual behavior, affect, cognitive functioning, muscle mass, and maintenance of bone density. The decline in the production of ovarian and adrenal androgens that commences in the decade preceding the average age of naturally occurring menopause may impact significantly on women's health. The clinical sequelae of androgen deficiency in women have only recently been acknowledged, and androgen replacement for symptomatic women is becoming an increasingly important therapeutic concept.

Traditionally, testosterone has been regarded as the male hormone and estrogen as the female hormone, but this mutual exclusivity no longer is valid. Recent studies demonstrate that estrogen and testosterone are involved in both male and female embryologic development (see, McEwen, B. S., In: Notman M T, Nadelson C C, eds. Women and Men: New Perspectives on Gender Differences. Washington D.C.: American Psychiatric Press, 35-41 (1991)) and evidence is emerging, particularly in postmenopausal women, confirming the role of androgens in female sexual behavior, affect, bone, muscle, and cognitive functioning (see, Kaplan, H. S. et al., Sex Marital Ther, 19:3-24 (1993)).

Androgen Physiology in the Premenopausal Years

Androgens are produced both by the ovaries and the adrenals, which synthesize dehydroepiandrosterone (DHEA), androstenedione (A), and testosterone (T). At least 50% of circulating T is produced by peripheral conversion of the androgens to T, with A being the main precursor (see, Kirschner, M. A. et al., Metabolism 21:667-688 (1972)). Only 1-2% of total circulating T is free or biologically active; the rest is bound by sex-hormone binding globulin (SHBG) and albumin. The order of binding affinity for the steroids most strongly bound to SHBG is DHT>T>androstenediol>estradiol>estrone. In women, as in men, alterations in the level of SHBG have dramatic effects on levels of free T, as SHBG binds 99% of the total circulating T (see, Dunn, J. F. et al., J Clin Endocrinol Metab, 53:58-68 (1981)). Increased levels of estradiol (E2) increase SHBG levels, except when it is administered transdermally (see, Basbug, M. et al., Eur J. Obstet Gynecol Reprod Biol, 73(2):149-52 (1997)). Thus high estrogen states such as pregnancy oral contraceptive use and possibly estrogen replacement therapy may result in decreased free T levels, and exacerbate T deficiency symptoms (see, Davis, S. R. et al., J Clin Endocrinol Metab (6):2759-63 (1996)).

Changes in Androgens With Menopause

The effect of the menopausal transition on circulating androgen levels has been addressed in several studies with variable results. Longcope et al. (see, Longcope, C. et al., Maturitas 8:189-196 (1986)) did not observe any change in T, DHT, or A over 80 months after the final menstrual period (FMP), although they noted that the mean concentration of T in all their subjects was significantly less than that in a group of normal young women sampled between days 5 and 7 of their menstrual cycles. Rannevik et al. (see, Rannevik, G. et al., Maturitas 21: 103-113 (1995)) documented a significant decline of about 15% in T and A within the 6 month period following the FMP. SHBG also fell on the order of 15% in association with the FMP; however the ratio of T to SHBG was not affected. In a cross-sectional study of 380 women, aged 46-57 years, Burger et al. (see, Burger, H. G. et al., J. Clin Endocrinol Metab 80:3537-45 (1995)) observed no change in the T:SHBG ratio in relation to menstrual or menopausal status. A decline in T with increasing age has been reported in premenopausal women, such that the levels in women in their forties are approximately 50% of those of women in their twenties (see, Zumoff, B. et al., J Clin Endocrinol Metab 80:1429-30 (1995)). Although the percentage of free T did not vary with age, an absolute decline in free T with age was observed (see, Zumoff, B. et al., J Clin Endocrinol Metab 80:1429-30 (1995)). After ovariectomy, both T and A decrease by about 50% (see, Judd, H. L. Clin Obstet Gynecol 19:775-788 (1976)). After menopause, the primary source of circulating T is from peripheral conversion of androgens secreted by the adrenals (see, Judd, H. L. et al., Am J Obstet Gynecol 118:793-798 (1974); Procope, B. Acta Endocrinol (Copenh) 135:1-86 (1968)).

This decline in total circulating androgens results from ovarian failure and the age-related decline in androgen production. The relative androgen deficiency of women with increasing age and after either natural or surgical menopause may be manifest as impaired sexual function, lessened well-being, loss of energy, and negative effects on bone and muscle mass (see, Sands, R. et al., Am J. Med (Suppl.) 98:765-795 (1995); Frock, J. et al. Psychother Psychosom 57:29-33 (1992); Steinberg, K. K. et al., J Clin Endocrinol Metab 69:533-539 (1989); Hallstrom, T. Clin Obstet Gynecol 4:227-239 (1977)). As the absolute decline in both circulating T and adrenal DHEA production generally commences in the decade preceding menopause (see, Longcope, C. et al., Maturitas 8:189-196 (1986); Zumoff, B. et al., J. Clin Endocrinol Metab 80:1429-30 (1995); Parker, L. N. et al., J. Clin Endocrinol Metab 60:947-952 (1985); Zumoff, B. et al., J. Clin Endocrinol Metab 51:330-334 (1980)), it is not surprising that many women experience the above symptoms in the immediate premenopausal years. This differs from the sudden drop in estrogen levels observed late in the menopausal transition (Burger, H. G. et al., J. Clin Endocrinol Metab 80:3537-45 (1995)). The failure of various studies to demonstrate an association between the menopause and the symptoms attributable to androgen deficiency is probably due to the gradual nature of the androgen decline. Thus these symptoms develop insidiously, in contrast to the more abrupt onset of symptoms of estrogen deficiency (see, Dunn, J. F. et al., J. Clin Endocrinol Metab, 53:58-68 (1981)).

Androgens and Postmenopausal Sexuality

Sexuality and libido are determined by many factors. A strong association between climacteric phase and declining sexual function has been observed by Hallstrom (see, Hallstrom, T. Clin Obstet Gynecol 4:227-239 (1977)). McCoy and Davidson (see, McCoy, N. L. et al., Maturitas 7:203-210 (1985)) prospectively studied the effect of menopause on the sexual experiences and hormonal parameters of a group of women, commencing in the premenopausal years. Women after menopause had significantly fewer sexual thoughts or fantasies, experienced increased lack of vaginal lubrication during intercourse, and were less satisfied. These changes were associated with significant decreases in both estradiol and T, with the decline in T being the most closely associated with lessened coital frequency.

There is increasing agreement that androgens play a key role in female sexuality and that androgen deprivation after menopause contributes to the decline in sexual interest experienced by many women. Controlled studies of the effect of estrogen replacement alone show improvement in vasomotor symptoms, vaginal dryness, and general well-being, but little change in libido (see, Utiah, W. H. S Afr Med J. 46:732-737 (1972); Campell, S. et al., Clin Obstet Gynecol 4:31-47 (1977)). Oral estrogen therapy improves sexual satisfaction in women with atrophic vaginitis causing their dyspareunia, but women without coital discomfort appeared to benefit little or not at all (see, Studd, J. W. W. et al., Br J. Obstet Gynaecol 84:314-315 (1977); Studd, J. W. W. et al., Clin Obstet Gynecol. 4:3-29 (1977)). Exogenous androgen replacement in the form of injected T enanthate enhances parameters of sexual motivation, including the intensity of sexual drive, arousal, and frequency of sexual fantasies in hysterectomized and oophorectomized women over and above the effect achieved with estrogen replacement alone (see, Sherwin, B. B. et al., Psychosom Med 47:339-351 (1985)). Several other investigators have reported improved libido in postmenopausal women treated with subcutaneous T implants in combination with estradiol implant therapy (see, Studd, J. W. W. et al., Br J. Obstet Gynaecol 84:314-315 (1977); Sherwin, B. B. et al., Psychosom Med 47:339-351 (1985); Studd, J. W. W. et al., Clin Obstet Gynecol. 4:3-29 (1977); Burger, H. G. et al., Maturitas. 6:351-358 (1984)). Davis et al. (see, Davis, S. R. et al., Maturitas 21:227-236 (1995)) investigated the effects of subcutaneous T implants on several parameters of sexuality in postmenopausal women in a two-year single blind randomized study. All parameters of sexuality improved with both E implants alone and E combined with T implants; however the inclusion of T resulted in a significantly greater increase in sexual activity, satisfaction, pleasure, orgasm, and relevancy. T administration did not adversely affect blood lipids in that total cholesterol and low-density lipoprotein (LDL) cholesterol fell equally in both groups. The authors concluded that T administration to postmenopausal women enhances sexuality and can be of considerable benefit to women experiencing low libido despite adequate estrogen replacement. Sherwin (see, Sherwin, B. B. et al., Psychosom Med 47:339-351 (1985)) observed a greater effect on sexuality with combined estrogen and T replacement despite their estrogen only group being treated with higher doses of estrogen and achieving higher circulating estrone and estradiol (E2) levels. Similarly, the supraphysiological E2 levels achieved with E2 implants alone do not result in a positive effect on sexuality equivalent to that observed with the addition of T implants (see, Davis, S. R. et al., Maturitas 21:227-236 (1995)). To achieve a good therapeutic response in terms of enhanced libido with postmenopausal androgen replacement, it appears that T levels often need to be restored to at least the upper end of the normal physiological range for young ovulating females (see, Dunn, J. F. et al., J. Clin Endocrinol Metab, 53:58-68 (1981)).

The role of androgen replacement in restoring sexuality after the menopause is significant. Young women who suffer either premature menopause or undergo bilateral oophorectomy early in life frequently experience great distress from their loss of libido. Not only are such women very responsive to androgen replacement in terms of the restoration of their sexuality, but they also frequently experience an enhanced general sense of well being (see, Dunn, J. F. et al., J. Clin Endocrinol Metab, 53:58-68 (1981)).

Relationship Between Androgens and Bone Loss After Menopause

Androgenic steroids are known to be important in the maintenance of bone mass in both men and women (see, Dunn, J. F. et al., J. Clin Endocrinol Metab, 53:58-68 (1981)). Nilas and Christiansen (see, Nilas, L. et al., J. Clin Endocrinol Metab. 65:697-699 (1987)) performed a cross-sectional analysis of the sex hormone concentrations and bone mineral densities of women recruited for a prospective study of risk factors for osteoporosis. After controlling for body weight, a significant negative correlation between SHBG and bone mineral density (BMD) and a significant positive correlation between percent free T and BMD, but no relationship between BMD and E2 was observed in the premenopausal women.

Human osteoblastic cells have been shown to possess androgen receptors (see, Colvard, D. S. et al., Proc Natl Acad Sci USA. 86:854-857 (1989)) and androgens directly stimulate human bone cell proliferation and differentiation (see, Kasperk, C. H. et al., Endocrinology 124:1576-1578 (1989)) and thus may enhance bone formation. In postmenopausal women estrogen acts as an antiresorptive agent on bone, thus limiting bone loss (see, Dunn, J. F. et al., J. Clin Endocrinol Metab, 53:58-68 (1981)).

Ralston et al. (see, Ralston, S. H. et al., Maturitas. 6:341-345 (1984)) investigated the effects of subcutaneous estrogen implants, either alone or with T, on several parameters of calcium metabolism in postmenopausal women. Significant reductions in serum calcium and phosphate, the renal phosphate threshold, and the urinary calcium/phosphate ratio were observed, with no additional benefit of T on those parameters. Raisz et al. (see, Raisz, L. G. et al., J. Clin Endocrinol Metab. 81:37-4 (1995)) compared the effects of estrogen given alone to those of estrogen plus androgen therapy on biochemical markers of bone formation and resorption in postmenopausal women. Urinary excretion of markers of bone formation and resorption decreased equally in both groups. The estrogen only group had a reduction in serum markers of bone formation, whereas in women treated with combined estrogen plus T, all markers of bone formation increased. Treatment with nandrolone decanoate has been shown to increase vertebral BMD in postmenopausal women and has been used for many years to treat postmenopausal osteoporosis (see, Need, G. A. et al., Arch Intem Med. 149:57-60 (1989)). Combined E2 and T replacement with subcutaneous implant pellets increases bone mass in postmenopausal women (see, Savvas, M. et al., Br Med J. 297:331-333 (1988); Savvas, M. et al., Br J. Obstet Gynecol 99:757-760, (1992)) with the effect being significantly greater than that observed using E2 implants alone (see, Davis, S. R. et al., Maturitas 21:227-236 (1995)).

Davis and colleagues (see, Davis, S. R. et al., Maturitas 21:227-236 (1995)) investigated the role of androgens in increasing bone density and improving decreased libido in postmenopausal women in a two-year prospective, randomized trial. Thirty-four postmenopausal volunteers were randomized to either estradiol (50 mg) implants alone (E) or estradiol (50 mg) plus testosterone (50 mg) (E+T). Bone density of total body, lumbar vertebrae (L1-L4) and hip area increased significantly in both treatment groups. Bone density increased more rapidly in the testosterone treated group at all sites. A substantially greater bone density resulted in the E+T group for total body, vertebral, and trochanteric measurements. Total cholesterol and LDL-cholesterol fell in both groups as did total body fat. Total body fat-free mass increased in the E+T group only. The authors commented that the favorable estrogenic effects on lipids were preserved in women treated with testosterone, in association with beneficial changes in body composition.

Watts et al. (see, Watts, N. B. et al., Obstet Gynecol 85(4) 529-537 April (1995)) compared an oral estrogen-androgen combination with estrogen alone on bone, menopausal symptoms, and lipoprotein profiles in postmenopausal women. Surgically induced menopausal women received oral esterified estrogens (1.25 mg) or esterified estrogens (1.25) plus methyltestosterone (2.5 mg) daily for 2 years. Both treatment regimens prevented bone loss at the spine and hip, and combined estrogen-androgen therapy was associated with a significant increase in spinal bone mineral density compared with baseline. In the estrogen group, HDL cholesterol increased significantly and LDL-cholesterol decreased significantly, and in the estrogen-androgen group cholesterol, HDL-cholesterol and triglycerides decreased significantly. Menopausal symptoms of somatic origin (hot flashes, vaginal dryness, and insomnia) were improved significantly by both treatments. A study by Savvas et al. (see, Savvas, M. et al., Br J. Obstet Gynecol 99:757-760, (1992)) found that subcutaneous implants of estradiol and testosterone result in an increase in bone mass even after many years of oral estrogen replacement therapy. They studied twenty women and investigated bone density of the skeleton after changing from an oral estrogen (1.25 mg) to subcutaneous estradiol (50 mg) and testosterone (50 mg) replacement. Bone density increased significantly by 5.7% at the spine and by 5.2% at the neck of the femur in those women who changed to subcutaneous therapy but remained unchanged in women who remained on oral therapy.

Not all studies have been able to conclusively demonstrate that testosterone confers a beneficial effect greater than estrogen alone. Garnett et al. (see, Garnett, T. et al., Obstet Gynecol 79(6):968-72 June (1992)) studied 50 postmenopausal women who were randomly allocated to receive estradiol alone (75 mg) or estradiol (75 mg) with testosterone (100 mg) every 6 months for 1 year. After one year, the authors reported that women receiving either treatment had significant increases in bone density at both the lumbar spine and neck of femur, and that this increase in bone density was correlated significantly with serum estradiol levels attained, but unrelated to chronological age, menopausal age, or initial bone density. The study however, could not demonstrate that testosterone conferred an additional bone sparing effect in postmenopausal women.

Based on the data that has been accumulated on the treatment of female androgen deficiency with subcutaneous testosterone implants, it is evident that maintaining the serum bioactive testosterone concentration in the normal physiological range (1.1-14.5 ng/dL), preferrably in the upper one-third of the normal physiological range (8-14.5 ng/dL), should be effective in restoring most postmenopausal females to sexually and physically normal states. Osteoporosis, once thought to be uncommon in males, is being increasingly diagnosed. The incidence of osteoporosis in males living in Rochester, Minn. was reported to be 73/100,000 person years (Cooper C, Atkinson E J, O'Fallon M, Melton L J III. Incidence of Clinically Diagnosed Vertebral Fractures: A Population-Based Study in Rochester, Minn., 1985-1989. J Bone Min Res 1992;7(2):221-227). In a recent study of 114 men cohort, osteoporosis was most commonly due to hypogonadism (Tordjman K M, Weisman Y, Osher E, Greenman Y, Shenkerman G. Male Osteoporosis Is Most Commonly Due to Hypgonadism: Analysis of a Cohort of 114 Men. Poster Sessions. The Endocrine Society. P3-). Testosterone deficiency is associated with progressive loss of bone mass. Transdermal testosterone therapy has demonstrated efficacy on improving bone mineral density of hypogonadal men.

Osteoporosis, once thought to be uncommon in males, is being increasingly diagnosed. The incidence of osteoporosis in males living in Rochester, Minn. was reported to be 73/100,000 person years (Cooper C, Atkinson E J, O'Fallon M, Melton L J III. Incidence of Clinically Diagnosed Vertebral Fractures: A Population-Based Study in Rochester, Minn., 1985-1989. J Bone Min Res 1992;7(2):221-227). In a recent study of 114 men cohort, osteoporosis was most commonly due to hypogonadism (Tordjman K M, Weisman Y, Osher E, Greenman Y, Shenkerman G. Male Osteoporosis Is Most Commonly Due to Hypgonadism: Analysis of a Cohort of 114 Men. Poster Sessions. The Endocrine Society. P3-). Testosterone deficiency is associated with progressive loss of bone mass. Transdermal testosterone therapy has demonstrated efficacy on improving bone mineral density of hypogonadal men.

Androgens effects on cognitive function are domain-specific. The observations that men outperform women in a variety of visuo-spatial skills suggest that androgens enhance visuospatial skills (Maccoby E E J C. The Psychology of Sex Differences. Stanford, Calif.: Stanford University Press, 1974). Janowsky et al. (Janowsky J S, Oviatt S K, Orwoll E S. Testosterone influences spatial cognition in older men. Behav Neurosci 1994; 108:325-32) tested verbal and visual memory, spatial cognition, motor speed and cognitive flexibility in a group of healthy older men who received 3 months of testosterone replacement. Testosterone replacement was associated with a significant improvement in spatial cognition only. Serum testosterone levels were not significantly correlated with spatial performance, but estradiol levels showed a significant inverse relationship with spatial performance suggesting that estradiol might inhibit spatial ability. In Kung San Bushmen of Southern Africa (Barrett-Connor E, Goodman-Gruen D, Patay B. Endogenous sex hormones and cognitive function in older men. J Clin Endocrinol Metab 1999; 84:3681-5), testosterone, but not estradiol, levels correlated with better spatial ability and with worse verbal fluency. Circulating levels of dihydrotestosterone, a metabolite of testosterone that is not converted to estrogen, positively correlated with verbal fluency. Barrett-Connor, et al ( Barrett-Connor E, Goodman-Gruen D, Patay B. Endogenous sex hormones and cognitive function in older men. J Clin Endocrinol Metab 1999; 84:3681-5; Barrett-Connor E, Goodman-Gruen D. Cognitive function and endogenous sex hormones in older women. J Am Geriatr Soc 1999; 47:1289-93.) found positive associations between total and bioavailable testosterone levels, and global cognitive functioning and mental control, but not with visuospatial skills. Other studies (Gouchie C, Kimura D. The relationship between testosterone levels and cognitive ability patterns. Psychoneuroendocrinology 1991; 16:323-34; Shute V J P J, Hubert L, Reynolds R W. The relationship between androgen levels and human spatial abilities. Bull Psychonomic Soc 1983; 21:465-468) have reported a curvilinear relationship between androgen levels and spatial ability such that females with high testosterone levels and males with low testosterone levels show the best performance. Women with congenital adrenal hyperplasia with high androgen levels score higher on tests of spatial cognition than their age- and gender-matched siblings. On the contrary, 46 XY individuals with androgen insensitivity syndrome perform worse on tests of spatial cognition than their age-matched male siblings.

Turner's syndrome is a genetic disorder, specific to women, in which one of the X chromosomes is partially or completely deleted. This syndrome is associated with physical features such as short stature or failure in primary and secondary sexual development, together with a specific pattern of cognitive functions (Cornoldi C et al. Visuospatial working memory in Turner's syndrome. Brain Cogn. 2001 June-July;46(1-2):90-4.) Other problems can include a webbed neck, heart defects, kidney abnormalities, learning difficulties, skeletal abnormalities, hearing loss, liver dysfunction, infertility, and thyroid dysfunction. Normally, females have two X chromosomes. In some cases of Turner's Syndrome, however, one X chromosome is missing from the cells (45,X); research studies suggest that approximately 40 percent of these individuals may have some Y chromosomal material in addition to the one X chromosome. In other affected females, both X chromosomes may be present, but one may have genetic defects. In still other cases, some cells may have the normal pair of X chromosomes while other cells do not (45,X/46,XX mosaicism). Although the exact cause of Turner's Syndrome is not known, it is believed that the disorder may result from an error during the division (meiosis) of a parent's sex cells. (Conway G S. The impact and management of Turner's syndrome in adult life. Best Pract Res Clin Endocrinol Metab 2002 June;16(2):243-61)

Children with Turner's Syndrome are not usually growth-hormone deficient, but they do increase their rate of growth with the addition of human growth-hormone therapy. Recent studies indicate that much of the growth deficit in children with Turner's Syndrome can be restored by injections of human growth hormone before growth is completed.

Turner's syndrome (TS) has a characteristic neurocognitive profile. It has been suggested that women affected by Turner's syndrome perform poorly in tasks measuring visuospatial abilities and have a verbal IQ significantly higher than performance IQ. Although this result has received strong empirical support, the nature of the visuospatial deficit is still unclear. Recent studies on visuospatial processes have highlighted that the underlying cognitive structure is more complex than previously suggested and several dissociations have been reported (e.g., visual vs spatial, sequential vs simultaneous, or passive vs active processes). Verbal abilities are, in general, normal; however, women with TS, as a group, have specific deficits in visual-spatial abilities, visual-perceptual abilities, motor function, nonverbal memory, executive function, and attentional abilities. Observed deficits could be caused by genetic or endocrine factors. Similar to children and adolescents with TS, adults with TS have normal verbal IQ but have relative difficulty on measures of spatial/perceptual skills, visual-motor integration, affect recognition, visual memory, attention, and executive function despite estrogen replacement. These deficits are apparent in women with TS despite apparently adequate estrogen effect, either endogenous or by hormone replacement (Ross J L et al Persistent cognitive deficits in adult women with Turner syndrome. Neurology. 2002 January 22;58(2):218-25.). Since testosterone is related to the superior visual cognitive function in men, it follows that androgen replacement therapy could provide significant benefit to TS subjects.

Current research is assessing the best way to administer female sex hormones to young girls who need this therapy to promote pubertal development and achieve sexual maturity. Additionally, these hormones may promote maximum bone development and growth in adolescents. In addition to treatment with growth hormones and sex hormones, thyroid hormone is important for growth and health in girls who suffer from thyroid dysfunction. (Perheentupa J, Lenko H L, Nevalainen I, Niittymaki M, Soderholm A, Taipale V. Hormonal treatment of Turner's syndrome. Acta Paediatr Scand Suppl 1975;256:24-5)

Adults with Turner's syndrome are susceptible to a range of disorders such as osteoporosis, hypothyroidism, high blood pressure, high cholesterol and diabetes. Treatment with estrogen, progestin and testosterone may increase sex drive, energy and overall sense of well being in these women. (Conway G S. The impact and management of Turner's syndrome in adult life. Best Pract Res Clin Endocrinol Metab 2002 June;16(2):243-61)

Chronic Fatigue Syndrome/Chronic Fatigue Immune Dysfunction Syndrome is considered a medically unexplained condition affecting both men and women characterized by disabling fatigue accompanied by infectious, rheumatological, and neuropsychiatric symptoms. In general, a patient is diagnosed as having chronic fatigue syndrome if he satisfies the following two criteria:

1. Has severe chronic fatigue of six months or longer duration with other known medical conditions excluded by clinical diagnosis, and

2. Concurrently has four or more of the following symptoms: substantial impairment in short-term memory or concentration, sore throat, tender lymph nodes, muscle pain, multi-joint pain without swelling or redness, headaches of a new type, pattern or severity, unrefreshing sleep, and post-exertional malaise lasting more than 24 hours.

The exact cause or causes of Chronic Fatigue Syndrome remain unknown. One of the first causes proposed for CFS was low adrenal function. More recently, possible causes have been proposed to be Epstein-Barr virus, cytomegalovirus (CMV), herpes simplex I and II, or Herpes VI. But treatment with anti-viral medications does not necessarily relieve the symptoms experienced by the patient. Although the ailment can occur after severe infection, some researchers believe chronic fatigue syndrome is an auto-immune disease, while other researchers contend little data exists to support theories of an infectious or immunologic process in disease maintenance.

Other possible pathophysiological processes proposed as causes include a covert encephalopathy, impaired physiological capability to respond to physical and mental stressors, and psychological factors related to concerns about effort exacerbating symptoms. In addition, some data do exist to indicate that environmental agents and toxins also can elicit a state of chronic fatigue.

Accumulating data support the theory that CFS has multiple causes and may represent a common endpoint of disease resulting from multiple precipiating causes. (Natelson B H, Lange G. A status report on chronic fatigue syndrome. Environ Health Perspect 2002 August;110 Suppl 4:673-7) Human growth hormone, melatonin, serotonin, tryptophan, dehydroepiandrosterone, cortisol, thyroid, testosterone, estrogen, and progesterone have all been used to treat Chronic Fatigue Syndrome and are considered to be useful by some clinicians.

Epstein-Barr virus, frequently referred to as EBV, is a member of the herpes virus family and one of the most common human viruses. Epstein-Barr virus infects and persists for life in the majority of the human population. Many children become infected with EBV, and these infections usually cause no symptoms or are indistinguishable from the other mild, brief illnesses of childhood. When infection with EBV occurs during adolescence or young adulthood, it causes infectious mononucleosis 35% to 50% of the time. (Levitsky V, Masucci M. Manipulation of immune responses by Epstein-Barr virus. Virus Res 2002 September;88(1-2):71)

Symptoms of infectious mononucleosis are fever, sore throat, and swollen lymph glands. Sometimes, a swollen spleen or liver involvement may develop. Although the symptoms of infectious mononucleosis usually resolve in 1 or 2 months, EBV remains dormant or latent in a few cells in the throat and blood for the rest of the person's life. Periodically, the virus can reactivate and is commonly found in the saliva of infected persons. This reactivation usually occurs without symptoms of illness. EBV also establishes a lifelong dormant infection in some cells of the body's immune system. (Rickinson A. Epstein-Barr virus. Virus Res 2002 January 30;82(1-2):109-13)

There is no specific treatment for infectious mononucleosis, other than treating the symptoms. No antiviral drugs or vaccines are available. Treatment with steroids is sometimes prescribed to control the swelling of the throat and tonsils. The use of steroids has also been reported to decrease the overall length and severity of illness. Symptoms related to infectious mononucleosis caused by EBV infection seldom last for more than 4 months. When such an illness lasts more than 6 months, it is frequently called chronic EBV infection. However, valid laboratory evidence for continued active EBV infection is seldom found in these patients. The illness should be investigated further to determine if it meets the criteria for chronic fatigue syndrome.

In addition to the diseases and disorders discussed above, a number of systemic disorders may suppress testosterone levels, including hepatic cirrhosis, chronic renal failure, sickle cell anemia, thalassemia, hemochromatosis, AIDs virus, amyloidosis, chronic obstructive pulmonary disease (COPD), rheumatoid arthritis, chrnic infection, and inflammatory or debilitating conditions. A number of drugs may affect testosterone levels. Drugs that are known to decrease testosterone levels include GnRH agonists, and antagonists, estrogens, progestins, glucocorticoids, ketoconazole, aldactone, thiazide diuretics, opiates, anabolic steroids, amiodarone, and a number of psychotropic agents. Agents that impair testosterone action at the receptor level include aldactone, cimetidine, flutamide, and other androgen antagonists.

The oral route of testosterone administration is associated with an increased incidence of liver disease, including cancer. Most patients find the depot solutions objectionable due to the need for relatively frequent injections and the pain that accompanies deep muscle injections, and dislike the patches, especially the non-scrotal patch because of a high incidence of inflammation at the site of application.

Traditionally, transdermal devices or patches have been employed when a specific controlled input kinetic function was desired for the topical or transdermal delivery of an active agent. Methods of preparing patches and transdermal devices capable of producing reproducible controlled input kinetic functions are well known in the art. However, such patches and devices, as exemplied above for testosterone, are often associated with skin irritation which can adversely affect compliance with a prescribed therapeutic regimen.

Methods of preparing semisolid dosage forms (e.g., gels, ointments) capable of producing reproducible controlled input kinetic functions have not been known. This is especially true for semisolid dosage forms currently known in the art, because the skin itself has been the major factor controlling delivery of the active, as opposed to formulation and device components controlling delivery of the active, for example, with patch-type devices. Semisolid dosage forms offer many advantages over patch-type devices: they typically are less irritating to the skin, are not visible once applied, are easily applied to many areas of the body, offer easy dose titration, and are generally more acceptable to the patient. However, the use of semisolid dosage forms to topically or transdermally deliver active agents, including, but not limited to, testosterone, has up-to-now, been limited by their dependence on the patient's skin to control the input kinetic function for the active agent, and by the variability in delivery between patients that results.

With respect to semisolid topical pharmaceutical compositions, WO 02/17926 discloses, for instance, a topical testosterone composition for treating hypogonadism where a 50% increase in the initially applied daily 5 g dose provided no additional increase, if any, in the blood C_{avg} testosterone level at steady state for the subpopulation of subjects whose testosterone levels were insufficiently raised at the 7.5 g dose level after an initial daily 5 g dose level had failed. There is a need for methods by which to exert control of the active agent input kinetic function for semisolid dosage forms that are not dependent entirely on the skin, and that offer the potential for reducing input kinetic variability between patients so as to finally obtain a substantial dose response relationship between the amounts of a semisolid topical composition applied to an individual patient or subject and the observed therapeutic measure (e.g., serum hormone level, functional response) of the individual patient.

The present invention fulfills these and other needs. It provides topical semisolid topical pharmaceutical compositions, methods of treatment therewith, and methods of dosing that allow a better dose titration of the subject so treated.

### SUMMARY OF THE INVENTION

In one aspect, the present invention provides a method for determining the initial amount or dose of a topical semisolid pharmaceutical composition comprising a therapeutic amount or concentration of a mammalian hormone and an effective amount or concentration of a penetration enhancer to administer to the skin of a human subject. In this aspect, the initial amount or dose of the composition is based upon an empirically determined relationship between such parameters as bodyweight, subcutaneous fat thickness, and Body Mass Index (BMI); the amounts or dosages applied to a relevant subject population; and the resulting measured serum testosterone levels at steady state. The applied amount or dose of the composition will generally be from 0.1 to 10 g.

In a second aspect, the invention provides a method of treating a human subject by applying an initial amount or dose of a topical semisolid pharmaceutical composition comprising a therapeutic amount or concentration of the mammalian hormone and an effective amount or concentration of a penetration enhancer to the skin of the subject in an amount determined according to an empirically determined relationship between subcutaneous fat thickness, body weight, or BMI; the amounts or dosages applied to a relevant subject population; and their resulting serum hormone levels at steady state or after a predetermined period of time. In this aspect, a second or subsequent amount or dosage of the composition is thereafter determined after measuring the serum hormone levels of the treated subject at steady state or after the the predetermined period of time and adjusting the dosage upward or downward according to whether the steady state serum hormone levels are above or below a predetermined or targeted range of serum hormone values (e.g., the normal or therapeutically efficacious range for such a hormone).

In a third aspect, the invention provides a metered dose pump set to deliver a daily initial amount or dose of such a topical semisolid pharmaceutical composition. The pump allows various settings and can be set to deliver a fixed amount according to the bodyweight, subcutaneous fat thickness, or BMI of the individual subject as described above. In one embodiment, the pump once set to deliver only the fixed amount can not be reset to deliver another fixed amount.

In some embodiments for each of the first, second and third aspects, the mammalian hormone, hormone agonist, or hormone antagonist includes, but is not limited to the hormone derivative, the hormone metabolite, and the hormone mimetic, or a combination thereof.

In some embodiments in each of the first, second and third aspects of the invention, the mammalian hormone or the mammalian receptor agonist or antagonist is an adrenocortical steroid, or derivative, synthetic analog, mimetic, metabolite, or combination thereof. Examples include, but are not limited to alclometasone diproprionate, amcinonide, beclomethasone diproprionate, betamethasone, betamethasone benzoate, betamethasone diproprionate, betamethasone sodium phosphate, betamethasone valerate, clobetasol proprionate, clocortolone pivalate, cortisol (hydrocortisone), cortisol acetate, cortisol cypionate, cortisol sodium phosphate, cortisol sodium succinate, cortisol valerate, cortisone, cortisone acetate, desonide, desoximetasone, dexamethasone, dexamethasone acetate, dexamethasone sodium phosphate, diflorasone diacetate, fludrocortisone, fludrocortisone acetate, flunisolide, fluocinolone acetonide, fluorometholone, flurandenolide, halcinonide, medrysone, 6α-methylprednisone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and the pharmaceutically acceptable salts, esters, derivatives, mimetics, metabolites, and synthetic analogs thereof.

In some embodiments in each of the first, second and third aspects of the invention, the mammalian hormone or the mammalian receptor agonist or antagonist is a mammalian sex hormone, or salt, ester, derivative, agonist, antagonist, metabolite, mimetic, synthetic analog,.or combination thereof. Examples include, but are not limited to androgen (e.g., androsterone, testosterone, testosterone proprionate, testosterone enanthae, testosterone cypionate, danazol, fluoxymesterone, methyltestosterone, oxandrolone, dihydrotestosterone, methenolone acetate, testosterone undecanoate); estrogen (e.g., estradiol, estradiol valerate, estradiol cyprionate, ethinyl estradiol, mestranol, quinestrol, estrone, estrone sulfate, equilin, conjugated estrogens, diethylstilbestrol); and progestin (e.g., progesterone, hydroxyprogesterone caproate, medroxyprogesterone acetate, 19-nortestosterone, norethynodrel, norgestrel, desogestrel, norgestimate, norethindrone (norlutin), norethindrone acetate (norlutate, aygestin) and the pharmaceutically acceptable salts, esters, derivatives, metabolites, mimetics, or synthetic analogs thereof.

In some embodiments for each of the first, second and third aspects of the present invention the mammalian hormone is an estrogen-like compound. Estrogen-like compounds include those compounds that bind to the estrogen receptor and act as agonists thereof. Estrogen-like compounds include, but are not limited to, 17-β-estradiol, estrone, mestranol, estradiol valerate, estradiol dypionate, ethynyl estrodil, quinestrol, estrone sulfate, phytoestrogens, including, but not limited to, flavones, isoflavones (e.g., genistein), resveratrol, coumestan derivatives, other synthetic estrogenic compounds including pesticides (e.g., p,p′-DDT), plasticizers (e.g., bisphenol A), and a variety of other industrial chemicals (e.g., polychlorinated biphenyls) and the pharmaceutically acceptable salts, esters, derivatives, metabolites, mimetics, or synthetic analogs thereof.

In some embodiments for each of the first, second and third aspects of the present invention the mammalian hormone is a testosterone-like compound. Such testosterone-like compounds include those compounds that bind to the testosterone receptor and act as agonists thereof. Testosterone-like compounds include, but are not limited to, testosterone, testosterone propionate, testosterone enanthate, testosterone cypionate, testosterone undecenoate, dihydrotestosterone, danazol, fluoxymesterone, methyltestosterone, oxandrolone, DHEA and tibolone and the pharmaceutically acceptable salts, esters, derivatives, metabolites, mimetics, or synthetic analogs thereof or mixtures thereof.

In some embodiments for each of the first, second and third aspects of the present invention the mammalian hormone is a progestin-like compound (e.g., progesterone, hydroxyprogesterone caproate, medroxyprogesterone acetate, 19-nortestosterone, norethynodrel, norgestrel, desogestrel, norgestimate, norethindrone (norlutin), norethindrone acetate (norlutate, aygestin)) and the pharmaceutically acceptable salts, esters, derivatives, metabolites, mimetics, or synthetic analogs thereof.

In some embodiments for each of the first, second and third aspects of the present invention, the amount of the composition to be applied to the skin of the subject is determined from the height and weight of the subject. In some such embodiments, the amount is determined according to the Body Mass Index of the subject [e.g., (subject body weight)/(subject body height)² when the weight is expressed in kilograms and the height is expressed in meters] of the subject. In some such embodiments, the dose to be administered increases by an average of about 1-10% for each single unit increase in the BMI for subjects having a BMI value of 25 to 50. In some such embodiments, the subject has a BMI value of at least about 30, 35, 40 or 45. In some such embodiments, the subject is a male with a body weight of at least about 250 pounds, 300 pounds, 350 pounds or 400 pounds. In some such embodiments, the subject is a female with a body weight of at least about 200 pounds, 250 pounds, 300 pounds, or 350 pounds.

In some embodiments for each of the first, second and third aspects of the present invention, the subject is a human male with primary or secondary hypogonadism, AIDS, wasting syndromes associated with chronic illnesses (e.g., AIDS, cancer, cardiovascular disordes, anorexia nervosa), end stage renal disease, chronic fatigue syndrome, Epstein-Barr virus, heart disease, cancer, diabetes, Alzheimer's disease, systemic lupus erythematosus, rheumatoid arthritis, multiple sclerosis, or osteoporosis and the hormone is a testosterone-like compound including but not limited to, testosterone, testosterone propionate, testosterone enanthate, testosterone cypionate, testosterone undecenoate, dihydrotestosterone, danazol, fluoxymesterone, methyltestosterone, oxandrolone, DHEA and tibolone and the pharmaceutically acceptable salts, esters, derivatives, metabolites, mimetics, or synthetic analogs thereof.

In some embodiments for each of the first, second and third aspects of the present invention, the subject is a human female with sexual dysfunction, AIDS, wasting syndromes associated with chronic illnesses (e.g., AIDS, cancer, cardiovascular disordes, anorexia nervosa), end stage renal disease, Turner's Syndrome, chronic fatigue syndrome, Epstein-Barr virus, , heart disease, cancer, diabetes, Alzheimer's disease, systemic lupus erythematosus, rheumatoid arthritis, multiple sclerosis, osteoporosis or with a reduced feeling of well-being and the hormone is i) a testosterone-like compound including but not limited to, testosterone, testosterone propionate, testosterone enanthate, testosterone cypionate, testosterone undecenoate, dihydrotestosterone, danazol, fluoxymesterone, methyltestosterone, oxandrolone, DHEA and tibolone and the pharmaceutically acceptable salts, esters and derivatives, metabolites, mimetics, or systemic analogs thereof. 2) an estrogen like compound, including but not not limited to, 17-β-estradiol, estrone, mestranol, estradiol valerate, estradiol dypionate, ethynyl estrodil, quinestrol, estrone sulfate, phytoestrogens, including but not limited to, flavones, isoflavones (e.g., genistein), resveratrol, coumestan derivatives, and the pharmaceutically acceptable salts, esters and derivatives thereof; iii) a progestin-like compound (e.g., progesterone, hydroxyprogesterone caproate, medroxyprogesterone acetate, 19-nortestosterone, norethynodrel, norgestrel, desogestrel, norgestimate, norethindrone (norlutin), norethindrone acetate (norlutate, aygestin)) or the pharmaceutically acceptable salts, esters derivatives, metabolites, mimetics, or synthetic analogs thereof; or iv) the mixtures thereof.

In some embodiments for each of the first, second and third aspects of the present invention, the penetration enhancer is oleic acid or the alcohol or a pharmaceutical acceptable salt or ester of oleic acid.

In some embodiments for each of the first, second and third aspects of the present invention, the topical composition has a pH value of between about 4 to about 8 and comprises a) the hormone in a concentration of about 0.1% to about 2%, w/w (weight to weight) and b) a penetration-enhancing system consisting essentially of (i) a membrane fluidizer comprising oleic acid; (ii) a C₁-C₄ alcohol; (iii) a glycol, and (iv) optionally a gelling agent. In further such embodiments, the amount of the composition to be applied to the skin of the subject is determined from the height and weight of the subject (e.g., the BMI). In still further such embodiments, the hormone is a testosterone-like compound, estrogen-like compound, progestin-like compound, adrenocorticoid, glucocorticoid or mineralcorticoid and the pharmaceutically acceptable biologically active salts, esters, derivatives, metabolites, mimetics, or synthetic analogs thereof. In some such embodiments where the subject is a human male with primary or secondary hypogonadism, the hormone is a testosterone-like compound including but not limited to, testosterone, testosterone propionate, testosterone enanthate, testosterone cypionate, testosterone undecenoate, dihydrotestosterone, danazol, fluoxymesterone, methyltestosterone, oxandrolone, DHEA and tibolone and the pharmaceutically acceptable salts, esters derivatives, metabolites, mimetics, or synthetic analogs thereof. In some such embodiments where the subject is a human female with sexual dysfunction or with a reduced feeling of well-being or osteoporosis the hormone is i) a testosterone-like compound including but not limited to, testosterone, testosterone propionate, testosterone enanthate, testosterone cypionate, testosterone undecenoate, dihydrotestosterone, danazol, fluoxymesterone, methyltestosterone, oxandrolone, DHEA and tibolone and the pharmaceutically acceptable salts, esters derivatives, metabolites, mimetics, or synthetic analogs thereof; 2) an estrogen like compound, including but not not limited to, 17-β-estradiol, estrone, mestranol, estradiol valerate, estradiol dypionate, ethynyl estrodil, quinestrol, estrone sulfate, phytoestrogens including, but not limited to, flavones, isoflavones (e.g., genistein), resveratrol, coumestan derivatives, and the pharmaceutically acceptable salts, esters, derivatives, metabolites, mimetics, or synthetic analogs thereof; iii) a progestin-like compound, (e.g., progesterone, hydroxyprogesterone caproate, medroxyprogesterone acetate, 19-nortestosterone, norethynodrel, norgestrel, desogestrel, norgestimate, norethindrone (norlutin), norethindrone acetate (norlutate, aygestin)) or the pharmaceutically acceptable salts, esters, derivatives, metabolites, mimetics, or synthetic analogs thereof; or iv) the mixtures thereof.

In some embodiments for each of the first, second and third aspects of the present invention, the pharmaceutical composition has a hormone concentration of about 0.01% to about 5% w/w. In further such embodiments, the hormone is testosterone-like, estrogen-like, progestin-like, a mineral corticoid, glucocorticoids or adrenocorticoid.

In some embodiments for each of the first, second and third aspects of the present invention, the dose is a daily dosage amount; in other embodiments the dose is an individual or single dose amount to be administered one or more times over the course of the day.

In one embodiment, the invention provides a method of administering a therapeutically effective amount of a mammalian hormone to a human subject by determining the dose of a topical composition comprising the hormone and a penetration enhancer according to the height and weight of the subject and administering the dose of the composition to the skin of the subject. In one embodiment, the hormone is a sex hormone selected from the group consisting of androgen-like compounds, estrogen-like compounds, and progestin like compounds.

In another embodiment, the invention provides a method of restoring serum levels of sex hormone in a human subject to normal levels by i) determining the height and weight of the subject and ii) using the height and weight of the subject to estimate a first dose amount of a topical composition comprising the sex hormone and a penetration enhancer; iii) applying to the skin of the subject the composition in the first dose amount; iv) measuring the level of the sex hormone in the blood of the subject; and v) if the blood sex hormone level is below a first predetermined level treating the subject with a second dose 25 to 100% greater than the first dose amount; or if the blood sex hormone level is above or near a second predetermined level, treating the subject with a third dose which is 25 to 75% less than the first dose amount. In a further embodiment, the sex hormone is an androgen selected from the group consisting of testosterone, its salts, esters, and derivatives. In a still further embodiment, testosterone is measured about two hours after the initial daily applying of the first dose amount. In another embodiment, the testosterone is measured at the blood testosterone steady state. In another embodiment, the testosterone is measured at least three days after the first applying of the composition.

In another embodiment of the method of restoring serum levels of sex hormone in a human subject to normal levels, the topical composition has a pH value of between about 4 to about 8 and comprises the hormone in a concentration of about 0.1% to about 2% w/w, and b) a penetration-enhancing system consisting essentially of (i) a membrane fluidizer comprising oleic acid; (ii) a C₁-C₄ alcohol; and (iii) a glycol. In a still further embodiment, the sex hormone is an androgen, including, but not limited to, testosterone, or an estrogen. In one such embodiment, where the hormone is testosterone or the pharmacologically acceptable salt, ester or derivative of testosterone, the first predetermined level is about 250-350 ng/dl and the second predetermined level is about 1000-1200 ng/dl. In another such embodiment, where the hormone is testosterone or the pharmacologically acceptable salt, ester or derivative of testosterone, the first predetermined level is about 250-350 ng/dl and the second predetermined level is about 1000-1200 ng/dl.

In one embodiment, the invention provides a metered device for delivering a topical composition comprising testosterone and a penetration enhancer to a subject, wherein said metered device is set to deliver a number of identical amounts of the composition wherein the number is determined according to the height or weight of the subject. In a further embodiment, the amounts are set according to the body mass index (BMI) of the subject and the empirically determined relationship between BMI and C_{avg}. In another further embodiment, the amounts are set according to the body weight of the subject and the empirically determined relationship between body weight and C_{avg}.

In some embodiments of each of the above three aspects, the subject is a male with hypogonadism. Such hypogonadism can be the result of inadequate production of testosterone by the Leydig cells of the testes. The etiology of hypogonadism may be primary or secondary. Primary hypogonadism is associated with dysfunction in the testis. Idiopathic primary testicular failure affects approximately 5% of the male population. Less common causes are Kleinfelter's syndrome, bilateral cryptorchidism, myotonic dystrophy, polyglandular failure, gonadal dysgenesis and vanishing testis syndrome. New treatments are needed for the disease. As such, in certain embodiments, the present invention provides methods for treating hypogonadism by administering a therapeutically effective amount of a topical composition comprising testosterone, its pharmaceutically acceptable salts, esters, and derivatives, testosterone-like compounds, or androgens.

In another aspect the invention provides methods for treating subjects with female or male sexual dysfunctions with topical testosterone gel according to their body weight or BMI. Sexual Dysfunction is a commonly diagnosed medical condition. In some embodiments, such male subjects are administered testosterone replacement therapy if the T/SHBG level is <153 nmol/L or bioactive T is <70 ng/dL. For subjects qualified for testosterone replacement therapy, the starting dose of topical testosterone can be determined based on each subject's BMI level to ensure sufficient and efficacious testosterone therapy beginning from Day 1 of the treatment.

For aspects of this invention involving methods of treating both male and female subjects with sexual dysfunction, the initial dose for treating the subjects will be determined according to BMI (or alternatively the body weight) of the subject; and then selecting the individual's dose according to a predetermined empirical relationship between the body weight or BMI, the applied dosage, and the serum level of the hormone in a reference population at steady state. The sex hormone(s) are administered in a semisolid topical gel formulation having a a pH value of between about 4 to about 8 and comprising 0.1% to about 2% w/w of each human sex hormone, a penetration-enhancer such s oleic oleic acid; and a C₁-C₄ alcohol; and a glycol.

In other embodiments, he present invention provides compositions and methods for titrating the amount of a semisolid topical androgen containing pharmaceutical composition to be administered to a female subject suffering from androgen deficiency related conditions. In one such embodiment, a semisolid topical composition comprising a therapeutically effective amount of an androgen (e.g., testosterone from 0.01% to 5% w/w) and an effective amount of a penetration enhancer is applied to the skin of the female subject in an dosage amount determined according to the weight or BMI of the subject and the empirically determined relationship between weight or BMI, the amount of the composition to be applied, and the resulting measured androgen levels in the treated population. In further embodiments, the androgen (e.g. testosterone) serum level is measured in the subject after several days of administration or after a sufficient time for a steady state to have been reached and if the measured levels are below the desired range the amount to be applied is increased and if the measured levels are above the desired range the amount to be applied is decreased. In preferred embodiments, the penetration-enhancing system of the composition consists essentially of (i) a membrane fluidizer comprising oleic acid; (ii) a C₁-C₄ alcohol; and (iii) a glycol.

In another aspect of the invention, methods of treatment are provided for male and female patients suffering from systemic disorders that suppress testosterone levels, including, but not limited to, hepatic cirrhosis, chronic renal failure, sickle cell anemia, thalassemia, hemochromatosis, AIDs virus, amyloidosis, chronic obstructive pulmonary disease (COPD), rheumatoid arthritis, chronic infection, and inflammatory or debilitating conditions. In one such embodiment, a semisolid topical composition comprising a therapeutically effective amount of an androgen (e.g., testosterone from 0.01% to 5% w/w) and an effective amount of a penetration enhancer is applied to the skin of the subject in an dosage amount determined according to the weight or BMI of the subject and the empirically determined relationship between weight or BMI, the amount of the composition to be applied, and the resulting measured androgen levels in the treated population. In yet another aspect of the invention, methods of treatment are provided for male and female patients receiving drugs that affect testosterone levels. Drugs that are known to decrease testosterone levels include GnRH agonists, and antagonists, estrogens, progestins, glucocorticoids, ketoconazole, aldactone, thiazide diuretics, opiates, anabolic steroids, amiodarone, and a number of psychotropic agents. Agents that impair testosterone action at the receptor level include aldactone, cimetidine, flutamide, and other androgen antagonists. In one such embodiment, a semisolid topical composition comprising a therapeutically effective amount of an androgen (e.g., testosterone from 0.01% to 5% w/w) and an effective amount of a penetration enhancer is applied to the skin of the subject in an dosage amount determined according to the weight or BMI of the subject and the empirically determined relationship between weight or BMI, the amount of the composition to be applied, and the resulting measured androgen levels in the treated population.

In another aspect of the invention, methods of treatment are provided for male and female patients suffering from spacial cognition difficencies. In one such embodiment, a semisolid topical composition comprising a therapeutically effective amount of an androgen (e.g., testosterone from 0.01% to 5% w/w) and an effective amount of a penetration enhancer is applied to the skin of the subject in an dosage amount determined according to the weight or BMI of the subject and the empirically determined relationship between weight or BMI, the amount of the composition to be applied, and the resulting measured androgen levels in the treated population.

In one of its aspects the invention provides methods for treating chronic fatigue syndrome by hormone replacement therapy. In some of its embodiments, the invention provides methods of treating patients suffering from chronic fatigue syndrome with 0.5 mg, 1 mg, or 1.5 mg testosterone in the form of a transdermal testosterone gel containing a penetration enhancer. The dosage of testosterone given is based on the patient's BMI or body weight. In another embodiment, sex hormone(s) are administered in a semisolid topical gel formulation having a a pH value of between about 4 to about 8 and comprising 0.1% to about 2% w/w of each human sex hormone, a penetration-enhancer such s oleic oleic acid; and a C₁-C₄ alcohol; and a glycol.

In some embodiments, the present invention provides methods for treating chronic Epstein-Barr virus infection with a semisolid topical Testosterone gel in which patients suffering from chronic Epstein-Barr virus infections are treated with 1 mg, 2 mg, or 3 mg testosterone in the form of a transdermal testosterone gel containing a penetration enhancer. The initial dose for treating the subjects will be determinined according to the body weight and/or BMI of the subject; and then selecting the individual's dose according to a predetermined empirical relationship between the body weight or BMI, the applied dosage, and the serum level of the hormone in a reference population at steady state. The sex hormone(s) are administered in a semisolid topical gel formulation having a a pH value of between about 4 to about 8 and comprising 0.1% to about 2% w/w of each human sex hormone, a penetration-enhancer such s oleic oleic acid; and a C₁-C₄ alcohol; and a glycol.

In some embodiments according to the present invention young girls needing pubertal development or adults with Turner's syndrome are treated with a semisolid topical composition comprising sex hormones. The hormones may be estrogen-like, progestin-like, androgen-like or a mixture thereof.an estrogen. For instance, girls and women with Turner's Syndrome between the ages of 14 and 50 years can be administered hormone replacement therapy by semisolid topical gels.

In some embodiments in each of the three aspects, the semisolid topical composition, is formulated as a solution, cream, lotion, ointment, or gel.

In another embodiment, the present invention provides a method for maintaining the ratio of 5-αdihydrotestosterone level (DHT) to testosterone level (DHT:T) at approximately 1:10, comprising: administering a therapeutically effective amount of a topical composition comprising testosterone, thereby maintaining the ratio of 5-αdihydrotestosterone level (DHT) to testosterone level (DHT:T) at approximately 1:10.

In yet another embodiment, the present invention provides a method of restoring serum levels of testosterone to about 300 to 1140 ng/dL, comprising: administering a therapeutically effective amount of a topical composition comprising testosterone and an effective amount of a penetration enhancer, thereby restoring serum levels of testosterone to about 300 to 1140 ng/dL.

In other aspects, the present invention provides a semisolid testosterone gel for maintaining the serum testosterone level in the physiological range of hypogonadal males.

In another embodiment, the subject is a female with an androgen deficiency. In some such embodiments the subject is a woman with increasing age, or a women who after either natural or surgical menopause has impaired sexual function, lessened well-being, loss of energy, and negative effects on bone and muscle mass. In one embodiment, the invention provides methods for increasing bioactive androgen (e.g. testosterone) levels. In some embodiment, the present invention provides a method for maintaining serum levels of bioactive testosterone in a menopausal mammalian female by administering a semisolid topical composition comprising a therapeutically effective amount of a testosterone and an effective amount of a skin penetration enhancer, thereby maintaining serum levels of bioactive testosterone in the menopausal mammalian female. In certain embodiments, the topical composition of testosterone has concentration of about 0.01% to about 5% w/w. Preferably, the concentration is about 0.25% to about 0.5% w/w. The topical composition of testosterone may further comprises ethanol and a carbomer. The topical composition is formulated in solutions, creams, lotions, ointments, aerosols and gels. The serum levels of bioactive testosterone which are preferably achieved after administration of the testosterone to a female with androgen deficiency is about 1.1 to about 14.4 ng/dL.

In certain aspects, the methods of the present invention provide the optimum dose and tolerability of testosterone gel that best provides serum levels of bioactive testosterone in surgically induced menopausal women that approximate the upper one third range in young women. In one such embodiment, the invention provides a method for restoring libido in a menopausal mammalian female, comprising administering a therapeutically effective amount of a topical composition comprising testosterone, thereby restoring libido in the menopausal mammalian female. In yet another aspect, the present invention provides a method for increasing bone density in a menopausal mammalian female, comprising administering a therapeutically effective amount of a topical composition comprising testosterone, thereby increasing bone density in the menopausal mammalian female. In such aspects, the invention provides semisolid topical compositions comprising a therapeutically effective amount of testosterone or another androgen and an effective amount of a skin penetration enhancer and a method for determining the amount of the composition to apply to such female subjects. In one embodiment, the method involves the steps of determining the weight or BMI of the female subject and adjusting the dose of testosterone to be applied according to the empirically derived relationship between body mass or BMI, the dose applied, and the resulting measured serum testostereone level in a representative population.

### BRIEF DESCRIPTION OF THE DRAWINGS

FIG. 1 illustrates the mean (±SD) testosterone concentrations at baseline (Pretreatment) for the subset of subjects with baseline data (N=10).

FIG. 2 illustrates the mean total serum testosterone concentration-time profile for all day for 42/56 MITT subjects on day 1 (N=163).

FIG. 3 illustrates mean total serum testosterone concentration-time profile on day 14 for the day 42/56 MITT subjects (N=163).

FIG. 4 illustrates mean total serum testosterone concentrations on day 14 by final dose group for the day 42/56 MITT Population (N=163).

FIG. 5A illustrates mean total serum testosterone concentrations on day 14 by final dose groups for the day 42/56 MITT subjects who completed the study through day 182 (N=146).

FIG. 5B illustrates mean total serum testosterone concentrations on day 42/56 by final dose groups for the day 42/56 MITT subjects who completed the study through day 182 (N=146).

FIG. 5C illustrates mean total serum testosterone concentrations on day 182 by final dose groups for the day 42/56 MITT subjects who completed the study through day 182 (N=146).

FIG. 6 illustrates mean Cₘᵢₙ, C_{avg}, Cₘₐₓ at days 1, 14, 42/56, and 182 in the 3 g final dose group for the day 42/56 MITT population (N=71).

FIG. 7 illustrates man C_{avg} and Cₘₐₓ before (day 14) and after (days 42/56 and 182) dose adjustment in the three dose subgroups (N=163) of the MITT population.

FIG. 8 illustrates the correlation between day 14 C_{avg} and BMI for day 42/56 for the MITT population.

FIG. 9 illustrates the mean (SD) testosterone concentrations at baseline (Pretreatment) and at day 42/56 for the subset of subjects with baseline data (N=10).

FIG. 10 illustrates the cumulative proportion of subjects by duration (hrs.) of concentration <300 ng/dL for subjects with C_{avg} between 300 and 1140 ng/dL on PK for day 42/56 in the Efficacy Evaluable subjects with C_{avg} within the PR.

FIG. 11 illustrates the increase in C_{avg} during CP601B treatment in MITT subjects with no measurable pretreatment testosterone (N=15).

FIG. 12 illustrates the mean Cavg and Cmax before dose adjustment (Day 14) when all subjects were using 3 g CP601B, by final dose group (n=163)for day 42/56 MITT population.

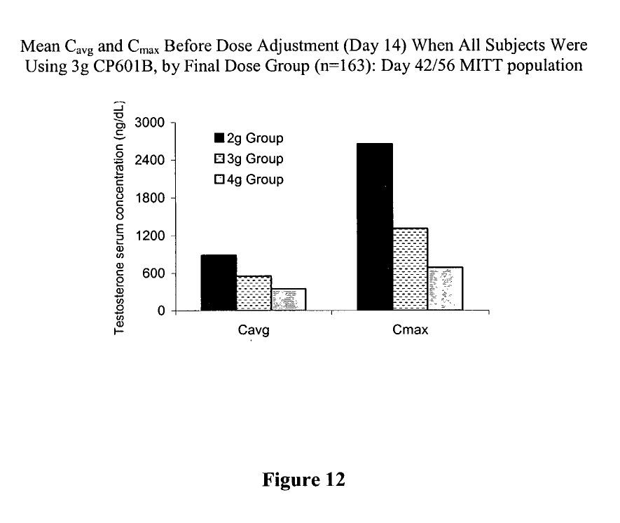

FIG. 13 illustrates the mean total serum testosterone concentrations on day 14 by final dose group for day 42/56 MITT population (N=163).

FIG. 14 illustrates the linear regression of key day 14 PK parameters (Cmax, Cavg, Cmin) for day 14 C₂ for the ITT population.

FIG. 15 illustrates a preliminary dose response determination of CP601 in males with hypogonadism.

FIG. 16 illustrates the correlation between BMI and C_{avg} value of the total testosterone concentration (ng/dl) in female subjects.

### DETAILED DESCRIPTION OF THE INVENTION AND PREFERRED EMBODIMENTS

Two measures, C_{avg} and Cₘᵢₙ of a serum mammalian hormone concentration time course following administration of a hormonal therapy are particularly useful parameters for predicting therapeutic efficacy. Cₘᵢₙ is the minimal serum or plasma concentration of the hormone over some predetermined time period or interdosing interval. C_{avg} is the average serum or plasma concentration of the hormone over some predetermined time period or interdosing interval. Cₘₐₓ is useful as a predictor of the potential for overtreatment. Generally, pharmacodynamic and therapeutic effects are better correlated with such measures than with the dosage or the amount of the composition applied to a subject. The reason lies in the intersubject variability in rates of metabolism and bioavailability from the topical route of administration. Thus, to maximize a successful outcome if the drug is effective and to minimize adverse drug reactions, clinical trial designs and therapy with mammalian hormones are based on drug blood levels rather than dosage.

For instance, clinical trials of topical preparations of mammalian hormones frequently follow the paradigm of administering a predetermined dose of the pharmaceutical composition to the experimental subjects, measuring the serum level of the hormone at steady state, and then adjusting the individual dosage according to the measured serum level (e.g., increasing the dose if the serum level of the hormone is below the desired range, decreasing the dose if the serum level is above the desired range). However, if a mammalian hormone does not sufficiently penetrate the skin, increases in the topically applied amount of the hormonal composition for an individual may not be reflected as increases in the subsequent serum hormone level measurements for the individual (see WO 02/17926).

The clinical methodologies which focus on blood levels for predicting and monitoring efficacy do not therefore rely upon body weight or BMI of a subject in calculating the amount of the mammalian hormone to be applied in a semisolid topical composition. The high individual variability observed for skin penetration in studies using semisolid topical compositions to administer mammalian hormones teaches away from relying upon such a simple and traditional parameter as body weight. Surprisingly, in the course of doing the traditional pharmacokinetic and pharmacodynamic studies of a mammalian hormone administered via a semisolid topical composition having an effective amount of a penetration enhancer, it was found that a subject's body weight or even more particularly relative amount of body fat (e.g., using BMI as a surrogate measure) was strongly associated with serum levels of the hormone at steady state and that dosing methodologies could be improved by incorporating such information as body weight and body height (e.g., BMI) into, at least, the initial dose determination which dosage might then be fine-tuned based upon measured serum hormone levels as steady state.

An advantage of such a method of determining doses is a greater likelihood of adequately dosing and not overdosing or underdosing a subject with hormone throughout the sometimes lengthy initial period it takes to achieve steady state. This provides benefit to the medical practitioner prescribing the active as this method of determining doses does not require an initial blood draw to determine initial dosing levels. Additionally, the method provides benefits to both the patient and the practitioner, as it greatly increases the likelihood the patient will be treated with the proper dosage from the initial dose and thus will accrue the benefits of the treatment more rapidly.

Definitions

It is noted here that as used in this specification and the appended claims, the singular forms “a,” “an,” and “the” include plural reference unless the context clearly dictates otherwise.

Unless defined otherwise, all technical and scientific terms used herein have the same meaning as commonly understood by those of ordinary skill in the art to which this invention belongs. The following references provide one of skill with a general definition of many of the terms used in this invention: Singleton, et al., Dictionary Of Microbiology And Molecular Biology (2d ed. 1994); The Cambridge Dictionary Of Science And Technology (Walker ed., 1988); and Hale & Markham, The Harper Collins Dictionary Of Biology (1991).

The terms “treatment,” “therapy” and the like include, but are not limited to, changes in the recipient's status. The changes can be either subjective or objective and can relate to features including, but not limited to, symptoms or signs of the disease or condition being treated. Preventing the deterioration of a recipient's status is also included by the term. Therapeutic benefit includes any of a number of subjective or objective factors indicating a response of the condition being treated.

“Drug”, “pharmacological agent”, “pharmaceutical agent”, “active agent”, and “agent” are used interchangeably and are intended to have their broadest interpretation as to any therapeutically active substance, including mammalian hormones, which is delivered to a living organism to produce a desired, usually beneficial effect.

A “semisolid topical composition” refers to a composition in the form of a lotion, a cream, a gel, or an ointment and which is formulated for direct application to the skin or mucous membranes. Such compositions may contain a variety of compounds and ingredients.

Body Mass Index (BMI) is the body weight expressed in kilograms divided by the square of the body height expressed in meters. Thus, BMI values, whether expressed or not, are normally in units of kg/m². In the English system of units, this BMI can be derived by the formula: Weight in pounds÷(Height in inches)²×703. Methods of determining body weight and body mass index are well known in the art.

“Pharmaceutically-acceptable” or “therapeutically-acceptable” refers to a substance which does not interfere with the effectiveness or the biological activity of the active ingredients and which is not toxic to the hosts, which may be either humans or animals, to which it is administered.

“Therapeutically-effective amount” refers to the amount of an active agent sufficient to induce a desired biological result in the instant formulation. That result may be alleviation of the signs, symptoms, or causes of a disease, or any other desired alteration of a biological system. The term “therapeutically effective amount” is used herein to denote any amount of the compound or formulation that causes a substantial improvement in a disease condition when applied to the affected areas repeatedly over a period of time. The amount will vary with the hormone, the condition being treated, the stage of advancement of the condition, and the type and concentration of formulation applied, and particularly the subject. To exemplify, in some embodiments, a therapeutically effective amount may range from 0.1 mg to 1 g of a mammalian hormone being applied to the skin in a formulation dose of from about 0.1 to 10 grams or 2 to 20 grams. In some embodiments, the amount of the hormone applied to the skin is 25 to 500 mg. To achieve such or other applied amounts of hormone, the concentration of the hormone in the formulation can be varied according to the desired amount and the amount of the formulation to be applied.

The term “pharmaceutically acceptable carrier” encompasses any of the standard pharmaceutical carriers, buffers and excipients, including phosphate-buffered saline solution, water, and emulsions (including, but not limited to, an oil/water or water/oil emulsion), and various types of wetting agents and/or adjuvants. Suitable pharmaceutical carriers and their formulations are described in REMINGTON'S PHARMACEUTICAL SCIENCES (Mack Publishing Co., Easton, 19th ed. 1995). Preferred pharmaceutical carriers depend upon the intended mode of administration of the active agent. Typical modes of administration are described below.

The term “effective amount” means a quantity sufficient to produce a desired result. The desired result may comprise a subjective or objective improvement in the recipient of the dosage. With respect to a penetration enhancer, the effective amount substantially increases penetration of the hormone or active through the skin over a given period of time upon application of the composition dose as compared to the penetration of a hormone or active from a formulation lacking the enhancer. A substantial increase in penetration is at least 100%, and more preferably is at least 200% or at least 300%. In some embodiments, the compositions may be applied in amounts of from about 0.1 to 10 g. More preferably pharmaceutical compositions are applied in an amount of from about 0.5 to 5 g, and still more preferably in amounts from about 1 to 4 g.

The term “effective concentration” means an amount sufficient to produce the desired result upon application of the dose of the composition. The desired result may comprise a subjective or objective improvement in the recipient of the dosage. With respect to a penetration enhancer, the effective amount substantially increases penetration of the hormone through the skin over a given period of time upon application of the composition dose. A substantial increase is at least 100%, and more preferably is at least 200% or at least 300%. In some embodiments, the compositions may be applied in amounts of from about 0.1 to 10 g. More preferably pharmaceutical compositions are applied in an amount of from about 0.5 to 5 g, and still more preferably in amounts from about 1 to 4 g. An effective concentration of a penetration enhancer, depends upon the enhancer's abilty to prmote skin penetration. In some embodiments, for instance, the enhancer may be in a w/w concentration ranging from about 0.1% to 5% or from 1% to 10%; or from 10% to 50% or more.

A “prophylactic treatment” is a treatment administered to a subject who does not exhibit signs of a disease or exhibits only early signs of a disease, wherein treatment is administered for the purpose of decreasing the risk of developing pathology.

A “therapeutic treatment” is a treatment administered to a subject who exhibits signs of pathology, wherein treatment is administered for the purpose of diminishing or eliminating those pathological signs.

Mammalian Hormones

The hormones of the invention include compounds secreted or released by various cells in the body of mammals which are then carried in the blood stream to reach the target cell, tissue or organ upon which their effects are produced. In one embodiment, the hormones are human hormones. Suitable hormones for use with the methods of the present invention are well known in the art and are described, e.g., in Goodman and Gilman, The Pharmacological Basis of Therapeutics (9th Ed.), McGraw-Hill, Inc. (1996); The Merck Index (12th Ed.), Merck & Co., Inc. (1996); The Physician's Desk Reference (49th Ed.), Medical Economics (1995); and Drug Facts and Comparisons (1993 Ed.), Facts and Comparisons (1993).

Examples of such hormones include, but are not limited to, somatotrophic hormones (e.g., growth hormone, prolactin (Prl), placental lactogen (PL)); glycoprotein hormones (e.g., luteinizing hormone (lutropin, LH), follicle-stimulating hormone (follitropin, FSH), chorionic gonadotropin (CG, choriogonadotropin, pregnyl, A.P.L., profasi); menotropins (e.g., pergonal, human menopausal gonadatropins (hMG), urofollitropin (uFSH), metrodin); thyroid-stimulating hormone (TSH, thyrotropin)); POMC-derived hormones (e.g., corticotropin (ACTH), α-melanocyte-stimulating hormone (α-MSH), β-melanocyte-stimulating hormone (β-MSH), β-lipotropin (β-LPH), γ-lipotropin (γ-LPH)); somatostain (e.g., somatropin, recombinant, humatrope, somarem, protropin); thyroid hormones (e.g., thyroxin (levothyroxine sodium), triiodothyronine (liothyronine sodium), 3,5,5′-triiodothyronine); thyroid-releasing hormone (TRH); growth hormone-releasing hormone (GHRH), somatostatin (e.g., somatostatin-28, somatostatin-14, octrotide, sandostatin), gonadotrophin-releasing hormone (GnRH) (e.g., decapeptyl, leuprolide, buserelin, nafarelin, deslorelin, histrelin, ganirelix, gonadorelin hydrochloride (e.g., factrel, letrepulse), leuprolide acetate (lupron), histrelin acetate (synarel), goserelin acetate (zoladex)); dopamine; thyrotropin, thyrotropin-releasing hormone; adrenocorticotropin (ACTH, corticotropin, cosyntropin); corticotropin-releasing hormone; parathyroid hormone (PTH) and the pharmaceutically acceptable salts, esters, derivatives, metabolites, mimetics, or synthetic analogs thereof (See, e.g., Goodman and Gilman, supra).

Preferred hormones according to the invention are sex hormones (e.g., androgens, estrogens, and progestins) as well as corticosteroids and glucocorticoids. The term hormones includes compounds which interact with the hormone receptor including, but not limited to, hormone receptor agonists or antagonists.

The hormone can be a testosterone-like compound, estrogen-like compound, progestin-like compound, adrenocorticoid, glucocorticoid or mineralcorticoid and the pharmaceutically acceptable biologically active salts, esters, derivatives, metabolites, mimetics, or synthetic analogs and mixtures thereof. (see, e.g., Goodman and Gilman, supra) Testosterone-like compounds include, but are not limited to, testosterone, testosterone propionate, testosterone enanthate, testosterone cypionate, testosterone undecenoate, dihydrotestosterone, danazol, fluoxymesterone, methyltestosterone, oxandrolone, DHEA and tibolone and the pharmaceutically acceptable salts, esters derivatives, metabolites, mimetics, or synthetic analogs and mixtures thereof; (see, e.g., Goodman and Gilman, supra).

The hormone can be an estrogen like compound, including but not not limited to, 17-β-estradiol, estrone, mestranol, estradiol valerate, estradiol dypionate, ethynyl estrodil, quinestrol, estrone sulfate, phytoestrogens including, but not limited to, flavones, isoflavones (e.g., genistein), resveratrol, coumestan derivatives, and the pharmaceutically acceptable salts, esters, derivatives, metabolites, mimetics, or synthetic analogs and mixtures thereof. Estrogen-like compounds include those compounds that bind to the estrogen receptor and act as agonists thereof. (see, e.g., Goodman and Gilman, supra).

The hormone can be a progestin-like compounds, (e.g., progesterone, hydroxyprogesterone caproate, medroxyprogesterone acetate, 19-nortestosterone, norethynodrel, norgestrel, desogestrel, norgestimate, norethindrone (norlutin), norethindrone acetate (norlutate, aygestin)) and the pharmaceutically acceptable salts, esters, derivatives, metabolites, mimetics, or synthetic analogs and mixtures thereof. (see, e.g., Goodman and Gilman, supra).

The physicochemical properties (e.g., molecular weight, hydrophobicity, hydrophilicty) of an agent help to determine its skin penetration. In preferred embodiments, the composition to be applied to skin has a pH between 4 and 8 and comprises a mammalian hormone with a similar physicochemical profile (molecular weight, water solubility) as testosterone and an effective amount of a penetration enhancer including, but not limited to, oleic acid; a carbomer, an alcohol, and a gelling agent. Such hormones include, but are not limited to, hormones derived from cholesterol or having a sterol structure. Such hormones include androgens, estrogens, and progestins as well as adrenocorticoids, mineralocorticoids, and glucocorticoids.

Penetration Enhancers

The rate of systemic delivery of topically active agents (e.g., mammalian hormones) is largely controlled by the rate of their permeation through skin. A penetration enhancer is an agent known to increase or accelerate the delivery of active agents through the skin. Such agents can be used to modulate the penetration of an agent through skin and can be selected according to the hormone or amount of enhancement desired. Penetration enhancers are also referred to as accelerants, adjuvants, and sorption promoters. Examples of penetration enhancers suitable for use according to the invention include C₈-C₂₂ fatty acids (e.g., isostearic acid, octanoic acid, oleic acid); C₈-C₂₂ fatty alcohols (e.g., oleyl alcohol, lauryl alcohol); lower alkyl esters of C₈-C₂₂ fatty acids (e.g., ethyl oleate, isopropyl myristate, butyl stearate, methyl laurate); di-lower alkyl esters of C₆-C₈ diacids (e.g., diisopropyl adipate); monoglycerides of C₈-C₂₂ fatty acids (e.g., glyceryl monolaurate); tetrahydrofurfuryl alcohol polyethylene glycol ether; polyethylene glycol; propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

Additional examples of compounds known to act as penetration enhancers can be found in the literature. See, e.g., Lenneruas H et al., J Pharm Pharmacol 2002 April;54(4):499-508; Karande P and Mitragotri S, Pharm Res 2002 May;19(5):655-60; Vaddi H K et al., J Pharm Sci 2002 July;91(7):1639-51; Ventura C A et al., J Drug Target 2001;9(5):379-93; Shokri J et al., Int J Pharm Oct. 9, 2001;228(1-2):99-107; Suzuki A et al., Biol Pharm Bull 2001 June;24(6):698-700; Alberti I et al., J Control Release Apr. 28, 2001;71(3):319-27; Goldstein I et al., Urology 2001 February;57(2):301-5; Kiijavainen M et al., Eur J Pharm Sci 2000 April;10(2):97-102; and Tenjarla S N et al., Int J Pharm Dec. 10, 1999;192(2):147-58; all of which are incorporated by reference in their entirety.

Oleic acid is a preferred penetration enhancer. Other related suitable penetration enhancers can be used. Such enhancers include the fatty acid homologues and derivatives of oleic acid (e.g., oleyl alcohol and esters of oleic acid).

Compounds known to intercolate in lipid bilayers (e.g., laurocapram (Azone™)) and saturated and unsaturated long-chain fatty acids are also suitable for used as penetration enhancers.

In vitro methods suitable for testing and selecting penetration enhancers, and for determining optimal concentrations for enhancing the penetration of a given hormone, are known to one of ordinary skill in the art.

The penetration enhancers are present in an amount effective for enhancing the penetration of the mammalian hormone across the skin and into the blood or serum. In vitro and in vivo methods may be used for determining the effective concentration of a penetration enhancer.

The in vitro evaluation of compositions of the present invention can be accomplished using a variety of skin diffusion cell experimental protocols. (See, for example, “Transdermal Drug Delivery” Ed. Jonathan Hadgraft et al., Chapter 9, Marcel Dekker Inc., New York; Bronaugh et al., J. Phar. Sci., 75:1094-1097, (1986); and Bronaugh et al., J. Phar. Sci., 74: 64-67, (1985)). In general in vitro transdermal delivery experiments are conducted on either vertically or horizontally arranged diffusion cells. It is desirable to control various environmental factors that can effect the rate of diffusion. The factors include, for instance, temperature. This is because the rate of diffusion will increase with increasing temperature. Thus, it is important to consider various factors related to the skin surface including, skin surface coverings, microorganisms, vehicle formulation and duration of contact with the skin.

In vitro methods for assessing the penetration enhancement of enhancers are also taught in U.S. Pat. No. 6,319,913, which is herein incorporated by reference. In vivo methods of assessing the degree of enhancement afforded by a particular amount or concentration of a penetration enhancer involve the measurement of the serum levels of the hormone following the topical application of the composition and comparing those levels to those provided by a similar composition lacking a penetration enhancer. Such methods of monitoring serum hormone levels are well known in the art. Preferably, range finding is conducted in vitro before conducting the in vivo studies.

Pharmaceutical Formulations

The pharmaceutical compositions of the invention comprise a therapeutic amount of a mammalian hormone and an effective amount of a skin penetration enhancer. Suitable compositions for use according to the invention are taught in U.S. Pat. No. 6,319,913B 1 which is incorporated herein by reference in its entirety. U.S. Pat. No. 6,319,913B1 further discloses compositions of an active agent with skin penetration enhancers and preferably, oleic acid, as a skin penetration enhancer.

In some embodiments, the compositions of the present invention include a mammalian hormone in a concentration of about 0.1% to about 2% w/w, and a penetration-enhancing system consisting essentially of (i) a membrane fluidizer comprising oleic acid in an amount ; (ii) a C₁-C₄ alcohol; and (iii) a glycol said composition having a pH value of between about 4 to about 8. Such compositions provide a systemically active mammalian hormone composition with increased penetration or systemic delivery of the hormone. Optionally, a gelling agent may also be included.

In addition to the mammalian hormone and oleic acid, or another penetration enhancer, the pharmaceutical compositions of the present invention may contain an alcohol. As used herein the term “alcohol” refers to a monohydric alcohol, preferably an aliphatic alcohol and more preferably a saturated monohydric aliphatic alcohol. Examples are methanol, ethanol, propanol, isopropanol, and octanol. In the present invention, a C₁-C₄ alcohol is suitable. These include, but are not limited to, ethanol, propanol, isopropanol and mixtures thereof. Mixtures include, for example, ethanol and isopropanol.

Such compositions in accordance with the present invention may contain an alcohol in about 5% to about 55% weight to weight of the composition. Preferably, the alcohol is present from about 10% to about 40% weight to weight and more preferably, from about 25% to about 35% weight to weight of the composition.

In addition to the mammalian hormone, penetration enhancer (e.g.oleic acid) and an alcohol, the compositions of the present invention may comprise a glycol. As used herein, the term “glycol” refers to a polyhydric alcohol, preferably a dihydric alcohol. Examples are ethylene glycol, propylene glycol, butylene glycol and glycerol. In some embodiments, the glycol is ethylene glycol, propylene glycol, butylene glycol and mixtures thereof.

In some embodiments, the compositions of the present invention contain a glycol in about 25% to about 55% weight to weight of the composition. In some embodiments, the glycol content is from about 30% to about 40% weight to weight of the composition.

In some embodiments, gelling agents are included in the pharmaceutical compositions. Suitable gelling agents of the present invention include, but are not limited to, carbomers, including Carbopol 1342, Carbopol 1382, and Carbopol 940; Klucel and Klucel HF. Synonyms for carbomer include carbopol, poly(1-carboxyethylene) or poly(acrylic acid). Those of skill in the art will know of other gelling agents that are suitable to practice the present invention. The gelling agent can be present from about 1% to about 10% weight to weight of the composition. Preferably, the gelling agent is present from about 1% to about 5% w/w, and more preferably, from about 1% to about 3% weight to weight of the composition.

Dosage forms for the semisolid topical administration of the mammalian hormones of this invention include ointments, pastes, creams, lotions, and gels. The dosage forms may be formulated with mucoadhesive polymers for sustained release of active ingredients at the area of application to the skin. The active compound may be mixed under sterile conditions with a pharmaceutically acceptable carrier, and with any preservatives, buffers, or propellants, which may be required. Such topical preparations can be prepared by combining the compound of interest with conventional pharmaceutical diluents and carriers commonly used in topical liquid, cream, and gel formulations.

Ointment and creams may, for example, be formulated with an aqueous or oily base with the addition of suitable thickening and/or gelling agents. Such bases may include water and/or an oil including, but not limited to, liquid paraffin or a vegetable oil including, but not limited to, peanut oil or castor oil. Thickening agents which may be used according to the nature of the base include soft paraffin, aluminum stearate, cetostearyl alcohol, propylene glycol, polyethylene glycols, woolfat, hydrogenated lanolin, beeswax, and the like.

Lotions may be formulated with an aqueous or oily base and, in general, also include one or more of the following: stabilizing agents, emulsifying agents, dispersing agents, suspending agents, thickening agents, coloring agents, perfumes, and the like. The ointments, pastes, creams and gels also may contain excipients, including, but not limited to, animal and vegetable fats, oils, waxes, paraffins, starch, tragacanth, cellulose derivatives, polyethylene glycols, silicones, bentonites, silicic acid, talc and zinc oxide, or mixtures thereof.

Suitable excipients, depending on the hormone, include petrolatum, lanolin, methylcellulose, sodium carboxymethylcellulose, hydroxpropylcellulose, sodium alginate, carbomers, glycerin, glycols, oils, glycerol, benzoates, parabens and surfactants. It will be apparent to those of skill in the art that the solubility of a particular compound will, in part, determine how the compound is formulated. An aqueous gel formulation is suitable for water soluble compounds. Where a compound is insoluble in water at the concentrations required for activity, a cream or ointment preparation will typically be preferable. In this case, oil phase, aqueous/organic phase and surfactant may be required to prepare the formulations. Thus, based on the solubility and excipient-active interaction information, the dosage forms can be designed and excipients can be chosen to formulate the prototype preparations.

The topical pharmaceutical compositions can also include one or more preservatives or bacteriostatic agents, e.g., methyl hydroxybenzoate, propyl hydroxybenzoate, chlorocresol, benzalkonium chlorides, and the like. The topical pharmaceutical compositions also can contain other active ingredients including, but not limited to, antimicrobial agents, particularly antibiotics, anesthetics, analgesics, and antipruritic agents.

One example of a topical formulation includes, in addition to the 1% w/w of the mammalian hormone, 75% (w/w) white petrolatum USP, 4% (w/w) paraffin wax USP/NF, lanolin 14% (w/w), 2% sorbitan sesquioleate NF, 4% propylene glycol USP, and 1% compound of the present invention.

In one embodiment, the mammalian hormone is formulated as composition having a pH from about 4 to 8 and comprising about 15% ethanol, about 15% isopropanol, about 10-50% propylene glycol, about 0.2 to 5% oleic aid, about 0.1 to 5% hormone, about 0.2% to 5% carbomer, about 0.1 to 1% triethanolamine; and water to make up 100% (percents as w/w). In some embodiments, the pH is between 6.5 and 7.5. The mammalian hormone would include a sex hormone, including androgens, estrogen-like agents, and progestins; a mineralocorticoid, adrenocorticoid, or glucocorticoids.

Therapeutic Kits

In one of its embodiments, the invention provides a kit comprising a container holding a semisolid topical composition having a therapeutically effective amount or concentration of a mammalian hormone and an effective concentration or amount of a penetration enhancer. A preferred enhancer is oleic acid. A preferred hormone is testosterone, dihydrotestosterone, estrogen, or estradiol. The container is combined with a pump which dispenses the composition when activated. Pumps for dispensing semisolids are well known in the art. A preferred kit has the pump and container as an integrated members. In some embodiments, when actuated the pump delivers a preset amount ranging from 0.1 to 10 g from an exemplary integral pump of a tapered well metered dose design is disclosed in U.S. patent application Ser. No. 10/197627 filed on Jul. 15, 2002 which is assigned to the same assignee as the present application and is herein incorporated by reference in its entirety. The kit optionally comes with instructions as to how to operate the pump, or how or where to apply the dispensate to the skin, or the need to or how to avoid contamination or exposure of other persons to the treated skin surface, and/or packaging to protect the integrity of the kit components.

Procedure for the Preparation of a Typical Batch of a Pharmaceutical Composition

Starting materials and methods for preparing pharmaceutical compositions of the present invention are well known in the art. One exemplary method of preparing such a pharmaceutical composition is provided herein:

1) Charge 95% of purified water, USP to a suitable vessel and add Carbomer, USP with mixing. Mix the slurry at 20-30° C. until the Carbomer is completely dispersed and hydrated.

2) Add dehydrated ethanol, USP, 2-propanol, USP, oleic acid, butylated hydroxytoluene, NF, and propylene glycol, USP and the mammalian hormone to the primary compounding vessel. After each addition, the mixture is stirred to complete dissolution.

3) Add the entire amount of Carbomer gel to the compounding vessel with good stirring. Add triethanolamine, NF slowly with mixing to thicken the gel.

4) Check the pH and adjust the pH to target using extra triethanolamine, NF or 1.0 N HCI, USP, if necessary. Then q.s. to the final weight with purified water, USP.

5) Samples are taken from top, middle and bottom of the compounding vessel for in-process assays for ethanol and pH.

6) Fill the bulk gel into containers/bottles.

Methods of Treatment

In one of its aspects, the invention provides a method of determining the initial dose of a semisolid topical pharmaceutical composition to administer to a subject. In some embodiments, the subject is a person who is deficient in the hormone or resistant to its effect or otherwise deficient in the biological activity or effects enhanced by administration of the hormone. Such hormonally related conditions or diseases are well known to one of ordinary skill in the art. In some embodiments, the hormone is a sex hormone, a steroid hormone, an estrogen, a progestin, an androgen, a mineralocorticoid or glucocorticoid. Deficiency conditions for such hormones are particularly well known to one of ordinary skill in the art.

In this aspect, the composition is administered to each of a population of human subjects who are of sufficiently diverse weight or BMI to model the effects of body weight or body mass index or subcutaneous fat on the serum levels of the hormone resulting from the administration of the composition in one or more amounts. The serum measures are preferably made at steady state where the therapeutically targeted level is the steady state. In some embodiments, the measures are Cₘₐₓ, Cₘᵢₙ, C_{avg}. or C_{fi} where “fi” stands for “fixed interval.” In one embodiment, the fixed interval is about 2 hours or from 2-4 hours after administration of the hormone.

The relationship between the body weights, BMI's, or subcutaneous fat thickness measures of the subjects, the amount of the composition administered, and the resulting serum hormone levels may thereupon assessed graphically and/or by mathematical analysis so as to describe the relationship between body mass, BMI, or subcutaneous fat thickness measures, the administered dose (if more than one), and the serum hormone levels observed. Methods for assessing body weight, body mass index, and subcutaneous fat are well known to one of ordinary skill in the art.

Subsequently, upon the determination of the body weight, BMI, or subcutaneous fat thickness of the patient/subject, the amount of an initial dosage amount of the composition to be topically applied to the patient can be determined and applied to the patient. After a period of such dosings, the steady state serum level of the hormone can be determined in the subject and the dose adjusted upward or downward according to whether particular desired serum hormone levels have been reached or exceeded.

Mammalian hormones have long been the subject of clinical research, diagnosis and therapy. Methods for measuring the serum levels of such hormones, particularly human hormones, are well known to one of ordinary skill in the art.

Depending on the formulation and the subject, particularly the concentration of the active agent and the amount of the penetration of enhancer, in some embodiments from about 0.5 to about 10 grams (e.g., about 1, 2, 3, 4, 5, 6, 7, 8, 9, or 10 grams) of the composition may be applied directly to skin. More preferred amounts to be applied range from about 1.0 to 5 grams, including about 1, 2, 3, or 4 grams.

Preferred locations for applying the composition include the upper arms, portions of the thighs, and upper back. One or areas may be treated on the same subject. The total surface area, in part, depends upon the amount of the composition to be applied. In some embodiments, 0.1 to 10 g are to be applied. In some embodiments, the treated skin surface area is from about 10 cm² 1,000 cm². The rate of application of the composition, in some embodiments, is about from 0.1 g to 1 g per 100 cm² of skin. In other embodiments, the rate of application is about 1 g to 5 g per 100 cm².

### EXAMPLES

The following examples are offered to illustrate, but not to limit the claimed invention.

### Example 1

Testosterone-deficiency Therapy in Hypogonadal Males Using CP601B

In a further aspect, the invention is drawn to methods and compositions for maintaining serum sex hormone levels in a subject at a desired or predetermined level. The issue of what constitutes an optimum replacement dose or serum level of a sex hormone, however, is complex.

Pharmacodynamic (PD) and therapeutic effects are better correlated with blood levels than with dosage. The reason lies in the intersubject variability in rates of metabolism, in bioavailability from any route of administration, particularly skin absorption, and in other factors. Thus, to maximize a successful outcome if the drug is effective and to minimize adverse drug reactions, in preferred embodiments individual drug blood levels are used to guide dosage adjustments.

In some embodiments, the sex horomone replacement therapy is to achieve and maintain the average serum hormone concentrations including, but not limited to, Cₘᵢₙ, Cₘₐₓ, and C_{avg} within the broad PR for healthy persons, preferably in mid-range.

In one embodiment, to be exemplified hereafter, the invention provides a method for treating males with hypogonadism. Testosterone replacement for men with primary or secondary hypogonadism is associated with a variety of beneficial effects on fat-free mass, muscle mass and performance, bone mineral density (BMD), mood, energy, and many domains of sexual and cognitive functions. The risks of testosterone replacement therapy are minimal in healthy hypogonadal men.

The physiologic range (PR) of serum testosterone concentrations in healthy young men is wide (300 to 1140 ng/dL), and there is no established consensus on what serum concentrations and/or profile should be the ideal target. Different testosterone-dependent processes have different testosterone dose-response characteristics. Sexual function in men and male mammals has been reported to be maintained when testosterone concentrations are at, or slightly below the lower limit of the PR. In contrast, testosterone effects on fat-free mass and muscle size are reported to be dose- and concentration-dependent. Thus, higher doses and concentrations of testosterone are likely to produce muscle mass accretion greater than that produced with lower doses. However, supraphysiologic doses of testosterone might have adverse effects on plasma lipids and cardiovascular risk.

Therefore, a preferred embodiment at this time of testosterone replacement therapy in males generally is to achieve and maintain the average serum testosterone concentrations within the broad PR for healthy young men, preferably in mid-range.

In particular, two measures, C_{avg} and Cₘᵢₙ of a serum testosterone concentration time course are preferred for predicting therapeutic efficacy and adjusting dosages of the pharmaceutical compositions. These two measures and their corresponding serum testosterone concentrations are Cₘᵢₙ (>300 ng/dL) and C_{avg} (between 300 and 1140 ng/dL).

In particular, the testosterone gel (CP601B) administered in these studies consists of 15% ethanol, 15% isopropanol, 35.1% propylene glycol, 2.5% oleic aid, 2% testosterone, 0.6% CARBOPOL 1382; 0.4% triethanolamine; and 29.4% water (percents as w/w).

Abbreviations

| | |
|---|---|
| BAT | bioactive testosterone |
| b.i.d. | twice daily |
| BMD | bone mineral density |
| BMI | body mass index |
| cm | centimeter |
| CI | confidence interval |
| CRF | case report form |
| CTM | clinical trial material |
| CV | coefficient of variation |
| DHT | 5-α dihydrotestosterone |
| dL | deciliter |
| DRUDP | Division of Reproductive and Urologic Drug Products |
| E₂ | 17-β estradiol |
| EE | efficacy evaluable |
| FSH | follicle stimulating hormone |
| GCPs | Good Clinical Practices |
| g | gram |
| GnRH | gonadotropin releasing hormone |
| ICH | International Conference on Harmonization |
| ITT | intent-to-treat |
| kg | kilogram |
| LH | luteinizing hormone |
| LLN | Lower limit normal |
| LOQ | limit of quantitation |
| MITT | modified intent-to-treat |
| mg | milligram |
| mL | milliliter |
| Mod | Module |
| ng | nanogram |
| NDA | New Drug Application |
| No., N | number |
| PD | pharmacodynamic |
| pg | page |
| PK | pharmacokinetic |
| PR | physiologic range |
| RSD | relative standard deviation |
| SAP | statistical analysis plan |
| SD | standard deviation |
| SHBG | sex hormone binding globulin |
| T | testosterone |
| ULN | upper limit of normal |
| Vol | volume |

Definitions of Terms

| | |
|---|---|
| AUC | area under the serum testosterone concentration-vs-time |
| | curve |
| AUC_{0-24 hr} | AUC from time 0 to 24 hours |
| C₀ | pretreatment serum testosterone concentration, i.e., |
| | immediately prior to gel application |
| C_{avg} | average serum testosterone concentration, determined as |
| | AUC₀₋₂₄ divided by 24 hours |
| Cₘₐₓ | maximum serum testosterone concentration |
| Cₘᵢₙ | minimum serum testosterone concentration |
| Time above | Amount of time (in hours) when serum testosterone |
| 1140 ng/dL | concentration was greater than 1140 ng/dL, as determined |
| | by linear interpolation of concentrations at successive |
| | pairs of time points |
| Time | Amount of time (in hours) when serum testosterone |
| below | concentration was lower than 300 ng/dL, as determined |
| 300 ng/dL | by linear interpolation of concentrations at successive |
| | pairs of time points |
| Time | Amount of time (in hours) when serum testosterone |
| outside | concentration was above or below the physiologic range |
| physiologic | (300-1140 ng/dL), as determined by linear interpolation |
| range | of concentrations at successive pairs of time points |

In a preferred embodiment, the subject is a male with hypogonadism. Hypogonadism is a multi-system syndrome associated with impaired androgen production or action. Androgen deficiency can result from abnormalities of testicular function (primary hypogonadism) or hypothalamic or pituitary regulation of testicular function (secondary hypogonadism) or from impairment of androgen action at the target tissue (androgen resistance). In some embodiments, therefore the subject is a male with classical primary or secondary hypogonadism have serum testosterone concentrations below the lower limit of the PR, i.e., below 300 ng/dL.

Hypogonadism has been treated with administration of exogenous testosterone since the hormone was synthesized some 60 years ago. Most of the published results on testosterone replacement therapy have been focused on replacement in men with primary and secondary hypogonadism.

There is agreement in the literature that testosterone replacement in such subjects is desirable since failure to treat androgen deficiency can lead to serious health consequences. If left untreated, androgen deficiency may contribute to osteoporosis and increased risk of fracture,loss of muscle mass and function, impaired sexual function, lowered mood and energy level, increased fat mass, particularly in the visceral fat compartment, and insulin resistance. Conversely, testosterone replacement in men with classic androgen deficiency syndrome is associated with improvements in body composition, sexual function,sense of well-being and energy, some domains of cognitive function, and an increase in BMD. Thus, the risk-to-benefit ratio of physiologic testosterone replacement therapy in men with classic primary or secondary hypogonadism is highly favorable. In preferred embodiments, the invention is directed toward preventing such conditions and securing such benefits.

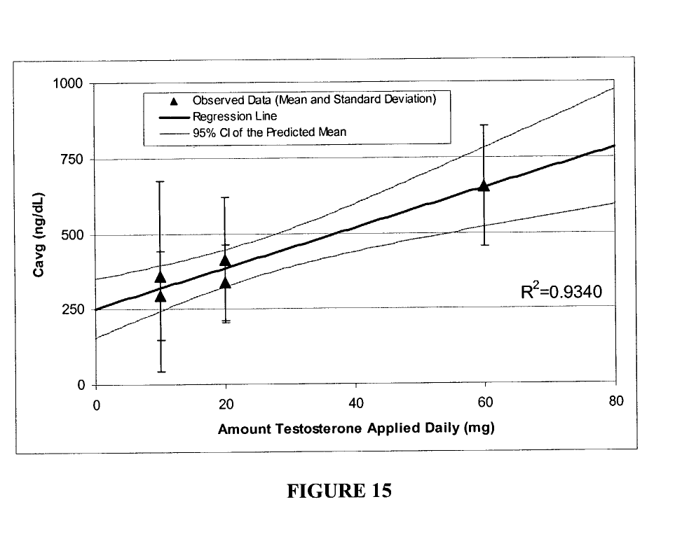

In a preliminary study (see FIG. 15) using CP601, it was found that 3 grams of 2% testosterone gel applied once daily as a divided dose on both thighs, equivalent to 60 mg testosterone applied to the skin, best maintained the serum testosterone concentration in hypogonadal men within the PR of 300-1140 g/dL. CP601 is essentially identical to CP601B except that CARBOPOL™1342 was used rather than CAROBOPOL™1382.

In this dose group, the mean Cavg (652 ng/dL) was significantly higher than the lower limit of the PR, and all subjects had Cavg values >300 ng/dL. Based upon this preliminary study, the predicted percentage of subjects in this population with Cavg values above 300 ng/dL following treatment with 3 grams of 2% testosterone gel once daily was calculated to be 96.3%.

The mean Cₘᵢₙ (383 ng/dL) was significantly higher than the lower limit of the PR; 50.0% of the subjects had Cₘᵢₙ values ≧300 ng/dL, and the predicted percentage of subjects above 300 ng/dL following treatment was calculated to be 69.5%.

The majority of subjects had serum testosterone concentrations between 300 ng/dL and 1140 ng/dL throughout the day.

Although the total subject number enrolled in this initial PK study was small, it was concluded that single daily applications of 3 g CP601 per 300 cm² would provide an adequate starting dose to maintain the serum testosterone concentration in the PR for the large majority of hypogonadal men. CP601 differs from CP601B primarily in the trace amounts of benzene present.

Allowing Adjustment of Each Subject's Dose

A blood-level response relationship is frequently a better indicator of treatment effect than is a dose-response relationship, due to the intersubject variability of absorption, distribution, metabolism and elimination of compounds. Dosage adjustment to attain specific blood levels has been recommended as a means to improve clinical trial outcome, correlation with PD effects and reduction in the number of subjects required to demonstrate effectiveness. In addition, the skin is a rather variable barrier to transdermal delivery of drugs from semi-solid dosage forms into the systemic circulation. For these reasons, the pivotal efficacy study design included a method to allow for dosage adjustment.

Scientific support for selecting the adjustment doses was based on results from an initial pharmacokinetic study as shown in FIG. 15.

For those hypogonadal men whose serum testosterone concentrations would not be properly replaced by the 3 g dose, it was anticipated that adjustment to a lower (e.g., 2 g) or higher (e.g., 4 g) amount of gel would shift their testosterone levels into the desired range.

Therefore a 24-hour serum testosterone concentration profile following 14 days of continuous treatment was used as the basis for maintaining the dose at 3 g (60 mg testosterone applied to the skin) or adjusting the dose to either 2 g (40 mg testosterone applied to the skin) if the testosterone concentrations were too high or 4 g (80 mg testosterone applied to the skin) if the concentrations were too low.

Table 1 provides an overview of the efficacy study. A brief narrative description of the study design and results follows the table.

TABLE 1
An Overview of the Efficacy Study

| Study Design | Open-label, non-vehicle-controlled |
|---|---|
| Treatment Regimen (Dose) | 3 g of CP601B (60 mg T applied to the |
| | skin) once daily for 182 days; the dose |
| | could be modified at Day 29 (increased |
| | to 4 g gel [80 mg T/day] or decreased to |
| | 2 g gel [40 mg T/day]) depending on |
| | serum T concentration measured on |
| | Day 14. |
| Subjects Enrolled | 204^{a} |
| % Subjects by Sex (M/F) | 100%/0% |
| % Subjects by Race (C/B/O) | 84.1%/13.4%/2.5% |
| Mean Age in Years (Range) | 53.2 (19-74) |

KEY: M = male, F = female, C = Caucasian, B = Black, O = Other
^{a}Three subjects enrolled twice into the study.

Study Design

The study was conducted to determine the effectiveness of transdermal CP601B in keeping testosterone concentrations of hypogonadal men within the PR. Men aged 18-75 years were eligible for participation if they had primary or secondary hypogonadism, defined as a serum testosterone concentration <250 ng/dL in a single blood sample or <300 ng/dL in two consecutive samples obtained at least one week apart. Subjects were to discontinue use of any current testosterone medication prior to entering the study.

Study was a multicenter, open-label, non vehicle-controlled trial in which subjects applied CP601B to their inner thighs once daily. All subjects began treatment using 3 g of gel (60 mg T applied to the skin). A 24-hour pharmacokinetic profile was obtained after application of the first dose and on Day 14 (±2). Depending on the testosterone concentrations measured on Day 14, subjects were instructed on Day 28 to either decrease their dose to 2 g gel (40 mg T) if the testosterone concentrations were too high, increase to 4 g gel (80 mg T) if the serum testosterone concentrations were too low, or remain on the 3 g dose. Subjects continued once daily application of the dose assigned on Day 28 for the remainder of the study. Follow-up visits occurred on Days 42/56, 70, 98, 140, and 182. The 24-hour pharmacokinetic profile was repeated on Day 42 (±4) for subjects who continued using 3 g gel after the Day 28 visit, and on Day 56 (±4) for subjects who began using 2 or 4 g gel on Day 29. A final 24-hour pharmacokinetic profile was obtained on Day 182. Subjects who completed the study through Day 182 could participate in a 12-month extension study to assess long-term safety. This report includes results from the 6-month study only; results from the extension study are reported separately.

The effectiveness of CP601B was evaluated through measurements of serum testosterone concentrations. Bioactive testosterone (BAT), 5-αdihydrotestosterone (DHT), sex hormone-binding globulin (SHBG), estradiol (E₂), follicle stimulating hormone (FSH), and luteinizing hormone (LH) were also measured. In a subset of subjects who had not been treated previously with testosterone products and who were evaluated at sites with the appropriate equipment, BMD of the hip and the lumbar spine was measured at baseline and after 182 days of treatment.

The following PK parameters for testosterone were computed for each subject using a model-independent approach: minimum, average, and maximum serum concentrations during the 24-hour period following dose application (Cₘᵢₙ, C_{avg}, and Cₘₐₓ, respectively). The primary efficacy endpoint was the proportion of efficacy evaluable subjects with both Cₘᵢₙ and C_{avg} within the PR (300 to 1140 ng/dL) on Day 42/56, the primary efficacy day. The proportion of subjects who fulfilled these criteria was compared to an historical rate of 35% using a test of noninferiority with an allowable difference (delta), or noninferiority margin, of 15%. The test of noninferiority was conducted by computing a 95% confidence interval (CI) and comparing the lower confidence bound to 20%. The primary endpoint was considered met if the lower bound of the 95% CI of the proportion of efficacy evaluable subjects with both Cₘᵢₙ and C_{avg} within the PR was above the 20% non-inferiority margin. Other efficacy variables included the proportion of subjects with C_{avg} within the PR on Day 42/56, the proportion of subjects who met that criterion and also had serum testosterone concentrations ≧300 ng/dL for at least 80% of the dosing interval on Day 42/56, the proportion of subjects with serum testosterone concentrations within the PR for at least 80% of the dosing interval, summary statistics for PK parameters, and the correlation of PK results with BMI and age. Similar analyses were repeated for the secondary efficacy day, Day 182. This study was conducted in accordance with the “Recommendations Guiding Medical Doctors in Biomedical Research Involving Human Subjects” contained in the Declaration of Helsinki and in compliance with Good Clinical Practices (GCPs) as described in the International Conference on Harmonization (ICH) Guidance for GCP. Subjects provided written consent to participate in the study after having been informed about the nature and purpose of the study, participation/termination conditions, and risks and benefits of treatment. Informed consent was obtained before any study-related screening procedures were performed.

Choice of Populations in Efficacy Analyses

The statistical analysis plan (SAP) for the study identified three populations for efficacy analyses: intent-to-treat (ITT), modified intent-to-treat (MITT), and efficacy evaluable (EE). Dosage adjustment is frequently necessary to establish serum testosterone levels of hypogonadal men in the PR due to interindividual variation in skin permeability, testosterone clearance, and other factors. The purpose of the MITT population is to appropriately test the effect of the dosage adjustment featured in this protocol. Because the ITT population is defined (see below) as all subjects who received a minimum of one dose of study drug, it includes any subject who discontinues from the study prior to the dosage adjustment and/or prior to obtaining PK profiles at the adjusted dose. The ITT population is not best suited for efficacy assessments in this trial because it includes the subset of discontinued subjects who are lacking the very data necessary to evaluate the success of the dosage adjustment. Therefore, to establish whether the dosage adjustment was successful, a MITT population was defined, which is composed of the same subjects as in the ITT population excluding those subjects who did not reach the dosage assessment period of the trial (see definition below). It is this MITT population that will be the most appropriate population to test the success of the dosage adjustment scheme that is at the core of this clinical trial.

Specifically, the analysis populations are defined as follows:

ITT population:

All subjects who applied a minimum of one dose of study drug.

MITT Populations:

Day 42/56 MITT (For efficacy analyses on the primary efficacy day, Day 42/56): All subjects who applied a minimum of one dose of study drug, remained in study up to Day 42/56 and had more than one PK sample obtained during the 24-hour pharmacokinetic profile on Day 42/56;

Day 182 MITT (For efficacy analyses of the secondary efficacy day, Day 182): All subjects who applied a minimum of one dose of study drug, remained in study up to Day 182 and had more than one PK sample obtained during the 24-hour pharmacokinetic profile on Day 182;

EE Populations:

These populations (i.e. EE populations on Days 42/56 and 182) were much more conservative subsets of subjects who met the key protocol study requirements. Both Day 42/56 EE and Day 182 populations shared the following common requirements

applied a minimum of one dose of study drug;

met the criterion for hypogonadism based upon a serum testosterone concentration(s) at screening (see below);

had an adjustment to study drug dose in accordance with the protocol-specified criteria, including medical monitor adjustments;

did not use prohibited concomitant medications;

Specifically, for efficacy analyses on the primary efficacy day (Day 42/56), additional requirements were included:

remained in study up to Day 42/56 at least;

had no more than one missing time point on Day 1 and full 24-hour pharmacokinetic profiles (without one single missing sample) on Days 14 and 42/56;

used at least 70% but no more than 130% of the prescribed amount of study drug between Day 1 and Day 42/56.

For efficacy analyses of the secondary efficacy day (Day 182), following are the additional requirements:

remained in study up to Day 182;

had no more than one missing time point on Day 1 and full 24 hour pharmacokinetic profiles without one sample missing of Day 14 and Day 182.

used at least 70% but no more than 130% of the prescribed amount of study drug for the entire study.

Two-hundred-and-four subjects were enrolled and used at least one dose of CP601B (ITT population). Of these, 163 completed the 24-hour pharmacokinetic profile on Day 42/56 and were included in the MITT population for Day 42/56. Eighty-nine subjects met the criteria for the EE population for Day 42/56. For analyses of Day 182 results, the ITT, MITT, and EE populations included 201, 146, and 84 subjects, respectively

All subjects applied 3 g of gel (60 mg T) each day from Day 1 through Day 28. Starting on Day 29, 32 subjects of the ITT population had their dose reduced to 2 g of gel (40 mg T) and 69 subjects had their dose increased to 4 g of gel (80 mg T) for the rest of the study. Of the remaining 100 subjects that were not dose adjusted, 24 discontinued before or on Day 28, and 76 subjects continued using 3 g of gel (60 mg T) for the remainder of their participation in the study. The mean duration of exposure was 148.5±64.02 days. The duration of exposure was similar among those taking final assigned doses of 2 g (173.1 days) and 4 g gel (168.7 days) and was lower for those taking 3 g (122.3 days).

The average testosterone concentration (±standard deviation) on entry into study was 181.0±88.61 ng/dL for the Day 42/56 EE population, 202.3±121.73 ng/dL for the Day 42/56 MITT population and 204.01±118.68 ng/dL for the ITT population. Within 30 minutes of the first application of CP601B, the testosterone concentrations increased in 44.2% of the Day 42/56 MITT subjects (72 of 163). By 4 hours after the dose, concentrations were above 300 ng/dL in 64.8% of these subjects. After only one dose of CP601B had been applied (3 g gel on Day 1), the mean C_{avg} for the 163 MITT subjects reached 360.3±155.59 ng/dL, an average increase of 158 ng/dL. A once-daily dose of CP601B provided continuous testosterone replacement through the entire 24-hour dosing interval.

After 14 days of daily treatment with a fixed dose of CP601B (3 g), subjects could be individually assessed and assigned to one of three groups depending on whether their dose of CP601B was to be increased to 4 g, decreased to 2 g, or kept the same. Following dose adjustment, the mean testosterone concentration-time curves were almost superimposed for all three groups on Days 42/56 and 182.

CP601B dose adjustment resulted in the expected and highly significant changes in Cₘᵢₙ, C_{avg} and Cₘₐₓ in the three final dose groups. Large differences in these parameters, particularly in C_{avg} and Cₘₐₓ, which were apparent on Day 14, disappeared following dose adjustment. Dose adjustment of CP601B was highly effective in decreasing testosterone concentrations and the corresponding PK parameters that were judged too high, or increasing those that were too low.

The primary endpoint (Cₘᵢₙ and C_{avg} within the PR) was met in all three study populations (EE, MITT and ITT) on both the primary (Day 42/56) and the secondary (Day 182) efficacy days. The primary endpoint on primary efficacy day (43/56) was met in 41.7% of subjects of the Day 42/56MITT population (68 of 163 subjects with C_{avg} and Cₘᵢₙ within the PR). The lower bound of the 95% CI (34.1%) was higher than the 20% non-inferiority margin for the 35% historic point estimate (with an allowable delta of 15%). Similar results were found for the Day 42/56 EE population. For the ITT population, 33.8% subjects had both Cmin and Cavg within the PR, with the lower bound of the 95% CI (27.3%) still well above the non-inferiority margin.

The secondary endpoint (C_{avg} within the PR on Day 42/56) was met by the Day 42/56 and Day 182 EE and MITT populations. Specifically, 92% of subjects (150 of 163) in the Day 42/56 MITT population had C_{avg} within the PR. The lower bound of the 95% CI (86.5%) was higher than the 65% non-inferiority margin for the 80% historic point estimate with an allowable delta of 15%. Similar results were found with the EE population. In the ITT population, 75% of the subjects had Cavg values within the PR and the lower bound of the 95% CI (68.8%) was above the non-inferiority margin.

Subjects with no measurable endogenous testosterone had increases in testosterone concentration (both Cₘᵢₙ and C_{avg}) as early as Day 1 with only 3 g of gel. At steady-state and after the dose was properly adjusted in all subjects, C_{avg} of about 500 ng/dL was achieved. Additionally, rates of success on the primary and secondary endpoints in this challenging population were similar to those in the other populations.

Serum DHT concentrations increased from a mean of 18.5 ng/dL at baseline to 78.0 ng/dL on Day 14, and 86.2 ng/dL on Day 42/56 and 86.4 ng/dl on Day 182 in the Day 42/56 MITT subjects. The DHT to testosterone ratio increased from 0.12 at baseline to 0.21 on Day 14, 0.22 on Day 42/56 and 0.22 on Day 182 in the same group of men. DHT to testosterone ratios were numerically higher on Day 182 than at baseline; these differences did not achieve statistical significance. Serum BAT levels also increased over time but remained within the normal range for males. Treatment with CP601B was associated with a significant increase in E₂ concentrations from a mean of 1.6 ng/dL at time 0 Day 1 to 3.6 ng/dL on Day 42/56, and 3.4 ng/dL on Day 182 of the study. The mean estradiol to testosterone ratio remained unchanged throughout the study. Mean values for SHBG did not change significantly over the course of 182 days of treatment with CP601B. Levels of FSH and LH decreased modestly, as expected.

Following six months of CP601B treatment, a significant 2% increase in hip and spine BMD was observed in subjects who had never used testosterone replacement products.

C_{avg} and Cₘₐₓ were correlated inversely with BMI and weight before dose adjustment (Day 14). Following dose adjustment, there was no correlation in the 2 and 3 g groups, but the correlation remained in the 4 g group. Additionally, it was determined that subjects with BMI over 45 kg/m² were highly likely to be assigned to a 4 g gel dose. The primary and secondary study endpoints on Day 42/56 in the challenging populations of 33 subjects with BMI≧36 kg/m² and in a subset of 9 subjects with BMI≧45 kg/m² were similar to those observed in the main ITT population.

There was no correlation between age and any of the PK parameters. CP601B was equally effective in treating younger subjects (<55 years) and older subjects (≧55 years).

The serum testosterone concentration measured 2 hours after CP601B administration on Day 14 was highly correlated with both C_{avg}, a good surrogate marker for efficacy, and Cₘₐₓ, a surrogate marker for safety. In some embodiments therefore, the blood sample for measurement is taken about two hours after CP601B application and this value is used to guide dosage adjustments.

Efficacy results in the EE and MITT populations were similar, confirming that results obtained in the smaller EE population could be generalized to a less restrictively defined hypogonadal population.

Demographics and baseline characteristics of the ITT, Day 42/56 MITT, and Day 42/56 EE populations were similar as shown in Table 2. The age range of the subjects in the ITT population was between 19 and 74 years (mean, 53.2±11.5 years). The majority of subjects were Caucasian (84.1%). Most subjects (75.6%) had used testosterone replacement products before entering the study. The mean serum testosterone level of these subjects was 204.0±118.7 ng/dL at entry (Day 1, hour 0).

The demographic and baseline characteristics of the Day 42/56 MITT and EE populations were similar to those of the ITT population. The age range was 19 to 74 years (mean, 53.2 years for the MITT and 51.8 years for the EE). The majority of subjects were Caucasian (MITT: 82.8%; EE: 86.5%). Most subjects (76% of both groups) had used testosterone replacement products before entering the study. The mean serum testosterone level at baseline for the MITT population (202.3±121.7 ng/dL) was similar to the ITT population; baseline mean testosterone level was about 10% lower for the EE population (181.0±88.6 ng/dL). Median results for the three groups, however, were within 5% of each other, ranging from 193 to 202 ng/dL.

TABLE 2
Demographic and Baseline Characteristics

| | Day 42/56 | Day 42/56 | Day 42/56 |
|---|---|---|---|
| | ITT | MITT | EE |
| | (N = 201^{a}) | (N = 163) | (N = 89) |
| AGE | | | |
| <55 | 112 (55.7) | 92 (56.4) | 53 (59.6) |
| >=55 | 89 (44.3) | 71 (43.6) | 36 (40.4) |
| N | 201 | 163 | 89 |
| MEAN ± SD | 53.2 ± 11.47 | 53.2 ± 11.22 | 51.8 ± 11.52 |
| MEDIAN | 53.0 | 53.0 | 53.0 |
| RANGE | 19.0-74.0 | 19.0-74.0 | 19.0-74.0 |
| RACE | | | |
| CAUCASIAN | 169 (84.1) | 135 (82.8) | 77 (86.5) |
| BLACK | 27 (13.4) | 24 (14.7) | 10 (11.2) |
| ASIAN | 1 (0.5) | 1 (0.6) | 1 (1.1) |
| HISPANIC | 4 (2.0) | 3 (1.8) | 1 (1.1) |
| NATIVE | 0 (0.0) | 0 (0.0) | 0 (0.0) |
| AMERICAN | | | |
| OTHER | 0 (0.0) | 0 (0.0) | 0 (0.0) |
| WEIGHT (KG) | | | |
| N | 201 | 163 | 89 |
| MEAN ± SD | 102.2 ± 21.56 | 102.5 ± 22.42 | 101.2 ± 22.11 |
| MEDIAN | 99.9 | 99.9 | 98.1 |
| RANGE | 56.8-202.9 | 56.8-202.9 | 64.1-202.9 |
| HEIGHT (CM) | | | |
| N | 201 | 163 | 89 |
| MEAN ± SD | 179.8 ± 8.82 | 179.7 ± 9.03 | 180.1 ± 8.69 |
| MEDIAN | 180.3 | 179.1 | 180.3 |
| RANGE | 141.2-213.4 | 141.2-213.4 | 141.2-200.7 |

BODY MASS INDEX (KG/M²)

| | | | |
|---|---|---|---|
| N | 201 | 163 | 89 |
| MEAN ± SD | 31.6 ± 6.11 | 31.7 ± 6.30 | 31.2 ± 6.35 |
| MEDIAN | 30.4 | 30.4 | 29.9 |
| RANGE | 16.5-54.5 | 16.5-54.5 | 20.3-54.5 |

PRIOR TESTOSTERONE SUPPLEMENT USE

| | | | |
|---|---|---|---|
| YES | 152 (75.6) | 124 (76.1) | 68 (76.4) |
| NO | 49 (24.4) | 39 (23.9) | 21 (23.6) |

BONE MINERAL DENSITY-LUMBAR SPINE (GHA/CM²)^{b}

| | | | |
|---|---|---|---|
| N | 42 | 35 | 20 |
| MEAN ± SD | 1.1 ± 0.20 | 1.1 ± 0.20 | 1.1 ± 0.23 |
| MEDIAN | 1.1 | 1.1 | 1.1 |
| RANGE | 0.6-1.8 | 0.6-1.8 | 0.7-1.8 |
| MISSING | 159 | 128 | 69 |

BONE MINERAL DENSITY-HIP (GHA/CM²)^{b}

| | | | |
|---|---|---|---|
| N | 43 | 36 | 20 |
| MEAN ± SD | 1.1 ± 0.19 | 1.1 ± 0.19 | 1.1 ± 0.18 |
| MEDIAN | 1.1 | 1.1 | 1.1 |
| RANGE | 0.7-1.5 | 0.7-1.5 | 0.7-1.4 |
| MISSING | 158 | 127 | 69 |

SERUM TESTOSTERONE LEVEL (NG/DL)^{c}

| | | | |
|---|---|---|---|
| N | 200 | 163 | 89 |
| MEAN ± SD | 204.0 ± 118.68 | 202.3 ± 121.73 | 181.0 ± 88.61 |
| MEDIAN | 202.0 | 201.0 | 193.0 |
| RANGE | 25.0-906.0 | 25.0-906.0 | 25.0-359.0 |
| MISSING | 1 | 0 | 0 |

^{a}For the purpose of display in this table, demographic and baseline data from the first enrollment were used for the three subjects who enrolled twice.
^{b}BMD measurements were obtained on subjects who had not used testosterone replacement products previously.
^{c}Baseline is PK Day 1, hr 0.
NOTE: Values represent number (%) of subjects unless otherwise indicated.

Table 3 provides completion/withdrawal information for the ITT and the Day 42/56 MITT and EE populations. Of the 163 Day 42/56 MITT subjects, 145 (89%) completed the 6-month study, 18 (11%) withdrew during the study. The most common reason for premature withdrawal was adverse events (n=8, 4.9%).

TABLE 3
Study Completion/Withdrawal Information

| | | | Day 42/56 | | Day 42/56 | |
|---|---|---|---|---|---|---|
| | ITT^{a} | | MITT^{a} | | EE^{a} | |
| | N | (%) | N | (%) | N | (%) |
| Number of Subjects | 201^{b} | | 163 | | 89 | |
| Enrolled and Received | | | | | | |
| CTM | | | | | | |
| Number of Subjects | 145 | (72.1) | 145 | (89.0) | 82 | (92.1) |
| Completing 6-month | | | | | | |
| Study | | | | | | |
| Number of Subjects | 56 | (27.9) | 18 | (11.0) | 7 | (7.9) |
| Who Prematurely | | | | | | |
| Withdrew From 6- | | | | | | |
| month Study | | | | | | |
| Reason for Premature | | | | | | |
| Withdrawal | | | | | | |
| Adverse Event | 33^{c} | (16.4) | 8 | (4.9) | 3 | (3.4) |
| Protocol Violation | 4 | (2.0) | 0 | (0.0) | 0 | (0.0) |
| Subject | 7 | (3.5) | 5 | (3.1) | 2 | (2.2) |
| Non-Compliance | | | | | | |
| Subject Choice | 7 | (3.5) | 2 | (1.2) | 0 | (0.0) |
| Lost to Follow-Up | 1 | (0.5) | 1 | (0.6) | 1 | (1.1) |
| Other^{d} | 4 | (2.0) | 2 | (1.2) | 1 | (1.1) |
| ^{a}Percentages based on total number of subjects with completion/withdrawal information. | | | | | | |
| ^{b}Three subjects enrolled twice. Subject 014-130 (discontinued after 17 days) reenrolled as Subject 014-143 (completed study). Only his first enrollment was included in the analysis of efficacy. The other two subjects who entered twice did not complete the study. Summaries of demography and the incidence of adverse events are based on a total of 201 subjects, because demographic data from the first enrollment were used, and adverse | | | | | | |
| # events reported during each enrollment were reported as occurring in one individual. Other safety tables, such as the displays of extent of exposure, vital signs, and laboratory results, report the findings from each subject enrollment individually (total 204 subjects). | | | | | | |

^{c}Includes 33 subjects who discontinued due to treatment-emergent adverse events (one subject [002-106] withdrew on his 2^{nd} enrollment, and is therefore not represented in the ITT column) and one subject (014-131) who discontinued due to a non-treatment-emergent adverse event
^{d}Subjects who withdrew for “other” reasons included:
010-104-ITT, -SAFE: patient did not inform us at screen of his diagnosis of sleep apnea; sponsor requested drop.
011-114-ITT, -MITT, -EE, and -SAFE: study closed at site.
014-130-ITT, -SAFE: unable to keep week 2 appointment due to new job and holidays.
015-100-ITT, -MITT, -SAFE: sponsor request due to coming surgery

Comparison of Efficacy Results of All Studies

Efficacy Endpoints and Analyses

The primary endpoint was defined as the proportion of subjects with both C_{avg} and Cₘᵢₙ within the PR on Day 42/56. Additionally, there were three secondary endpoints:

1. The proportion of subjects with C_{avg} within the PR on Day 42/56;

2. The proportion of subjects with both C_{avg} and Cₘᵢₙ within the PR on Day 182; and

3. The proportion of subjects with C_{avg} within the PR on Day 182.

A number of other analyses, which were also defined in the statistical analysis plan (SAP), are also reported:

4. Analysis of the proportion of subjects with C_{avg} within the PR and other concentrations >300 ng/dL for more than 80% of the dosing interval;

5. Analysis of the proportion of subjects with testosterone concentrations within the PR for more than 80% of the dosing interval;

6. Analysis of other hormone (DHT, E₂, FSH, LH) and SHBG concentrations and ratios of DHT/T and E₂/T;

7. Analysis of changes in BMD; and

8. Analysis of the effect of age on efficacy endpoints.

Finally, a number of additional analyses were added to those originally described in the SAP to better characterize the testosterone pharmacokinetics in the hypogonadal study population and to further refine the efficacy assessments. These included the following:

9. Analysis of testosterone concentrations on entry into the study;

10. Analysis of mean testosteone concentration-time profiles;

11. Analysis of individual key PK parameters Cₘᵢₙ, C_{avg}, and Cₘₐₓ;

12. Analysis of time testosterone concentrations were maintained within the PR;

13. Correlation between PK parameters and BMI, weight, and age;

14. Calculation of CP601B relative systemic bioavailability;

15. Subgroup analysis of subjects with no measurable endogenous testosterone;

16. Subgroup analysis of subjects with BMI≧36kg/m² and ≧45 kg/m²;

17. Identification of a single blood sampling time point for use in dose adjustment.

All hormone concentrations referred to in this section pertain to those measured in serum.

Characterization of Testosterone Pharmacokinetics Following Application of CP601B in Hypogonadal Subjects

Pretreatment Testosterone Concentrations: Day 1 C₀ and 24-Hour Baseline

Day 1 C₀

Male subjects were to be enrolled into this study only if they had one serum testosterone concentration below 250 ng/dL or two consecutive measurements below 300 ng/dL at least 7 days apart. The average testosterone concentration (±SD) on entry into study (Day 1 C₀, defined as the Day 1 Hr 0 testosterone concentration) was 181.0±88.6 ng/dL for the Day 42/56 EE population, 202.3±121.7 ng/dL for the Day 42/56 MITT population, and 204.0±118.7 ng/dL for the Day 42/56 ITT population (Table 3). Summary statistics for Day 1 C₀ are shown by final dose group in Table 4 for the Day 42/56 MITT subjects. Day 1 C₀ values were similar across the three dose groups, indicating that final dose assignment was independent of pretreatment testosterone concentrations.

TABLE 4
Summary Statistics of Testosterone Concentration on Entry into Study (Day 1 C₀):
Day 42/56 MITT Population

| | | Final Dose Group^{a} | | | |
|---|---|---|---|---|---|
| | | 2 g gel | 3 g gel | 4 g gel | |
| | Statistic | (40 mg T)^{b} | (60 mg T)^{b} | (80 mg T)^{b} | All |
| Day 1 C₀ | N | 29 | 71 | 63 | 163 |
| (ng/dL) | Mean ± SD | 184.6 ± 106.07 | 209.4 ± 135.23 | 202.4 ± 113.07 | 202.3 ± 121.73 |
| | Median | 167.0 | 210.0 | 198.0 | 201.0 |
| | Range | 25.0-372.0 | 25.0-906.0 | 25.0-675.0 | 25.0-906.0 |

^{a}Subjects in the 2 g and 4 g groups received 3 g daily for the first 28 days; subjects in the 3 g group received 3 g daily for all 182 days.
^{b}For each gram of gel, 20 mg testosterone was applied to the skin.
KEY: T = testosterone

24-Hour Pretreatment Testosterone Concentration-Time Profile

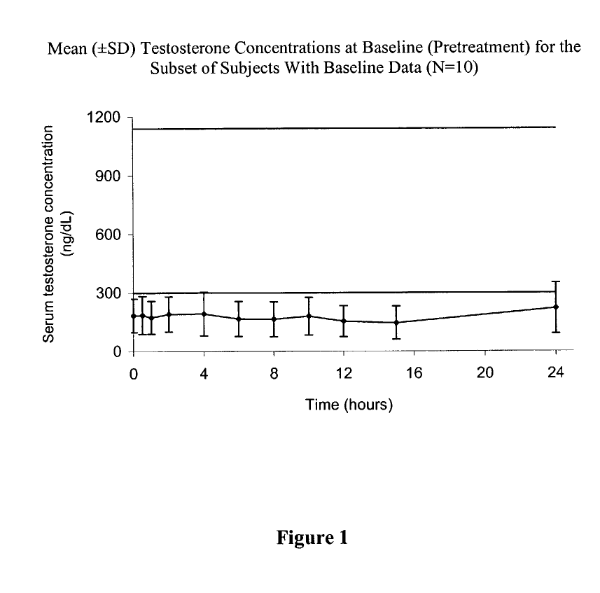

24-hour testosterone profiles were also determined before any CP601B treatment (between screening and Day 1) in a subset of 10 subjects. The mean pretreatment testosterone concentration profile in these 10 subjects is shown in FIG. 1 and summary statistics are provided in Table 5. As expected from untreated hypogonadal men and from the inclusion criteria regarding screening testosterone concentrations, the mean profile was well below 300 ng/dL, the lower end of the PR, with a mean C_{avg} of 176±92 ng/dL. There were no overall discernible diurnal variations of the testosterone concentrations in these subjects.

TABLE 5
Descriptive Statistics for Cₘᵢₙ, C_{avg}, and Cₘₐₓ at Baseline
(Pretreatment) for the Subset of Subjects with Baseline Data (N = 10)

| | | |
|---|---|---|
| Cₘᵢₙ | Mean (SD) | 129.4 (69.02) |
| | Median | 148.5 |
| | Range | 25.0-212.0 |
| C_{avg} | Mean (SD) | 175.8 (91.80) |
| | Median | 225.1 |
| | Range | 27.6-254.5 |
| Cₘₐₓ | Mean (SD) | 239.6 (118.21) |
| | Median | 282.5 |
| | Range | 50.0-398.0 |

Mean Testosterone Profiles On Days 1,14, 42/56 and 182 and Relationship to Final Dose Groups

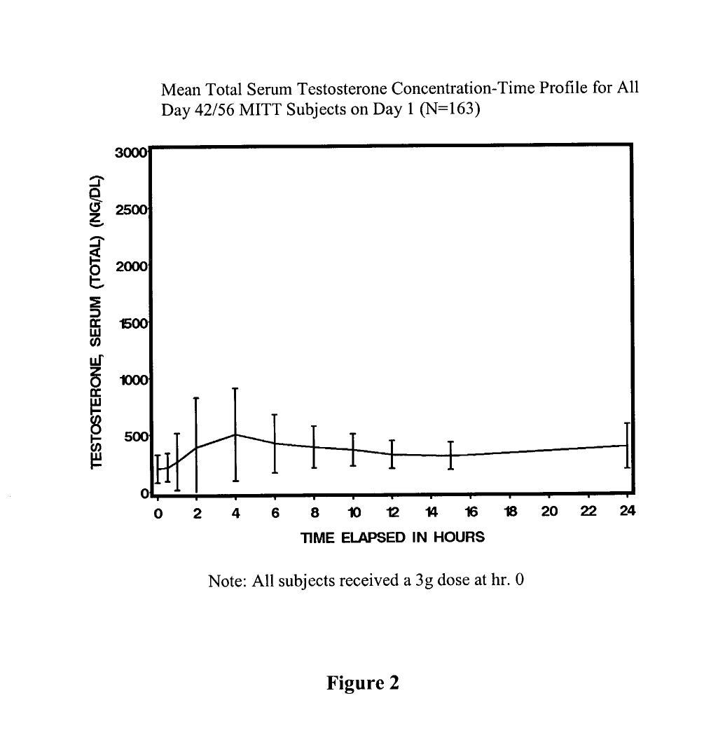

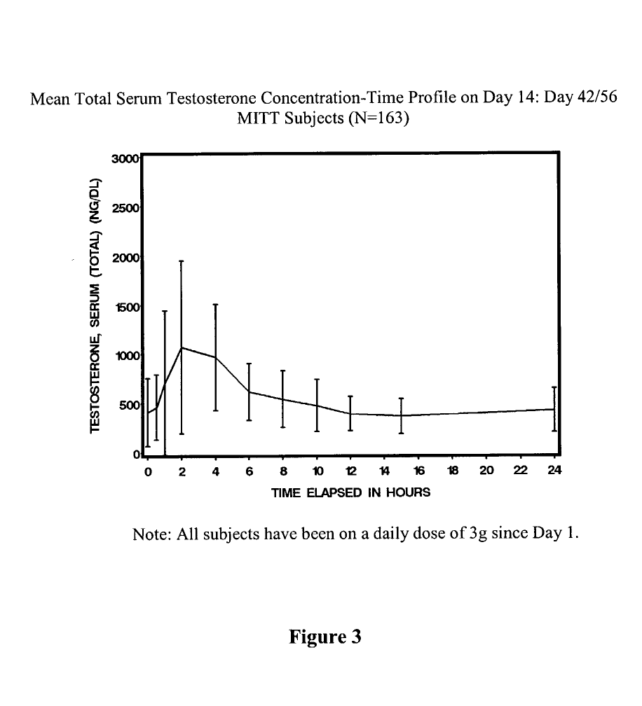

For the first 28 days of the study, all subjects were to apply 3 g of CP601B (60 mg testosterone) once daily to the inner thighs. The mean testosterone concentration-time profiles (±SD) on the two PK days when all subjects (MITT population) received the same 3 g dose of gel (Day 1 and Day 14) are illustrated on FIGS. 2 and 3.

Day 1

Within 30 minutes of the first application of 3 g CP601B, the testosterone concentrations increased in 44.2% (72/163) of the Day 42/56 MITT subjects. By 4 hours after the dose, concentrations were above 300 ng/dL in 64.8% of these subjects. Results in the MITT and ITT population were similar.

Day 14

Each subject's individual PK profile obtained on Day 14 was used to determine if dose adjustment was necessary on Day 28, in accordance with preset rules. Based upon individual testosterone concentration time profiles, dose adjustment was necessary in some subjects to compensate for interindividual variability in skin permeability and other factors and to attain and best maintain each subject's testosterone concentrations within the PR.

Per study protocol, subjects with a Cₘᵢₙ above 400 ng/dL and a Cₘₐₓ greater than 1000 ng/dL on Day 14 were instructed to decrease the applied gel dose from 3 g (60 mg testosterone) to 2 g (40 mg testosterone). Conversely, subjects with Cₘᵢₙ values below 300 ng/dL and Cₘₐₓ below 1000 ng/dL on Day 14 were instructed on Day 28 to start applying 4 g of gel (80 mg testosterone) on Day 29 instead of the initial dose of 3 g (60 mg testosterone). Subjects with both Cₘᵢₙ and Cₘₐₓ within the PR were instructed to remain on the initial dose (3 g) of gel. Thus, starting on Day 29, and based on the Day 14 PK profile, three subgroups of subjects were created as follows:

2 g (40 mg T) Final Dose Group

Subjects who applied 3 g gel (60 mg testosterone) from Day 1 through Day 28 and whose dose was decreased to 2 g gel (40 mg testosterone) for Days 29-182.

3 g (60 mg T) Final Dose Group

Subjects who applied 3 g gel (60 mg testosterone) from Day 1 through Day 182, i.e., whose gel dose was not changed during the course of the study.

4 g (80 mg T) Final Dose Group

Subjects who applied 3 g gel (60 mg testosterone) from Day 1 through Day 28 and whose dose was increased to 4 g gel (80 mg testosterone) for Day 29-182.

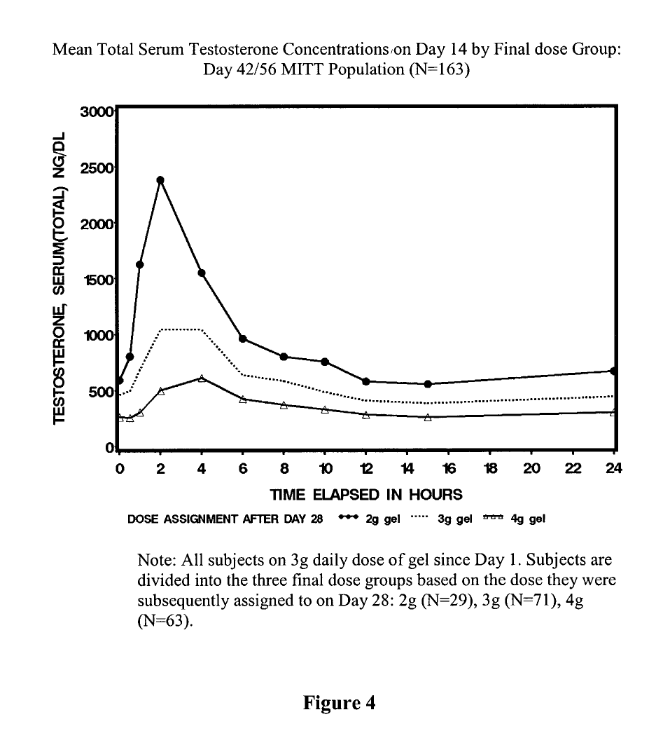

To further characterize these three subgroups, the mean testosterone profile for all subjects on Day 14 (FIG. 3) was divided into three mean profiles corresponding to the three final dose groups. The overlay of the three mean testosterone concentration-time profiles is shown in FIG. 4 for the Day 42/56 MITT population.

As expected from the subgroup definitions, the highest mean testosterone concentration-time profile on Day 14 was that of the subjects assigned to a decrease in dose (2 g group), followed by that of the subjects remaining on the initial 3 g gel dose. Subjects assigned to receive an increase in dose from 3 g to 4 g of gel on Day 28 (4 g group) were those with the lowest mean testosterone profile.

Days 42/56 and 182

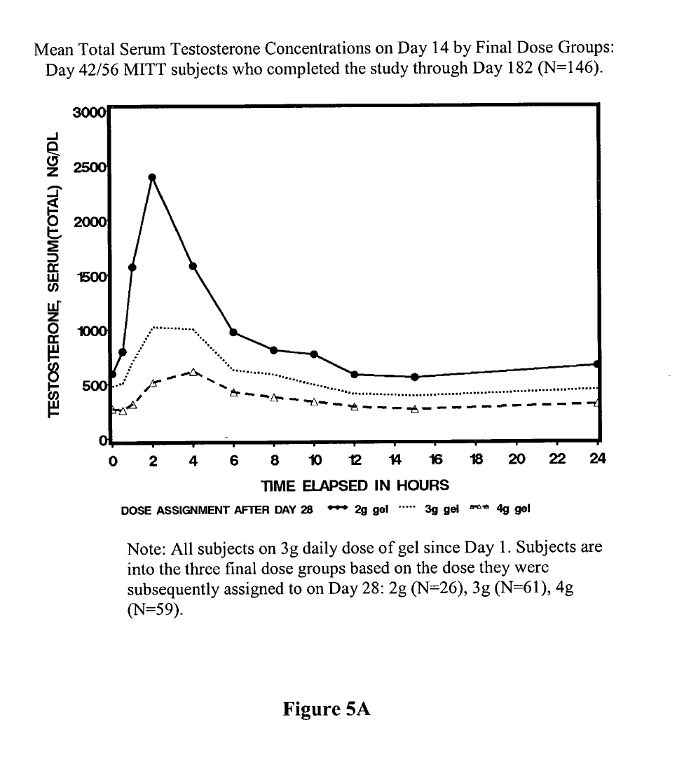

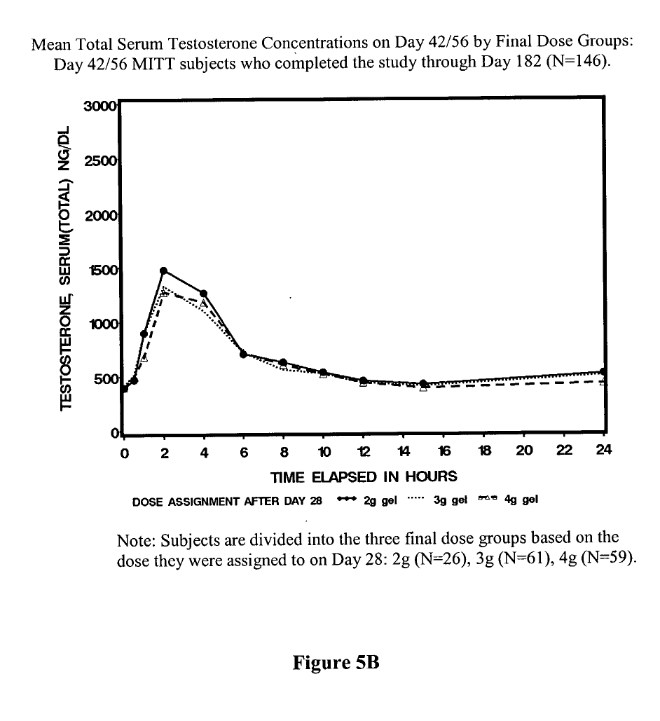

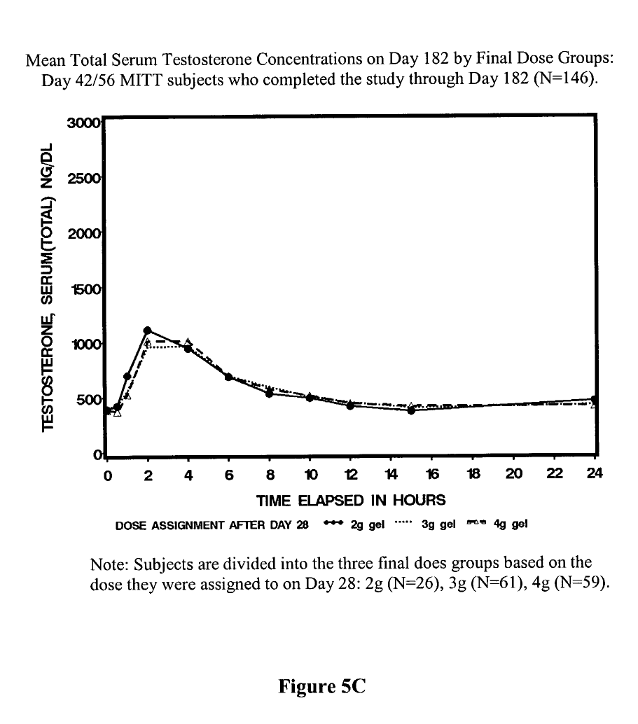

The effect of adjusting the daily CP601B dose on the mean testosterone concentration-time profiles is shown in FIGS. 5A-5C for Day 14 (before dose adjustment), Day 42/56 and Day 182 (after dose adjustment) for all Day 42/56 MITT subjects who completed the study through Day 182 (146 subjects).

As discussed above, before dose adjustment, the three mean PK profiles on Day 14 were clearly different among the three final dose groups. Following dose adjustment, the profiles were nearly the same in the three subgroups, both on Day 42/56 and Day 182.

In summary, the pharmacokinetic results following 14 days of daily application of 3 g CP601B provided the basis for individualized dose requirements and allowed assignment to one of three final dose groups: (increase to) 4 g, (decrease to) 2 g, or 3 g (unchanged). As a result of dose adjustment, the mean testosterone concentration-time curves were similar for all three groups on both Days 42/56 and 182. Dose adjustment with CP601B is thus an effective means to compensate for interindividual variability and bring subjects within a similar range of testosterone concentrations. Therefore, the results obtained after Day 28 could be pooled without regard to the dose applied. This further supports evaluating the success rates for study endpoints using the overall study population rather than each of the 3 dose groups (Sanathanan L P, Peck C C Control Clin Trials 1992;51:465-73; Peck C C Barr W H Benet L Z Collins J Desjardins R E Furst D E Harter J G Levy G Ludden T Rodman J H et al Clin Pharmacol Ther 1992; 51:465-73.

Cₘᵢₙ, C_{avg}, Cₘₐₓ: Descriptive Statistics on Days 1, 14, 42156, and 182

In the section above, PK results were discussed in terms of mean testosterone concentration-time profiles. In this section PK results are discussed in terms of the individual PK parameters—Cₘᵢₙ, C_{avg}, and Cₘₐₓ.

Descriptive statistics for testosterone Cₘᵢₙ, C_{avg}, and Cₘₐₓ on each of the pharmacokinetic evaluation days are presented in Table 6.1 for the Day 42/56 EE population and in Table 6.2 for the Day 42/56 MITT population. The results are shown for all subjects and for subgroups based on the final assigned dose (2, 3, and 4 g CP601B gel, corresponding to 40, 60, or 80 mg testosterone applied to the skin, respectively).

After only one dose of CP601B had been applied (3 g gel), the mean C_{avg} for the 163 Day 42/56 MITT subjects reached 360.3±155.6 ng/dL, an average increase of about 160 ng/dL from the testosterone concentration on entry in the study (Day 1 C₀=202.3±121.7 ng/dL). Mean values for Cₘᵢₙ, C_{avg}, and Cₘₐₓ all increased from Day 1 to Day 14 when all subjects were receiving 3 g gel (60 mg testosterone). Results in the EE population were similar to those obtained in the MITT population. Thus, conclusions derived from the smaller, more restrictively defined EE population can be extended to the larger MITT population. For this reason, from this point on, this report will focus on the MITT population results, unless stated otherwise.

TABLE 6.1
Summary Statistics of Key Testosterone Pharmacokinetic Parameters (Cₘᵢₙ, Cₘₐₓ,
C_{avg}) by PK Day: Day 42/56 Efficacy Evaluable Population

| | | | Final Dose Group^{a} | | | |
|---|---|---|---|---|---|---|
| | | | 2g gel | 3g gel | 4g gel | |
| PK Day | Parameter | Statistic | (40 mg T)^{b} | (60 mg T)^{b} | (80 mg T)^{b} | All |
| Day 1 | C₀ | N | 15 | 36 | 38 | 89 |
| | | Mean ± | 161.1 ± 99.91 | 197.8 ± 77.31 | 173.0 ± 93.48 | 181.0 ± 88.61 |
| | | SD | | | | |
| | | Median | 145.0 | 214.0 | 177.5 | 193.0 |
| | | Range | 25.0-309.0 | 25.0-359.0 | 25.0-356.0 | 25.0-359.0 |
| | Cₘᵢₙ | N | 15 | 36 | 38 | 89 |
| | | Mean ± | 142.8 ± 95.02 | 184.4 ± 75.28 | 152.0 ± 86.97 | 163.5 ± 84.73 |
| | | SD | | | | |
| | | Median | 139.0 | 191.0 | 150.5 | 171.0 |
| | | Range | 25.0-299.0 | 25.0-359.0 | 25.0-317.0 | 25.0-359.0 |
| | Cₘₐₓ | N | 15 | 36 | 38 | 89 |
| | | Mean ± | 1302.0 ± 754.51 | 704.2 ± 567.8 | 457.4 ± 210.34 | 699.6 ± 569.77 |
| | | SD | | 8 | | |
| | | Median | 1203.0 | 537.0 | 382.5 | 501.0 |
| | | Range | 412.0-2611.0 | 131.0-3162.0 | 148.0-1098.0 | 131.0-3162.0 |
| | C_{avg} | N | 15 | 36 | 38 | 89 |
| | | Mean ± | 543.7 ± 230.09 | 380.0 ± 137.8 | 289.6 ± 100.86 | 369.0 ± 168.03 |
| | | SD | | 6 | | |
| | | Median | 522.5 | 355.2 | 276.8 | 336.5 |
| | | Range | 261.9-1144.0 | 67.9-824.4 | 127.0-627.9 | 67.9-1144.0 |
| Day 14 | Cₘᵢₙ | N | 15 | 36 | 38 | 89 |
| | | Mean ± | 453.5 ± 136.76 | 319.3 ± 66.62 | 195.2 ± 49.11 | 288.9 ± 120.59 |
| | | SD | | | | |
| | | Median | 442.0 | 328.5 | 201.0 | 266.0 |
| | | Range | 266.0-705.0 | 156.0-454.0 | 57.0-269.0 | 57.0-705.0 |
| | | N | 15 | 36 | 38 | 89 |
| | Cₘₐₓ | Mean ± | 2667.1 ± 1155.9 | 1420.9 ± 676. | 768.1 ± 368.43 | 1352.2 ± 946.5 |
| | | SD | 6 | 96 | | 2 |
| | | Median | 2336.0 | 1257.0 | 724.5 | 1044.0 |
| | | Range | 1116.0-5580.0 | 542.0-3684.0 | 279.0-1821.0 | 279.0-5580.0 |
| | C_{avg} | N | 15 | 36 | 38 | 89 |
| | | Mean ± | 907.1 ± 275.71 | 577.9 ± 108.8 | 366.2 ± 83.94 | 543.0 ± 237.40 |
| | | SD | | 7 | | |
| | | Median | 836.2 | 563.3 | 363.7 | 510.8 |
| | | Range | 585.4-1582.3 | 406.2-920.1 | 211.7-557.5 | 211.7-1582.3 |
| Day 42/56 | Cₘᵢₙ | N | 15 | 36 | 38 | 89 |
| | | Mean ± | 299.5 ± 126.22 | 331.1 ± 120.8 | 299.2 ± 93.36 | 312.2 ± 111.69 |
| | | SD | | 3 | | |
| | | Median | 242.0 | 298.0 | 288.0 | 290.0 |
| | | Range | 189.0-601.0 | 96.0-662.0 | 124.0-511.0 | 96.0-662.0 |
| | Cₘₐₓ | N | 15 | 36 | 38 | 89 |
| | | Mean ± | 1592.4 ± 666.52 | 1608.2 ± 835. | 1847.6 ± 1191. | 1707.7 ± 979.6 |
| | | SD | | 10 | 43 | 2 |
| | | Median | 1630.0 | 1547.0 | 1528.5 | 1579.0 |
| | | Range | 482.0-2820.0 | 384.0-3917.0 | 223.0-5311.0 | 223.0-5311.0 |
| | C_{avg} | N | 15 | 36 | 38 | 89 |
| | | Mean ± | 614 ± 205.15 | 625.9 ± 216.9 | 640.4 ± 231.32 | 630.2 ± 219.09 |
| | | SD | | 1 | | |
| | | Median | 588.8 | 616.8 | 616.9 | 616.6 |
| | | Range | 317.6-1002.0 | 265.2-1250.7 | 178.7-1416.9 | 178.7-1416.9 |
| Day 182^{c} | Cₘᵢₙ | N | 15 | 32 | 36 | 83 |
| | | Mean ± | 295.9 ± 111.44 | 306.1 ± 116.4 | 299.0 ± 102.06 | 301.2 ± 108.19 |
| | | SD | | 0 | | |
| | | Median | 332.0 | 305.5 | 297.0 | 305.0 |
| | | Range | 117.0-457.0 | 25.0-562.0 | 133.0-498.0 | 25.0-562.0 |
| | Cₘₐₓ | N | 15 | 32 | 36 | 83 |
| | | Mean ± | 1224.7 ± 528.13 | 1352.5 ± 698. | 1387.5 ± 887.4 | 1344.6 ± 756.1 |
| | | SD | | 59 | 2 | 1 |
| | | Median | 1165.0 | 1271.0 | 1107.5 | 1165.0 |
| | | Range | 423.0-2276.0 | 381.0-3098.0 | 310.0-4071.0 | 310.0-4071.0 |
| | C_{avg} | N | 15 | 32 | 36 | 83 |
| | | Mean ± | 548.2 ± 156.93 | 592.5 ± 194.1 | 596.0 ± 211.40 | 586.0 ± 194.54 |
| | | SD | | 3 | | |
| | | Median | 563.3 | 564.7 | 585.8 | 581.0 |
| | | Range | 344.0-879.4 | 243.2-977.9 | 224.5-1087.8 | 224.5-1087.8 |

^{a}Subjects in the 2g and 4g groups received 3g daily only for the first 28 days; subjects in the 3g group received 3g daily for all 182 days.
^{b}For each gram of gel, 20 mg testosterone was applied to the skin.
^{c}Subject 007-118 (4g group) was not included in Day 182 statistics because only one testosterone sample was available for that subject on that day.

TABLE 6.1 (Ctd)
Summary Statistics of Key Testosterone Pharmacokinetic Parameters (Cₘᵢₙ, Cₘₐₓ,
C_{avg}) by PK Day: Day 42/56 Efficacy Evaluable Population

| | | | Final Dose Group^{a} | | | |
|---|---|---|---|---|---|---|
| | | | 2 g gel | 3 g gel | 4 g gel | |
| PK Day | Parameter | Statistic | (40 mg T)^{b} | (60 mg T)^{b} | (80 mg T)^{b} | All |
| Day | Cₘᵢₙ | N | 15 | 32 | 36 | 83 |
| 182^{c} | | Mean ± | 295.9 ± 111.44 | 306.1 ± 116.40 | 299.0 ± 102.06 | 301.2 ± 108.19 |
| | | SD | | | | |
| | | Median | 332.0 | 305.5 | 297.0 | 305.0 |
| | | Range | 117.0-457.0 | 25.0-562.0 | 133.0-498.0 | 25.0-562.0 |
| | Cₘₐₓ | N | 15 | 32 | 36 | 83 |
| | | Mean ± | 1224.7 ± 528.13 | 1352.5 ± 698.59 | 1387.5 ± 887.42 | 1344.6 ± 756.11 |
| | | SD | | | | |
| | | Median | 1165.0 | 1271.0 | 1107.5 | 1165.0 |
| | | Range | 423.0-2276.0 | 381.0-3098.0 | 310.0-4071.0 | 310.0-4071.0 |
| | C_{avg} | N | 15 | 32 | 36 | 83 |
| | | Mean ± | 548.2 ± 156.93 | 592.5 ± 194.13 | 596.0 ± 211.40 | 586.0 ± 194.54 |
| | | SD | | | | |
| | | Median | 563.3 | 564.7 | 585.8 | 581.0 |
| | | Range | 344.0-879.4 | 243.2-977.9 | 224.5-1087.8 | 224.5-1087.8 |

^{a}Subjects in the 2 g and 4 g groups received 3 g daily only for the first 28 days; subjects in the 3 g group received 3 g daily for all 182 days.
^{b}For each gram of gel, 20 mg testosterone was applied to the skin.
^{c}Subject 007-118 (4 g group) was not included in Day 182 statistics because only one testosterone sample was available for that subject on that day.

TABLE 6.2
Summary Statistics of Key Testosterone Pharmacokinetic Parameters (Cₘᵢₙ, Cₘₐₓ, C_{avg}) by PK Day:
Day 42/56 MITT Population

| | | | Final Dose Group^{a} | | | |
|---|---|---|---|---|---|---|
| PK DAY | PARAMETER | STATISTIC | 2 g Gel^{b} | 3 g Gel^{b} | 4 g Gel^{b,c} | ALL DOSES |
| DAY 1 | C0 | N | 29 | 71 | 63 | 163 |
| | | MEAN ± SD | 184.6 ± 106.07 | 209.4 ± 135.23 | 202.4 ± 113.07 | 202.3 ± 121.73 |
| | | MEDIAN | 167.0 | 210.0 | 198.0 | 201.0 |
| | | RANGE | 25.0-372.0 | 25.0-906.0 | 25.0-675.0 | 25.0-906.0 |
| | CMIN | N | 29 | 71 | 63 | 163 |
| | | MEAN ± SD | 164.1 ± 94.75 | 178.5 ± 90.88 | 167.2 ± 84.98 | 171.6 ± 89.01 |
| | | MEDIAN | 151.0 | 184.0 | 179.0 | 178.0 |
| | | RANGE | 25.0-336.0 | 25.0-493.0 | 25.0-335.0 | 25.0-493.0 |
| | CMAX | N | 29 | 71 | 63 | 163 |
| | | MEAN ± SD | 1109.4 ± 688.07 | 634.6 ± 449.16 | 447.9 ± 201.98 | 646.9 ± 488.25 |
| | | MEDIAN | 1035.0 | 530.0 | 388.0 | 502.0 |
| | | RANGE | 168.0-2611.0 | 131.0-3162.0 | 148.0-1098.0 | 131.0-3162.0 |
| | CAVG | N | 29 | 71 | 63 | 163 |
| | | MEAN ± SD | 501.6 ± 206.81 | 362.4 ± 131.81 | 292.7 ± 101.64 | 360.3 ± 155.59 |
| | | MEDIAN | 460.8 | 348.6 | 276.8 | 332.4 |
| | | RANGE | 111.6-1144.0 | 67.9-824.4 | 102.5-627.9 | 67.9-1144.0 |
| DAY 14 | CMIN | N | 29 | 71 | 63 | 163 |
| | | MEAN ± SD | 443.7 ± 121.76 | 294.4 ± 80.87 | 198.0 ± 45.39 | 283.7 ± 116.89 |
| | | MEDIAN | 433.0 | 290.0 | 203.0 | 259.0 |
| | | RANGE | 265.0-705.0 | 25.0-521.0 | 57.0-269.0 | 25.0-705.0 |
| | CMAX | N | 29 | 71 | 63 | 163 |
| | | MEAN ± SD | 2653.3 ± 1166.17 | 1313.3 ± 594.40 | 689.6 ± 331.41 | 1310.6 ± 950.00 |
| | | MEDIAN | 2375.0 | 1127.0 | 629.0 | 999.0 |
| | | RANGE | 1116.0-5739.0 | 328.0-3684.0 | 279.0-1821.0 | 279.0-5739.0 |
| | CAVG | N | 29 | 71 | 63 | 163 |
| | | MEAN ± SD | 885.9 ± 232.66 | 555.3 ± 130.92 | 349.3 ± 74.81 | 534.5 ± 233.54 |
| | | MEDIAN | 854.1 | 535.1 | 336.1 | 473.4 |
| | | RANGE | 515.9-1582.3 | 257.7-968.2 | 211.7-557.5 | 211.7-1582.3 |
| DAY 42/56 | CMIN | N | 29 | 71 | 63 | 163 |
| | | MEAN ± SD | 292.3 ± 140.62 | 326.2 ± 121.15 | 290.8 ± 88.13 | 306.5 ± 114.17 |
| | | MEDIAN | 242.0 | 305.0 | 275.0 | 287.0 |
| | | RANGE | 140.0-700.0 | 68.0-796.0 | 124.0-511.0 | 68.0-796.0 |
| | CMAX | N | 29 | 71 | 63 | 163 |
| | | MEAN ± SD | 1749.1 ± 868.43 | 1604.0 ± 775.35 | 1580.6 ± 1059.08 | 1620.8 ± 907.24 |
| | | MEDIAN | 1653.0 | 1366.0 | 1300.0 | 1375.0 |
| | | RANGE | 364.0-4401.0 | 384.0-3917.0 | 223.0-5311.0 | 223.0-5311.0 |
| | CAVG | N | 29 | 71 | 63 | 163 |
| | | MEAN ± SD | 649.1 ± 226.13 | 637.5 ± 240.04 | 593.4 ± 217.06 | 622.5 ± 228.74 |
| | | MEDIAN | 633.4 | 612.6 | 579.7 | 594.4 |
| | | RANGE | 317.6-1327.8 | 265.2-1714.2 | 178.7-1416.9 | 178.7-1714.2 |
| DAY 182 | CMIN | N | 26 | 61 | 59 | 146 |
| | | MEAN ± SD | 290.6 ± 113.55 | 303.4 ± 123.59 | 296.1 ± 100.67 | 298.2 ± 112.34 |
| | | MEDIAN | 293.5 | 276.0 | 284.0 | 282.0 |
| | | RANGE | 77.0-472.0 | 25.0-790.0 | 126.0-559.0 | 25.0-790.0 |
| | CMAX | N | 26 | 61 | 59 | 146 |
| | | MEAN ± SD | 1297.3 ± 687.95 | 1190.2 ± 682.89 | 1219.0 ± 778.07 | 1220.9 ± 719.84 |
| | | MEDIAN | 1200.5 | 1042.0 | 990.0 | 1040.5 |
| | | RANGE | 423.0-3198.0 | 265.0-3098.0 | 310.0-4071.0 | 265.0-4071.0 |
| | CAVG | N | 26 | 61 | 59 | 146 |
| | | MEAN ± SD | 560.4 ± 178.05 | 551.2 ± 203.76 | 559.0 ± 200.43 | 556.0 ± 196.80 |
| | | MEDIAN | 565.2 | 516.7 | 538.1 | 529.0 |
| | | RANGE | 296.1-879.4 | 206.5-989.4 | 224.5-1087.8 | 206.5-1087.8 |

^{a}Subjects in the 2 g and 4 g groups received 3 g daily only for the first 28 days; subjects in the 3 g group received 3 g daily for all 182 days.
^{b}For each gram of gel, 20 mg testosterone was applied to the skin.
^{c}Subject 007-118 (4 g group) was not included in Day 182 statistics because only one testosterone sample was available for that subject on that day

The analysis below focuses on each subgroup separately. An examination of those subjects whose dose remained unchanged throughout the study (3 g group) is compared and contrasted to the subjects whose dose was adjusted (2 g and 4 g groups).

Subgroup Without Dose Change: 3 g Group

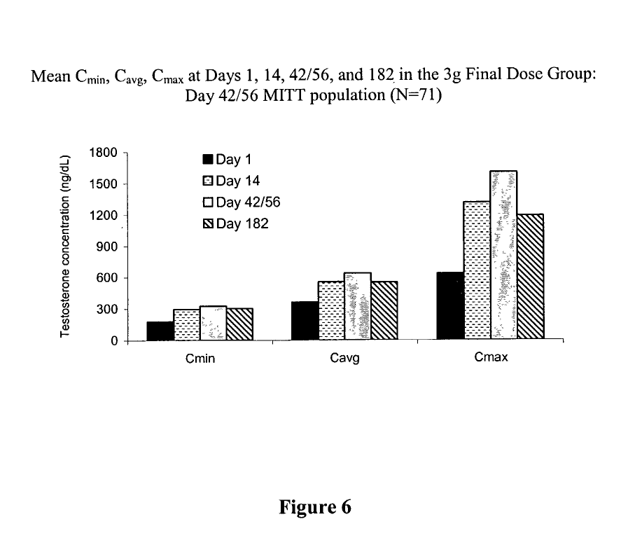

Mean Cₘᵢₙ, C_{avg}, and Cₘₐₓ values for the subgroup of 71 Day 42/56 MITT subjects whose dose was not changed on Day 29 (3 g CP601B, 60 mg testosterone) are shown in FIG. 6 for all PK days.

There was an increase from Day 1 to Day 14 in all the mean PK parameter values in these subjects. From Day 14 on, however, there were only minimal changes (p>0.05) in the mean PK parameters between days, suggesting that the steady-state testosterone concentration was reached by Day 14.

Additionally, since data on Days 14, 42, and 182 can be considered as repeated measurements in the 3 g group, inter- and intra-individual coefficient of variations (CVs) for all three PK parameters were calculated; and results for both the Day 42/56 EE and Day 42/56 MITT subjects of the 3 g group are shown in Table 7.

TABLE 7
Inter- and Intraindividual Coefficient of Variations (% RSD) for Key
Testosterone Pharmacokinetic Parameters (Cₘᵢₙ, Cₘₐₓ, C_{avg}) in the 3 g Final Dose Group

| | | Day 42/56 EE Subjects | | Day 42/56 MITT Subjects | |
|---|---|---|---|---|---|
| | | Inter- | Intra- | Inter- | Intra- |
| | | individual^{a} | individual^{b} | individual^{a} | individual^{b} |
| | N | 36 | | 71 | |
| C_{avg} CV (% RSD) | Mean (SD) | 28.8 (8.64) | 21.8 (11.17) | 32.7 (7.89) | 22.9 (11.95) |
| | Median | 32.8 | 20.7 | 36.8 | 21.0 |
| | Range | 18.8-34.7 | 3.8-51.1 | 23.6-37.7 | 3.8-54.5 |
| Cₘᵢₙ CV (% RSD) | Mean (SD) | 31.8 (9.50) | 22.1 (14.76) | 35.5 (7.41) | 24.4 (17.29) |
| | Median | 36.5 | 19.5 | 37.1 | 19.5 |
| | Range | 20.9-38.0 | 4.2-77.6 | 27.5-42.0 | 4.2-80.3 |
| Cₘₐₓ CV (% RSD) | Mean (SD) | 50.4 (2.40) | 37.0 (17.63) | 50.3 (6.30) | 36.4 (17.86) |
| | Median | 51.7 | 36.7 | 48.3 | 31.7 |
| | Range | 47.6-51.9 | 3.3-80.9 | 45.3-57.4 | 3.3-80.9 |

^{a}Inter-individual coefficient of variation (CV = SD/mean) is calculated over all subjects (36 EE subjects or 71 MITT subjects) for each sampling day. There are 3 sampling days (Days 14, 42, 182) resulting in 3 interindividual CVs for each population. The statistics for the 3 CVs are presented.
^{b}Intra-individual coefficient of variation (CV = SD/mean) is calculated for each subject over Day 14, Day 42, and Day 182, resulting in 71 CVs for the 71 MITT subjects and 36 CVs for the 36 EE subjects. The statistics for these CVs are presented.
KEY: CV: coefficient of variation; RSD: relative standard deviation

The CV for the PK parameters in the Day 42/56 EE and MITT populations were similar. As expected, the intra-individual CV was lower than the interindividual CV. In general, both were reasonably low for a semi-solid transdermal product (without device-controlled delivery) and over a study period of 6 months.

Subgroups with Dose Changes on Day 29: 2 g and 4 g Groups

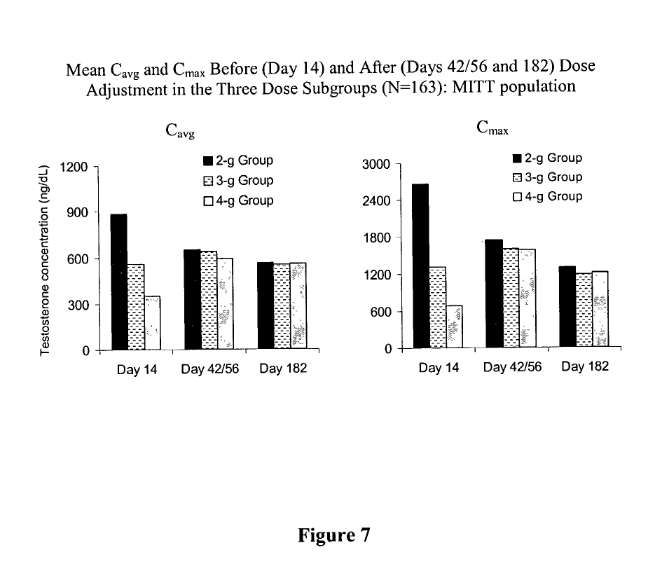

As expected from their high mean testosterone concentration-time profiles, the 29 subjects in the Day 42/56 MITT group whose dose was decreased from 3 g to 2 g CP601B on Day 29 had higher C_{avg} and Cₘₐₓ on Days 1 and 14 than subjects in the other two subgroups (Table 6.2 and FIG. 7). Conversely, mean C_{avg} and Cₘₐₓ in the 4 g group (N=63) were the lowest of the three groups on Day 1 and Day 14.

After subjects in the 2 g and 4 g groups switched from 3 g gel to their final dose, mean C_{avg} and Cₘₐₓ values changed accordingly (up in the 4 g group and down in the 2 g group) and remained fairly constant from Day 42/56 until the end of the study.

The significance of the change in Cₘᵢₙ, C_{avg}, and Cₘₐₓ values before (Day 14) and after (Days 42/56 and 182) dose adjustment in the MITT population was further analyzed by conducting paired comparisons. As shown on Table 8, all three PK parameters were significantly decreased in the 2 g group after dose adjustment (p<0.0001), as would be expected from a decrease in dose. This was true for both comparisons, i.e. Day 14 to Day 42/56 or Day 14 to Day 182. Conversely, in the 4 g group, there was a significant increase in all PK parameters following the increase in dose (p<0.0001). In the subgroup where the dose had not been changed (3 g group) there was a significant change in Cₘᵢₙ, C_{avg}, and Cₘₐₓ between Day 14 and Day 42/56. However, when Day 14 was compared to Day 182, no significant changes in Cₘᵢₙ, C_{avg}, and Cₘₐₓ were observed in neither the MITT nor the EE population. These results provided evidence that there were no overall significant changes in Cₘᵢₙ, C_{avg} and Cₘₐₓ in the 3 g group once steady state had been reached. Therefore, an adjustment in the CP601B dose correlated with expected and highly significant changes in all three key PK parameters. Thus, an increase in dose for those subjects with testosterone concentrations below the PR and a decrease in dose for those with peak concentrations above this range are a successful means of individualizing CP601B doses for testosterone replacement therapy in hypogonadal men.

TABLE 8
Comparison of C_{avg}, Cₘᵢₙ, and Cₘₐₓ Before (Day 14) and
After (Days 42/56 and 182) Dose Adjustment: MITT Population

| | | Final Dose Group^{a} | | |
|---|---|---|---|---|
| | | Mean (SD) | | |
| | | 2 g gel | 3 g gel | 4 g gel |
| | | (40 mg T)^{b} | (60 mg T)^{b} | (80 mg T)^{b} |
| | | N = 26 | N = 61 | N = 59 |
| C_{avg} | Day 14 | 885 (232.7) | 555.3 (130.9) | 349.3 (74.8) |
| | Day 42/56 | 649.1 (226.1) | 637.5 (240.0) | 593.4 (217.1) |
| | Day 182 | 560.4 (178.1) | 551.2 (203.8) | 559.0 (200.4) |
| | p value^{c} | <0.0001 | 0.0004 | <0.0001 |
| | p value^{d} | <0.0001 | 0.9350 | <0.0001 |
| Cₘᵢₙ | Day 14 | 443.7 (121.8) | 294.4 (80.9) | 198.0 (45.4) |
| | Day 42/56 | 292.3 (140.6) | 326.2 (121.2) | 290.8 (88.1) |
| | Day 182 | 290.6 (113.5) | 303.4 (123.6) | 296.1 (100.7) |
| | p value^{c} | <0.0001 | 0.0276 | <0.0001 |
| | p value^{d} | <0.0001 | 0.7907 | <0.0001 |
| Cₘₐₓ | Day 14 | 2653.3 (1166.2) | 1313.3 (594.4) | 689.6 (331.4) |
| | Day 42/56 | 1749.1 (868.4) | 1604.0 (775.4) | 1580.6 (1059.1) |
| | Day 182 | 1297.3 (687.9) | 1190.2 (682.9) | 1219.0 (778.1) |
| | p value^{c} | <0.0001 | 0.0024 | <0.0001 |
| | p value^{d} | <0.0001 | 0.3775 | <0.0001 |

^{a}Subjects in the 2 g and 3 g groups received 3 g daily only for the first 28 days; subjects in the 3 g group received 3 g testosterone daily for all 182 days.
^{b}For each gram of gel, 20 mg testosterone was applied to the skin.
^{c}Wilcoxon signed ranked test for change from Day 14 to Day 42/56
^{d}Wilcoxon signed ranked test for change from Day 14 to Day 182

Comparison Across Subgroups Following Dose Adjustment

On Days 42/56 and 182, the mean values for Cₘᵢₙ, Cₘₐₓ and C_{avg} were similar among Day 42/56 MITT subjects of all three subgroups (FIG. 7; Table 8).

In conclusion, the results of the analysis of Cₘᵢₙ, C_{avg}, and Cₘₐₓ confirmed those obtained from the mean testosterone profiles. There were large differences in the three PK parameters, particularly with C_{avg} and Cₘₐₓ on Day 14, which disappeared following dose adjustment. Dose adjustment was highly successful in decreasing testosterone concentrations and the corresponding PK parameters that were identified as too high, or increasing those that were too low.

Cₘᵢₙ, C_{avg}, Cₘₐₓ: Correlation with BMI and Weight

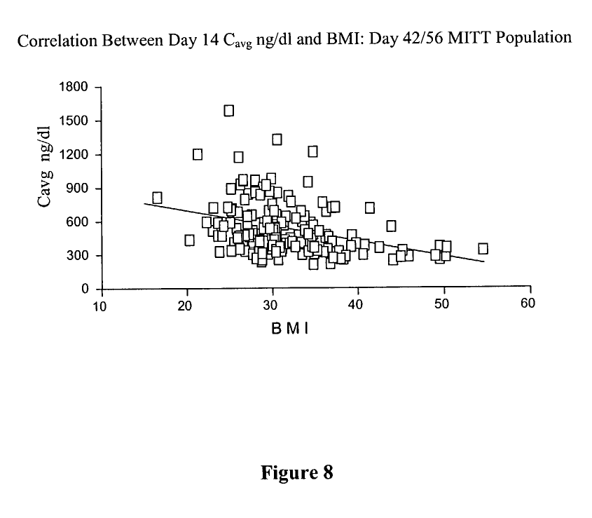

The correlation of Cₘᵢₙ, C_{avg} and Cₘₐₓ with BMI or weight was first studied on Day 14 when all subjects were using the same 3 g dose of gel. There was a highly significant negative correlation between both BMI and weight with C_{avg} and Cₘₐₓ in the MITT population (Table 9 and FIG. 8). For Cₘᵢₙ, the correlation was significant but weaker.

TABLE 9
Correlation Between the Key PK Parameters on
Day 14 and BMI and Weight: Day 42/56 MITT Population

| Variable | Statistics | Cₘᵢₙ | C_{avg} | Cₘₐₓ |
|---|---|---|---|---|
| | N | 163 | 163 | 163 |
| BMI | p value | 0.0160 | <.0001 | <.0001 |
| | R² | 0.0355 | 0.1359 | 0.1395 |
| Weight | p value | 0.0153 | <.0001 | <.0001 |
| | R² | 0.0360 | 0.1263 | 0.1167 |

The analysis was repeated on Day 42/56 after the dose had been adjusted for each subject to compensate for such factors as BMI or weight (Table 10). Correlations were not significant in the 2 g and the 3 g groups, with the exception of Cₘₐₓ in the 3 g group. In contrast, in the 4 g group, all correlations remained significant, particularly for C_{avg} and Cₘₐₓ, with more than 19% of the total variance captured by either of these body measures. Based on mean values for BMI in the 2 g (27.9±4.7 kg/m²), 3 g (31.2±4.4 kg/m²), and 4 g (34.1±7.7 kg/m²) subgroups of the Day 42/56 MITT subjects, the 4 g subgroup had the most obese subjects, suggesting that a further increase in dose may be beneficial for morbidly obese hypogonadal men to achieve optimal testosterone replacement.

TABLE 10
Correlation Between the Key PK Parameters on Day 42/56
and BMI and Weight: Day 42/56 MITT Population

| Dose Group | Variable | Statistics | Cₘᵢₙ | C_{avg} | Cₘₐₓ |
|---|---|---|---|---|---|
| 2 g gel/40 mg T | | N | 29 | 29 | 29 |
| | BMI | p value | 0.3020 | 0.8809 | 0.9780 |
| | | R² | 0.0394 | 0.0008 | 0.0000 |
| | Weight | p value | 0.9502 | 0.5758 | 0.9956 |
| | | R² | 0.0001 | 0.0117 | 0.0000 |
| 3 g gel/60 mg T | | N | 71 | 71 | 71 |
| | BMI | p value | 0.7488 | 0.2873 | 0.0101 |
| | | R² | 0.0015 | 0.0164 | 0.0920 |
| | Weight | p value | 0.4806 | 0.1232 | 0.0217 |
| | | R² | 0.0072 | 0.0341 | 0.0740 |
| 4 g gel/80 mg T | | N | 63 | 63 | 63 |
| | BMI | p value | 0.0090 | <.0001 | <.0001 |
| | | R² | 0.1067 | 0.2575 | 0.2360 |
| | Weight | p value | 0.0417 | 0.0003 | 0.0002 |
| | | R² | 0.0662 | 0.1908 | 0.2051 |

Calculation of The Relative Systemic Bioavailability of CP601B

The calculation of the relative systemic bioavailability of testosterone dosage forms is difficult because of the well-documented feedback inhibition of endogenous testosterone following exogenous administration (Behre H M, Neischlag E. In: Nieschlag E, Behre H M, eds. Testosterone Action—Deficiency—Substitution. 2nd ed. Germany: Springer-Verlag; 1998:329-48). Due to this feedback inhibition, endogenous secretion cannot be assumed constant and, consequently, endogenous testosterone concentrations at treatment steady-state can not be assumed to equal the pretreatment testosterone concentrations. As a result, testosterone absorption from an exogenous source cannot be obtained simply by subtracting the baseline levels from the measured testosterone concentrations at steady state.

By using the subset of study subjects who had no detectable endogenous component (i.e. baseline testosterone concentrations were below 50 ng/dL detection limit on time 0 Day), these issues were circumvented since all measurable serum testosterone could be considered of exogenous origin. This subject population was used to estimate the relative systemic bioavailability of CP601B.

Calculations were performed using C_{avg} on Day 14 when all men in that subset were on the 3 g dose of gel (60 mg T). The relative systemic bioavailability of CP601B was estimated to be 12.2±4.6%

Relevance of CP601B Therapy as a Single Daily Dose Regimen

In this section, the ability of CP601B to raise and maintain testosterone concentrations significantly above pretreatment levels for the entire 24-hour dosing interval is discussed.

Summary statistics on testosterone concentrations at each blood collection time point (Days 1, 14, 42/56 and 182) for Day 42/56 EE, MITT, and ITT subjects (data not shown) support a model of drug input in which there is both rapid transdermal absorption of testosterone in the few hours following application of CP601B, followed by slow continuous release throughout the 24-hour dosing interval. The rapid absorption component is apparent in most subjects from the rise in the testosterone concentration to a peak at 2 to 4 hours. The concentration then falls to a nearly constant value that is sustained from 12 to 24 hours.

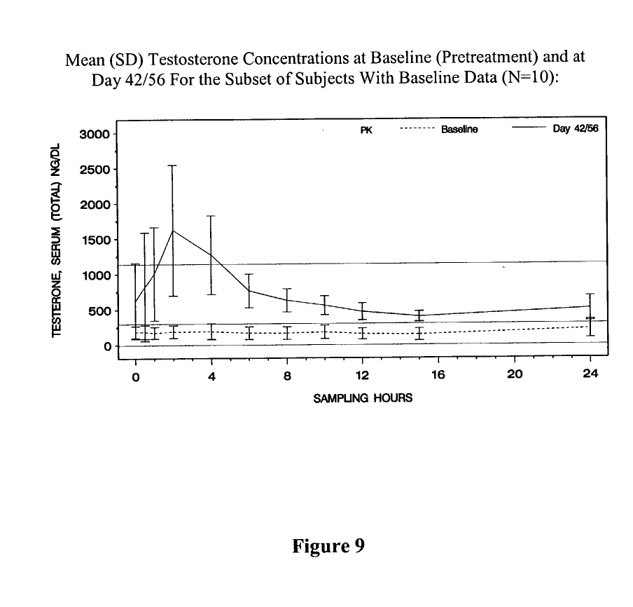

As shown in FIG. 9, in the subset of subjects with 24-hour pretreatment profiles (n=10), the mean testosterone concentration-time profile at Day 42/56 was clearly raised to above the lower limit of the PR (300 ng/dL) compared to the pretreatment profile for the whole 24-hour dosing interval (All 10 subjects were part of the Day 42/56 MITT population; six of the 10 were also in the Day 42/56 EE population.)

Summary statistics for Cₘᵢₙ, C_{avg}, and Cₘₐₓ at pretreatment and at Day 42/56 are shown in Table 11. There was a highly significant increase in all PK parameters (p<0.002) between pretreatment and Day 42/56. Of particular interest was the highly significant mean increase in Cₘᵢₙ (Δ=215.3 ng/dL) that demonstrated that the lowest testosterone concentration of the entire 24-hour profile was significantly higher than the pretreatment concentration. Thus, this analysis clearly provided evidence that steady-state testosterone concentrations in dose-adjusted subjects were significantly higher than pretreatment levels throughout the 24-hour dosing interval.

TABLE 11
Summary Statistics on Cₘᵢₙ, C_{avg}, and Cₘₐₓ (ng/dL) at Baseline and Day 42/56 in
the Subset of Subjects with Both Baseline and Day 42/56 Data (N = 10)

| | | | | Difference (Δ) | |
|---|---|---|---|---|---|
| | | Baseline | Day 42/56 | Day 42/56 - Baseline | p value^{a} |
| Cₘᵢₙ | Mean (SD) | 129.4 (69.02) | 344.7 (79.66) | 215.3 (91.10) | 0.0020 |
| | Median | 148.5 | 341.0 | 258.5 | |
| | Range | 25.0-212.0 | 215.0-480.0 | 64.0-296.0 | |
| C_{avg} | Mean (SD) | 175.8 (91.82) | 670.2 (179.12) | 494.4 (170.01) | 0.0020 |
| | Median | 225.1 | 702.9 | 540.4 | |
| | Range | 27.6-254.5 | 361.8-906.6 | 178.1-658.2 | |
| Cₘₐₓ | Mean (SD) | 239.6 (118.21) | 1846.3 (913.56) | 1606.7 (872.60) | 0.0020 |
| | Median | 282.5 | 1863.5 | 1568.5 | |
| | Range | 50.0-398.0 | 598.0-3147.0 | 350.0-2821.0 | |

^{a}Wilcoxon signed ranked test

Since the analysis above was based on a limited subset of subjects (n=10), a modified approach was used to test the same hypothesis in the entire MITT population (n=163). In this modified approach, the time-weighted average testosterone concentration in the last 12 hours of the Day 42/56 dosing interval was calculated (C_{avg12-24}, defined by AUC₁₂₋₂₄/12) and compared to testosterone concentrations on entry (Day 1 C₀) in a pairwise manner. The results of this analysis are shown in Table 12 for the Day 42/56 MITT population.

TABLE 12
Comparison Between Day 42/56 C_{avg 12-24} and
Day 1 C₀: Day 42/56 MITT Population

| | Final Dose Group^{a} | | | |
|---|---|---|---|---|
| | 2 g gel | 3 g gel | 4 g gel | |
| | (40 mg T)^{b} | (60 mg T)^{b} | (80 mg T)^{b} | All |
| | N = 29 | N = 71 | N = 63 | N = 163 |
| Day 1 C₀^{c} | | | | |
| Mean (SD) | 185 (106.1) | 209 (135.2) | 202 (113.1) | 202 (121.7) |
| Median | 167 | 210 | 198 | 201 |
| Range | 25-372 | 25-906 | 25-675 | 25-906 |

Day 42/56 C_{avg 12-24}^{d}

| | | | | |
|---|---|---|---|---|
| Mean (SD) | 454 (163.9) | 467 (220.6) | 413 (151.3) | 444 (187.2) |
| Median | 399 | 411 | 369 | 388 |
| Range | 240-861 | 189-1280 | 170-967 | 170-1280 |

Difference between Day 42/56 C_{avg 12-24} and Day 1, hr 0

| | | | | |
|---|---|---|---|---|
| Mean (SD) | 270 (219.5) | 257 (263.0) | 211 (184.7) | 242 (227.8) |
| Median | 279 | 207 | 178 | 207 |
| Range | −62-766 | −416-1100 | −245-659 | −416-1100 |
| p value^{c} | <0.0001 | <0.0001 | <0.0001 | <0.0001 |

^{a}Subjects in the 2 g and 3 g groups received 3 g daily only for the first 28 days; subjects in the 3 g group received 3 g daily for all 182 days.
^{b}For each gram of gel, 20 mg testosterone was applied to the skin.
^{c}Day 1 C₀ is the testosterone concentration in the first blood sample (Hr 0) on Day 1 before the first dose of CP601B is applied.
^{d}Day 42/56 C_{avg 12-24} is the time-weighted average (AUC₁₂₋₂₄ divided by 12) of the testosterone concentrations between 12 and 24-hours postdose on Day 42/56.
^{e}Wilcoxon signed ranked test

Based on the analyses of results in all dose groups, there was a highly significant difference between testosterone concentrations in the second 12 hours of the steady-state profile and the pretreatment testosterone concentration. Because the last 12 hours of the profile are those likely to provide the lowest testosterone concentrations, these results support the conclusion that testosterone concentrations are maintained above baseline levels for the full 24-hour dosing interval.

In summary, at steady state in properly dose-adjusted hypogonadal subjects, a single daily dose of CP601B provides mean testosterone concentrations that are sustained above baseline levels for the full 24-hour dosing interval.

Efficacy Endpoints: Maintenance of Testosterone Concentration Within the Physiologic Range

The primary objective of this study was to determine the effectiveness of CP601B in maintaining testosterone concentrations of hypogonadal men within the PR of healthy young men. The physiologic range for males, 300-1140 ng/dL, was used as a target for successful therapy.

The primary efficacy endpoint was defined as the proportion of subjects with both C_{avg} and Cₘᵢₙ within the PR on Day 42/56, the primary efficacy day. The primary trial endpoint was met on the primary efficacy day if the lower bound of the 95% CI for the proportion of subjects with both C_{avg} and Cₘᵢₙ within the PR on Day 42/56 was higher than the non-inferiority margin of 20% (for a historical point estimate of 35% with an allowable delta of 15%).

The requirement of Cₘᵢₙ being within the PR for the entire dosing interval as a component of the primary endpoint was quite strict and not necessarily clinically required for successful therapy. Therefore, other endpoints were also included in the analyses. These included: 1) the proportion of subjects with C_{avg} within the PR, 2) the proportion of subjects with both C_{avg} within the PR and testosterone concentrations above the lower end of the PR for at least 80% of the dosing interval, and 3) the proportion of subjects with testosterone concentrations within the PR for at least 80% of the dosing interval. Because full PK profiles were also available on Day 182, success rates for the various endpoints at Day 42/56 were also calculated for Day 182.

Results for the primary endpoint and the others are discussed below. Statistical analyses of the efficacy endpoints for the Day 42/56 EE, MITT, and ITT populations are summarized in Table 13;the same analyses for Day 182 are summarized in Table 14.

TABLE 13
Statistical Analysis of C_{avg} and Cₘᵢₙ for Serum Testosterone on Pharmacokinetic Profile Day 42/56:
Day 42/56 EE, MITT, and ITT Populations

| | DAY 42/56 | | | | | |
|---|---|---|---|---|---|---|
| | EFFICACY | | DAY 42/56 | | | |
| | EVALUABLE POPULATION | | MITT POPULATION | | ITT POPULATION^{a} | |
| | (N = 89) | | (N = 163) | | (N = 201) | |
| | | 95% CONF. | | 95% CONF. | | 95% CONF. |
| CRITERION | N (%) | INT^{b}. | N (%) | INT^{b}. | N (%) | INT^{b} |
| SUBJECTS WITH CAVG IN | 38 (42.7) | (32.4, 53.0) | 68 (41.7) | (34.1, 49.3) | 68 (33.8) | (27.3, 40.4) |
| [300 NG/DL, 1140 NG/DL] AND CMIN >= 300 | | | | | | |
| SUBJECTS WITH CAVG IN | 82 (92.1) | (86.5, 97.7) | 150 (92.0) | (87.9, 96.2) | 150 (74.6) | (68.6, 80.6) |
| [300 NG/DL, 1140 NG/DL] | | | | | | |
| SUBJECTS WITH CAVG IN | 63 (70.8) | (61.3, 80.2) | 116 (71.2) | (64.2, 78.1) | 116 (57.7) | (50.9, 64.5) |
| [300 NG/DL, 1140 NG/DL] AND | | | | | | |
| CONCENTRATION >= 300 NG/DL FOR >= | | | | | | |
| 80% OF DOSING INTERVAL | | | | | | |
| SUBJECTS WITH CONCENTRATION IN | 53 (59.6) | (49.4, 69.7) | 94 (57.7) | (50.1, 65.3) | 94 (46.8) | (39.9, 53.7) |
| [300 NG/DL, 1140 NG/DL] FOR >= 80% OF | | | | | | |
| DOSING INTERVAL | | | | | | |

^{a}Subjects who did not have a PK profile on day 42/56 were included as a failure (i.e., included in the denominator) in the analysis of the ITT population.
^{b}Two-sided 95% confidence interval (p +− z.05 * se (p) computed on the percentage of subjects meeting the specific criteria, using the normal approximation to the binomial.

Primary Efficacy Endpoint

Of the 89 Day 42/56 EE subjects, 38 (42.7%) had testosterone C_{avg} and Cₘᵢₙ within the PR of 300-1140 ng/dL on Day 42/56. The lower bound of the 95% CI (32.4%) was higher than the non-inferiority margin of 20% (for a historical point estimate of 35% with an allowable delta of 15%). Thus, the primary endpoint of the trial was met on the primary efficacy day in the EE population.

Similar results were obtained when the analysis was based on the Day 42/56 MITT population (N=163). Sixty-eight of the 163 Day 42/56 MITT subjects (41.7%) had testosterone C_{avg} and Cₘᵢₙ within the PR. The 95% CI was 34.1%-49.3%; again, the lower bound of the 95% CI was higher than the non-inferiority margin of 20%. Thus, the primary endpoint of the trial was also met on the primary efficacy day in the MITT population.

For the ITT population (N=201), 33.8% (68/201) of subjects had testosterone C_{avg} and Cₘᵢₙ within the PR. The 95% CI was 27.3%-40.4%, still well above the non-inferiority margin of 20%. Therefore, on the primary efficacy day, the primary endpoint of the trial was also met in the ITT population.

In summary, the lower bound of the 95% CI for the percentage of subjects with both Cₘᵢₙ and C_{avg} within the PR was above the 20% non-inferiority margin in all three populations. Therefore, the primary endpoint of the study was met in all three populations (Day 42/56 EE, Day 42/56 MITT, and ITT) on the primary efficacy day.

Secondary Efficacy Endpoints

C_{avg} Within Physiologic Range on Day 42/56

Eighty-two of the Day 42/56 EE subjects (92.1%) had C_{avg} values within the PR on Day 42/56, with a 95% CI of 86.5%-97.7%. The lower bound of the 95% CI was well above the 65% non-inferiority margin (for a historical point estimate of 80% with an allowable delta of 15%).

In the Day 42/56 MITT population, 150 of the 163 subjects (92%) had C_{avg} within the PR (95% CI: 87.9%-96.2%). The lower bound of the 95% CI was also well above the non-inferiority margin selected for comparison for the EE population.

In the ITT population, results were similar: 75% of the subjects had C_{avg} values within the PR, with the lower bound of the 95% CI (68.6%) above the non-inferiority margin.

In summary, 1) 92% of the Day 42/56 EE (n=89), 92% of the Day 42/56 MITT (n=163), and 75% of the ITT subjects (n=201) had C_{avg} values within the PR on Day 42/56, the designated primary efficacy day, and 2) the lower bound of the 95% CI for this percentage was above the 65% non-inferiority margin selected for significance in all three populations.

Proportion of Subjects with Both C_{avg} and Cₘᵢₙ Within the PR on Day 182

As shown on Table 14, 41 of the 84 Day 182 EE subjects (48.8%) had both C_{avg} and Cₘᵢₙ in the PR on Day 182. The lower bound of the 95% CI (38.1%) was well above the non-inferiority margin of 20% (for a historical point estimate of 35% with an allowable delta of 15%).

In the Day 182 MITT population (N=146), results were similar to those obtained on Day 42/56, with 62 subjects (42.5%) with both C_{avg} and Cₘᵢₙ within the PR and a 95% CI of 34.4%-50.5%. Thus, the lower bound of the 95% CI was again well above the non-inferiority margin selected for significance.

In the ITT population (N=201), results were also similar to Day 42/56, with 30.8% of subjects with both Cₘᵢₙ and C_{avg} within the PR on Day 182. As in the other two populations, the lower bound of the 95% CI (24.5%) was higher than the non-inferiority margin (20%).

In summary, results on Day 182 matched those obtained on Day 42/56 for the same endpoint. As observed on Day 42/56, the lower bound of the 95% CI for the percentage of subjects with both Cₘᵢₙ and C_{avg} within the PR on Day 182 was above the 20% non-inferiority margin in all 3 populations studied (Day 182 EE, Day 182 MITT, and ITT). The primary endpoint of the study was thus met in all populations on both the primary and secondary efficacy days.

TABLE 14
Statistical Analysis of C_{avg} and Cₘᵢₙ for Serum Testosterone (ng/dL) on Pharmacokinetic Profile Day 182:
Day 182 EE, MITT, and ITT Populations

| | Day 182 EE | | Day 182 MITT | | | |
|---|---|---|---|---|---|---|
| | Population | | Population | | ITT Population^{a} | |
| | (N = 84) | | (N = 146) | | (N = 201) | |
| | | 95% | | 95% | | 95% |
| | | Conf. | | Conf. | | Conf. |
| Criterion | N (%) | Int.^{b} | N (%) | Int.^{b} | N (%) | Int.^{b} |
| Subjects With C_{avg} in [300 ng/dL, 1140 ng/dL] and | 41 (48.8) | (38.1, | 62 (42.5) | (34.4, | 62 (30.8) | (24.5, |
| Cₘᵢₙ ≧ 300 ng/dL | | 59.5) | | 50.5) | | 37.2) |
| Subjects With C_{avg} in [300 ng/dL, 1140 ng/dL] | 81 (96.4) | (92.5, | 139 (95.2) | (91.7, | 139 (69.2) | (62.8, |
| | | 100.4) | | 98.7) | | 75.5) |
| Subjects With C_{avg} in [300 ng/dL, 1140 ng/dL] and | 63 (75.0) | (65.7, | 105 (71.9) | (64.6, | 105 (52.2) | (45.3, |
| Concentration ≧ 300 ng/dL | | 84.3) | | 79.2) | | 59.1) |
| for 80% of Dosing Interval | | | | | | |
| Subjects With Concentration in | 53 (63.1) | (52.8, | 90 (61.6) | (53.8, | 92 (45.8) | (38.9, |
| [300 ng/dL, 1140 ng/dL] for ≧ 80% of | | 73.4) | | 69.5) | | 52.7) |
| Dosing Interval | | | | | | |

^{a}Subjects who did not have a PK profile on Day 182 were included as failures (i.e., included in the denominator) in the analysis of the ITT population.
^{b}Two-sided 95% confidence interval (p +− z.05 * se (p)) computed on the percentage of subjects meeting the specific criterion, using the normal approximation to the binomial.

Proportion of Subjects with C_{avg} Within the PR on Day 182

Eighty-one of the 84 Day 182 EE subjects (96.4%) and 139 of the 146 Day 182 MITT subjects (95.2%) had C_{avg} within the PR on Day 182. The lower bound of the 95% CI in these two populations (92.5% and 91.7% in the Day 182 EE and MITT populations, respectively) are well above the 65% non-inferiority margin selected for comparison.

In the ITT population, 69.2% of subjects had C_{avg} within the PR and the lower bound of the 95% CI was 62.8%, slightly below the 65% non-inferiority margin.

In summary, 1) over 96% of the Day 182 EE, 95% of the Day 182 MITT, and 69% of the ITT subjects had C_{avg} values within the PR on Day 182, and 2) the lower bound of the 95% CI was well above the 65% non-inferiority margin selected for significance in both the Day 182 EE and MITT populations. It was slightly below in the ITT population.

Other Efficacy Analyses

Five additional efficacy analyses were conducted to further characterize CP601B testosterone concentration-time profiles.

C_{avg} Within the PR and Concentrations ≧300 ng/dL for at Least 80% of Dosing Interval

The efficacy of testosterone replacement therapy is related to the dose applied and the duration of treatment. The primary endpoint was modified to include those subjects who had Cₘᵢₙ values below 300 ng/dL for less than 20% of the dosing interval.

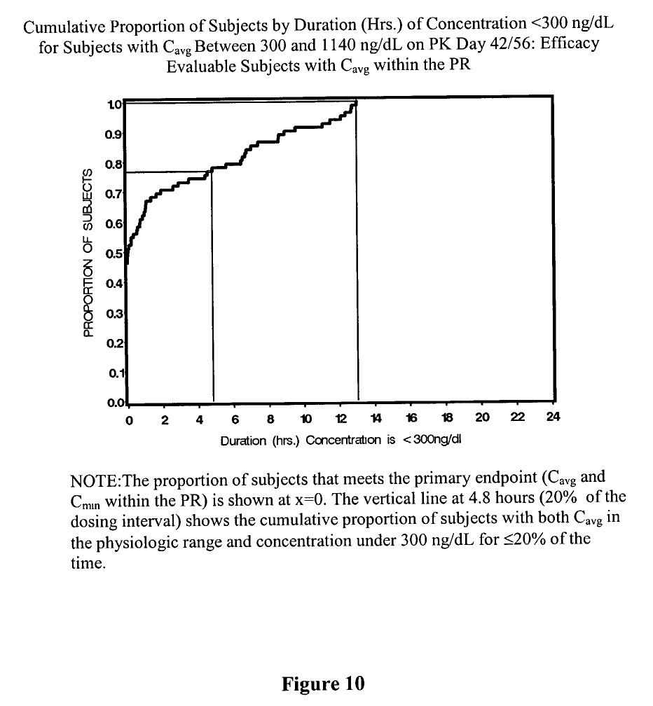

This endpoint was evaluated in Day 42/56 EE subjects with C_{avg} within the PR (FIG. 10). Subjects meeting the primary endpoint had Cₘᵢₙ values above 300 ng/dL, i.e., their duration of testosterone concentrations below 300 ng/dL is 0 hour. Thus, by design, X=0 in FIG. 10 represents the proportion of subjects meeting the primary endpoint (42.7%, Table 13).

Similarly, the proportion of subjects meeting the expanded C_{avg} endpoint discussed in this section is found graphically by the vertical line at X=4.8 hours (20% of the dosing interval) and is shown to be >70%. Of the Day 42/56 EE subjects with Cavg between 300 and 1140 ng/dL, 63 (78%) had both C_{avg} values within the PR and testosterone concentrations ≧300 ng/dL for at least 80% of the time on Day 42/56. In the Day 42/56 MITT population, the proportion of subjects meeting the same endpoint was similar (71.2%). It was lower in the ITT group (57.7%).

On Day 182 (Table 14), 75.0% of the Day 182 EE, 71.9% of the Day 182 MITT and 52.2% of the ITT subjects had both C_{avg} values within the PR and testosterone concentrations ≧300 ng/dL for at least 80% of the time.

Testosterone Concentration within the PR for >80% of the Dosing Interval

Another endpoint of interest to evaluate the effectiveness of CP601B was the proportion of subjects who were within the PR for more than 80% of the dosing interval regardless of whether they were above or below for the remaining 20%. Fifty-three subjects of Day 42/56 EE population (59.6%) had testosterone concentrations within the PR for at least 80% of the time on Day 42/56 (Table 13). The proportion was similar in the MITT group (57.7%), but lower in the ITT group (46.8%).

On Day 182, 63.1% of the Day 182 EE, 61.6% ofthe Day 182 MITT and 45.8% of the ITT subjects had concentrations within the PR for more than 80% of the time (Table 14).

Duration of Testosterone Concentrations Outside the Physiologic Range: Effect of Dose Adjustment

The positive effect of dose adjustment on the key PK parameters Cₘᵢₙ, C_{avg}, and Cₘₐₓ was discussed previously. In this section, the effect of dose adjustment on the duration of testosterone concentration within the PR is described.

Effect of Increasing CP601B Dose: 4 g Group

Subjects in the 4 g group were instructed to start applying a higher dose of gel on Day 28 (from 3 g to 4 g) because their testosterone concentrations were low on Day 14. The positive effect of increasing the gel dose in the 4 g group on the maintenance of testosterone concentrations within the PR is illustrated below.

On Day 14 (before dose adjustment), 74% of the 4 g group subjects had testosterone concentrations under 300 ng/dL for more than 4.8 hours. Following dose adjustment, that number decreased to approximately 25% on Day 56 and 26% on Day 182. On the other hand, increasing the dose slightly increased the number of subjects for which testosterone concentrations were above 1140 ng/dL for more than 4.8 hours, from 0% before dose adjustment to approximately 10% and 11% on Days 56 and 182, respectively. Overall, however, the effect of increasing the dose in that group was positive: The number of subjects outside the PR for more than 4.8 hours went from approximately 74% before adjustment to 40% and 36% on Days 56 and 182, respectively.

Effect of Decreasing CP601B Dose: 2 g Group

Subjects in the 2 g group were instructed to decrease their gel dose from 3 g to 2 g on Day 28 because their Cₘₐₓ on Day 14 were high. On Day 14 (before dose adjustment), 34% of the 2 g group subjects had testosterone concentrations above 1140 ng/dL for more than 4.8 hours. The dose reduction successfully decreased that number to 7% on Day 56 and 0% on Day 182. Overall, the number of subjects outside the PR for more than 4.8 hours increased from 34% on Day 14 to 53% on Day 56, mostly due to an increase in the number of subjects with lower concentrations. There was no change, however, in the total number of subjects outside the PR for more than 4.8 hours between Day 14 and Day 182 (34%). Thus, a decrease in dose resulted in a decrease of the amount of time subjects were supraphysiological, yet had minimal effects on the time within the PR; i.e., the PK profile in the 2 g group was improved following dose adjustment.

3 g Group

In the 3 g group, no dose change occurred; thus, results obtained on Day 14, 42, and 182 were expected to be fairly similar. The number of subjects with testosterone concentrations below 300 ng/dL for less than 4.8 hours remained fairly constant at all three PK days [approximately 83%, 78%, and 81% for Days 14, 42, and 182, respectively]. Similar results were obtained for the number of subjects with testosterone concentrations above 1140 ng/dL for less than 4.8 hours, with approximately 94%, 91%, and 91% for Days 14, 42, and 182, respectively. Overall, there was a slight decrease in the percent of subjects from 78% on Day 14 to 66% on Day 42 and 63% on Day 182 with testosterone concentrations outside the PR for less than 4.8 hours.

Other Serum Hormone Concentrations and Ratios

DHT Concentrations and Serum DHT to Testosterone Ratio

DHT Physiologic Range: 30-85 ng/dL (Esoterix)

Serum DHT concentrations increased from a mean of 18.5±10.8 ng/dL at baseline to a pre-dose level of 78.0±52.0 ng/dL on Day 14, 86.2±50.4 ng/dL on Day 42/56 and 86.4±59.6 ng/dL on Day 182 in Day 42/56 MITT subjects. With the exception of Day 1, the serum DHT concentration after CP601B application was increased transiently above these levels on Days 14, 42/56 and 182; DHT returned to pre-dose levels by the end of 24-hour dosing interval. Similar patterns were observed in the Day 42/56 EE and ITT population. The DHT to testosterone ratio increased from 0.12±0.16 at baseline to 0.21±0.09 on Day 14, 0.22±0.087 on Day 42/56, and 0.22±0.10 on Day 182 in the Day 42/56 MITT group. DHT to testosterone ratios were numerically higher on Day 182 than at baseline, but these differences did not achieve statistical significance. Similar results were observed in the Day 42/56 EE and ITT population.

Serum DHT concentrations and DHT to testosterone ratios during application of CP601B testosterone gel are similar to those reported earlier with AndroGel (Swerdloff RS, Wang C, Cunningham G, et al.. J Clin Endocrinol Metab. 2000; 85(12):4500-4510). and lower than those reported with the scrotal patch (Ahmed S R, Boucher A E, Manni A, Santen R J, Bartholomew M, Demers L M. J Clin Endocrinol Metab. 1988 March;66(3):546-51). Slightly higher DHT concentrations in hypogonadal men treated with the testosterone gels than among those treated with injectable esters are likely due to the conversion of testosterone to DHT in the skin. Comparison of the serum DHT levels in hypogonadal subjects treated with the scrotal patch with those in normal men who had similar testosterone concentrations revealed that subjects treated with the scrotal patch had significantly higher mean serum DHT concentration [315±69 vs. 87±6 ng/dL (10.8±2.4 vs. 2.9±0.2 nmol/L); p<0.001] as well as mean DHT to testosterone ratio [0.6 (range, 0.25-1.1) vs. 0.16 (range, 0.09-0.24); p<0.001] (Ahmed S R, Boucher A E, Manni A, Santen R J, Bartholomew M, Demers L M. J Clin Endocrinol Metab. 1988 March; 66(3):546-51.). The high serum DHT levels in hypogonadal men treated with the scrotal patch were presumably due to increased metabolism of testosterone to DHT by the 5 alpha-reductase in the scrotal skin. There was initial concern after introduction of the scrotal patch that the relatively higher DHT concentrations in hypogonadal men treated with this formulation might adversely affect the prostate. However, long-term surveillance of hypogonadal men treated with the scrotal patch from three to 10 years has not revealed a higher frequency of adverse effects including prostatic disorders or plasma lipid abnormalities among these subjects than among controls (Behre H M, von Eckardstein S, Kliesch S, Nieschlag E. Clin Endocrinol (Oxf) 1999; 50:629-35; Snyder P J, Peachey H, Berlin J A, et al. J Clin Endocrinol Metab. 2000; 85(8):2670-2677). In fact, serum PSA levels and prostate volumes in hypogonadal men treated with the scrotal patch for up to 10 years were not significantly differently from age-matched controls (Behre H M, von Eckardstein S, Kliesch S, Nieschlag E. Clin Endocrinol (Oxf) 1999; 50:629-35.). In two recent, placebo-controlled studies (Kunelius P, Lukkarinen O, Hannuksela M L, Itkonen O, Tapanainen J S. J Clin Endocrinol Metab. 2002 April; 87(4): 1467-72;Ly L P, Jiminez M, Zhuang T N, Celermajer D S, Conway A J, Handelsman D J. J Clin Endocrinol Metab. September 2001; 86(9):4078-4088) of transdermal DHT administration in older men, no significant differences in serum PSA levels or prostate weights between the two groups was observed.

In summary, serum DHT concentrations and DHT to testosterone ratios in healthy hypogonadal men treated with the CP601B testosterone gel are similar to those reported earlier with AndroGel (AndroGel PCT patent application PCT WO 02/17926) and lower than those observed with the scrotal testosterone patch that has demonstrated to be quite safe in long-term follow up studies.

Bioactive Testosterone (BAT) Levels

BAT Physiologic Range: 120-430 ng/dL (Esoterix)

Serum BAT concentrations increased from a mean of 96.8±69.3 ng/dL at baseline to pre-dose levels of 231.7±287.4 ng/dL on Day 14, 226.5±174.9 ng/dL on Day 42/56 and 220.8±157.2 ng/dL on Day 182 in Day 42/56 MITT subjects; all values were within the normal range for adult males (120-430 ng/dL). The serum BAT concentration was transiently increased on Days 1, 14, 42/56 and 182 after CP601B application; with the exception of Day 1, BAT returned to pre-dose levels by the end of 24-hour dosing interval. Similar patterns were observed in the Day 42/56 EE and ITT populations.

Estradiol and E₂/T Ratio

E₂ Physiologic Range: 0.8-3.5 ng/dL (Esoterix)

Testosterone serves as a prohormone; it is converted in the body to two important metabolites: to estradiol 17 beta through the action of aromatase and to DHT through the action of steroid 5-alpha-reductases. While some androgen actions are mediated through binding of testosterone to androgen receptors, a number of important biologic actions of testosterone, especially effects on bone, cognitive function, sexual differentiation of the brain, gonadotropin suppression, plasma lipids and atherosclerosis progression, are mediated through its conversion to estradiol. Testosterone replacement in androgen-deficient men not only increases serum testosterone concentrations into the physiologic range, but also produces desirable increments in serum estradiol concentrations into the physiologic range, in proportion to the increment in serum testosterone concentrations.

At baseline the mean serum estradiol concentrations were within the physiologic male range, although a few androgen-deficient men had serum estradiol levels above the upper limit of the normal range. In the Day 42/56 MITT population, treatment with CP601B was associated with a significant increase in serum estradiol concentrations from a mean of 1.6±0.8 ng/dL at time 0 on Day 1 (baseline) to 3.2±1.8 ng/dL on Day 14, 3.6±1.9 ng/dL on Day 42/56, and 3.4±1.9 on Day 182, well within the range for healthy young men. An evaluation of the 24-hour profile of hormone concentrations indicates that serum estradiol concentrations were numerically slightly higher 2-6 hours after application of CP601B than at time 0 on the various treatment days. Serum estradiol levels during treatment, however, were within the physiological male range in a vast majority of hypogonadal men treated with CP601B. In a small number of hypogonadal men, serum estradiol concentrations increased above the ULN male range (3.5 ng/dL).

The estradiol-to-testosterone ratios were within the physiologic male range and did not significantly change during treatment in the Day 42/56 EE, MITT, or ITT populations, indicating that the increments in serum estradiol levels were in proportion to the increments in serum testosterone concentrations. This is consistent with the low frequency of gynecomastia observed. Of the 201 subjects evaluated for safety, three developed gynecomastia and one additional subject reported breast tenderness. These incidence rates of gynecomastia (1.5%) and breast tenderness (<1%) are in general agreement with previous experience with other testosterone formulations. These data are similar to the published experience with previously approved AndroGel. (Wang C, Swerdoff R S, Iranmanesh A, et al. J Clin Endocrinol Metab 2000; 85:2839-53; Swerdloff R S, Wang C, Cunningham G, Dobs A, Iranmanesh A, Matsumoto A M, Snyder P J, Weber T, Longstreth J, Bennan N.. J Clin Endocrinol Metab. 2000 December; 85(12):4500-10.)

In men, estradiol is derived from aromatization of testosterone in peripheral tissues, largely in the adipose tissue (Braunstein G D. Endocr Relat Cancer. 1999 June; 6(2):315-24; Braunstein G D. N Engl J Med. Feb. 18, 1993; 328(7):490-5). Both systemically delivered or locally produced elevations in estradiol concentrations will promote the growth of hormone-responsive tissues. Alterations in testosterone to estradiol ratios, whether they occur during physiological transitions such as puberty or the neonatal period or as a result of testosterone administration, can be associated with breast enlargement in some individuals. Therefore it is not surprising that three of the four men who developed gynecomastia or breast tenderness were obese with BMIs in excess of 32 kg/m², and had at least the peak serum concentrations of estradiol above the upper limit of physiologic male range. It is likely that increased aromatization of testosterone to estradiol in the adipose tissue in these individuals might have contributed to the development of breast enlargement.

FSH and LH Levels

FSH Physiologic Range: 2.0-9.2 mIU/mL (Esoterix)

LH Physiologic Range: 1.5-9.0 mIU/mL (Esoterix)

Subjects with primary hypogonadism have a defective gonadal function that resulted in high levels of FSH and LH; testosterone administration should lead to reduction of FSH and LH levels in these subjects. For subjects with secondary hypogonadism, the levels of FSH and LH are typically within the normal range. Results reported for an AndroGel study showed that there was a decrease in FSH level in subjects with primary, secondary, and age-related hypogonadism using testosterone gel, especially in subjects requiring the administration of 10 g AndroGel (Swerdloff R S, Wang C, Cunningham G, et al.. J Clin Endocrinol Metab. 2000; 85(12):4500-4510). No significant change in FSH was observed in subjects treated with testosterone patches Swerdoff, ibid.) Both testosterone gel and patch were effective in decreasing LH levels in subjects regardless of their respective types of hypogonadism in the AndroGel study. Serum FSH levels in the Day 42/56 MITT subjects were 10.20±15.39 mIU/mL at baseline, 5.10±11.00 mIU/mL on Day 14, 3.20±8.94 mIU/mL on Day 42/56, and 2.70±5.95 mIU/mL on Day 182. Similar reductions in the FSH levels were also observed in the Day 42/56 EE and ITT population. Serum LH levels in Day 42/56 MITT subjects were 6.10±8.04 mIU/mL at baseline, 3.10±6.73 mIU/mL on Day 14, 1.90±5.35 mIU/mL on Day 42/56, and 1.70±4.15 mIU/mL on Day 182. Similar reductions in the LH levels were also observed in the Day 42/56 EE and ITT population. As anticipated, testosterone replacement therapy with CP601B effectively decreased levels of serum FSH and LH.

SHBG

SHBG Physiologic Range: 24-78 nmol/L (Esoterix)

Mean values for SHBG did not change significantly from baseline at any of the PK evaluation times and days, and were within the normal physiological range. Results were similar for the Day 42/56 EE, MITT and ITT populations

Summary

The pattern of change in BAT, DHT, and estradiol in the Day 42/56 EE, MITT and ITT subjects was similar to that observed with testosterone replacement treatment using other testosterone products. The levels increased from baseline to Day 14, showed a further increase on Day 42/56, and then either decreased slightly or remained constant on Day 182. The value on Day 182 was higher than the baseline value.

The ratio of DHT to testosterone increased from baseline during active treatment, but only by a small percent. The DHT to T ratios were numerically higher on Day 182 than at baseline, but these differences did not achieve statistical significance.

There was no change over time in the ratio of estradiol to testosterone or SHBG levels. As expected, values for FSH and LH decreased over the course of the study.

Effect of 6-month CP601B Treatment on Bone Mineral Density (BMD)

Forty-nine subjects, including 39 Day 42/56 MITT subjects, who had never used testosterone replacement products before entering this study, were considered for BMD evaluations. The mean changes in BMD occurred in the Day 42/56 MITT subjects who had data both at baseline and on Day 182 are summarized in Table 15. Statistically significant increases in BMD occurred at both the hip (Mean: 0.020 gHa/cm2; signed rank p-value of 0.0161) and the lumbar spine (Mean: 0.027 gHa/cm2. signed rank p-value of 0.0004) at the end of the 6-month treatment with CP601B. These represented increases of approximately 2% in BMD for each of these areas.

TABLE 15
Bone Mineral Density Changes and Percent Changes From Baseline to Day 182
for Lumbar Spine and Hip - Subjects Naive to Previous Testosterone Replacement Products: Day
42/56 MITT Population

| | | | Final Dose Group^{a} | | | |
|---|---|---|---|---|---|---|
| | | | 2 g gel | 3 g gel | 4 g gel | |
| | | Statistic | (40 mg T)^{b} | (60 mg T)^{b} | (80 mg T)^{b} | All Doses |
| Hip | Change (gHa/cm²) | N | 4 | 11 | 11 | 26 |
| | From Baseline | Mean ± SD | 0.010 ± 0.012 | 0.037 ± 0.055 | 0.007 ± 0.059 | 0.020 ± 0.053 |
| | | Median | 0.014 | 0.027 | 0.021 | 0.020 |
| | | Range | −0.008-0.020 | −0.015-0.159 | −0.128-0.082 | −0.128-0.159 |
| | | p-value^{c} | | | | 0.0161 |
| | Percent Change From | N | 4 | 11 | 11 | 26 |
| | Baseline | Mean ± SD | 0.940 ± 1.410 | 3.365 ± 5.345 | 0.490 ± 5.556 | 1.776 ± 5.096 |
| | | Median | 1.415 | 2.535 | 2.130 | 2.000 |
| | | Range | −1.10-2.028 | −1.52-16.38 | −12.5-6.710 | −12.5-16.38 |
| | | p-value^{c} | | | | P = 0.0182 |
| Spine | Change (gHa/cm²) | N | 4 | 12 | 11 | 27 |
| | From Baseline | Mean ± SD | 0.036 ± 0.041 | 0.031 ± 0.052 | 0.020 ± 0.029 | 0.027 ± 0.041 |
| | | Median | 0.023 | 0.032 | 0.019 | 0.027 |
| | | Range | 0.002-0.095 | −0.074-0.123 | −0.029-0.072 | −0.074-0.123 |
| | | p-value^{c} | | | | 0.0004 |
| | Percent Change From | N | 4 | 12 | 11 | 27 |
| | Baseline | Mean ± SD | 4.017 ± 4.285 | 3.038 ± 4.314 | 1.689 ± 2.666 | 2.634 ± 3.671 |
| | | Median | 2.912 | 2.676 | 1.983 | 2.324 |
| | | Range | 0.235-10.01 | −4.13-10.93 | −3.32-5.931 | −4.13-10.93 |
| | | p-value^{c} | | | | 0.0006 |

^{a}Subjects in the 2 g and 3 g groups received 3 g daily only for the first 28 days; subjects in the 3 g group received 3 g daily for all 182 days.
^{b}For each gram of gel, 20 mg testosterone was applied to the skin.
^{c}Wilcoxon signed ranked test

Examination of BMD changes on an individual basis revealed that 65% (17/26) of the Day 42/56 MITT subjects had increases in hip BMD and 81% (22/27) had increases in spine BMD. The effect on BMD was similar for the Day 42/56 EE (N=17) and for the ITT (N=28) populations. The mean PK parameters observed in these subject subsets were similar to those observed in the main population.

Comparison of Results in Subpopulations

Rates of success for the study primary and secondary endpoints were computed for three subgroups of the main study population: subjects with no measurable endogenous testosterone, subjects with high BMI, and subjects younger than 55 or 55 years old and older. In addition, in each case, CP601B pharmacokinetics was further characterized and compared to that in the main study population.

Subjects With No Detectable Pre-treatment Testosterone

A number of subjects were enrolled in the study with no detectable pretreatment testosterone concentration (Day 1 C₀<50 ng/dL). This subset of subjects is worth noting because it is one of the most challenging populations to treat.

Summary statistics for Cₘᵢₙ, C_{avg}, and Cₘₐₓ of the subset of subjects with Day 1 C₀ below the limit of detection of the testosterone assay are shown in Table 16.

TABLE 16
Summary Statistics on Cₘᵢₙ, C_{avg}, and C_{max (ng/dL) in the}
Subset of Subjects with No Measurable Endogenouse
Testosterone at Start of Study^{a}: Day 42/56 MITT population

| | | Day 1 | Day 14 | Day 42/56 | Day 182 |
|---|---|---|---|---|---|
| | | (N = 15) | (N = 15) | (N = 15) | (N = 13) |
| Cₘᵢₙ | Mean | 25 (0.0) | 292 (148.3) | 267 (73.9) | 265 (111.2) |
| | (SD) | | | | |
| | Median | 25 | 243 | 255 | 275 |
| | Range | 25-25 | 103-646 | 174-435 | 25-423 |
| C_{avg} | Mean | 274 (177.9) | 620 (242.0) | 536 (203.7) | 490 (216.9) |
| | (SD) | | | | |
| | Median | 226 | 593 | 506 | 398 |
| | Range | 68-651 | 324-1172 | 265-909 | 280-1024 |
| Cₘₐₓ | Mean | 566 (392.6) | 1425 (824.3) | 1346 (825.5) | 920 (476.7) |
| | (SD) | | | | |
| | Median | 488 | 1127 | 1178 | 830 |
| | Range | 131-1203 | 683-3829 | 384-3247 | 395-1976 |

^{a}Subjects with no measurable endogenous testosterone at the start of study were the subjects with a Day 1 hr 0 testosterone concentration (Day 1 C₀) below the limit of quantitation (LOQ) of the testosterone assay (<50 ng/dL; assigned to 25 ng/dL for data analysis).

As expected from the definition of this subset, Cₘᵢₙ on Day 1 is the same for all subjects and entered as a value of 25 ng/dL [half way between 0 and 50 ng/dL, the limit of quantitation (LOQ) of the testosterone assay]. By Day 14, the mean Cₘᵢₙ increased sharply (by 267 ng/dL) to 292 ng/dL.

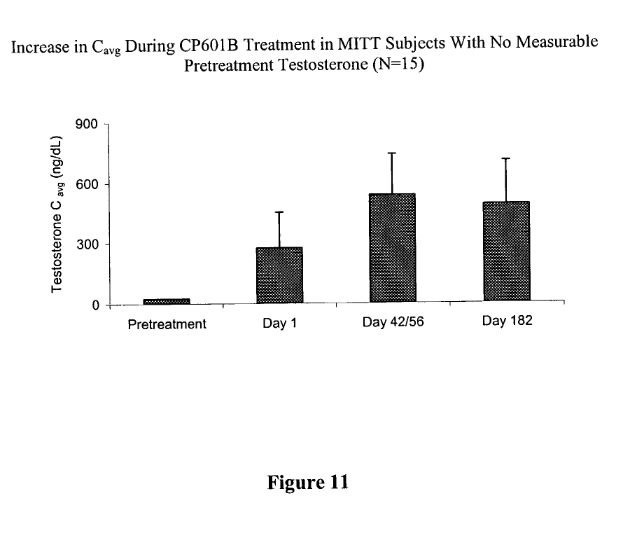

The increase in C_{avg} over time is shown in FIG. 11. Following a single application of 3 g of CP601B (Day 1), the mean C_{avg} value for these subjects was already increased to 274 ng/dL (up 249 ng/dL from 25 ng/dL). After steady-state was reached and the dose was adjusted properly in each subject, mean C_{avg} of 536 and 490 ng/dL were observed at Days 42/56 and 182, respectively, i.e., an additional increment of 262 ng/dL from Day 1, or a net increment of 490 ng/dL over pretreatment concentrations on Day 182.

Additionally, with respect to the study primary and secondary endpoints, results in this challenging population were comparable to results obtained with the main Day 42/56 EE and MITT populations. Seven of the 15 (47%) Day 42/56 MITT subjects with no measurable endogenous testosterone had both Cₘᵢₙ and C_{avg} within the PR and 14 subjects (93%) had C_{avg} within the PR; five of the 10 (50%) Day 42/56 EE subjects with no measurable endogenous testosterone had both Cₘᵢₙ and C_{avg} within the PR and nine subjects (90%) had C_{avg} within the PR.

It is interesting to note that although these men all had similar, non-measurable, pretreatment serum concentrations of testosterone, they were evenly distributed across the three dose groups after Day 28. Of the 15 men in the MITT subgroup, four men were assigned to the 2 g group, five men to the 4 g group and six men remained on the,3 g dose. Similar even distribution was observed in the EE population. This highlights the necessity of dose adjustment based on individual parameters beyond baseline testosterone concentrations, including individual skin permeability, clearance, body weight, and other factors. Furthermore, it demonstrates once more that the pretreatment testosterone concentration is not a good predictor of the final dose required for testosterone replacement therapy.

In summary, CP601B can effectively treat subjects with no measurable endogenous testosterone, one of the more challenging populations in testosterone replacement therapy. Increases in testosterone concentration (both in terms of Cₘᵢₙ and C_{avg}) were highly significant as early as Day 1 with only 3 g of gel. By the time steady-state was reached and the dose was properly adjusted, C_{avg} of about 500 ng/dL was achieved in these subjects. There is no indication that a larger amount of CP601B was required in this subpopulation. Additionally, rates of success on the primary and secondary endpoints in this challenging population and in the general population were similar, indicating that CP601B therapy can successfully treat all degrees of hypogonadism.

Subjects With High BMI

In some embodiments of the present invention, the subjects weigh above about 200 or 250 pounds for women, and 250 or 300 pounds for men and have their initial dosages set based upon their gender and body weight or BMI. In this study, no limitations on BMI were stated in the inclusion/exclusion criteria and subjects of diverse body weight and BMI were enrolled making it possible to assess the relationship between body weight or BMI and blood levels of testosterone as well between body weight or BMI and the efficacy of testosterone efficacy. The enrolled subjects' BMIs ranged from 16.5 to 54.5 kg/m². To determine if CP601B was effective in maintaining testosterone concentrations of morbidly obese hypogonadal men within the PR, the success rate on the primary and secondary study endpoints was calculated in 33 subjects with a BMI≧36 kg/M² and available data on Day 42/56 for Cₘᵢₙ and C_{avg}. Twelve of these 33 subjects (36%) had both Cₘᵢₙ and C_{avg} within the physiologic range. Additionally, of the 33 subjects, nine subjects had BMI≧45 kg/M². In this particularly challenging group, the success rate on the study primary endpoint remained high, with a value of 33%. Thus, the success rate on the study primary endpoint in high BMI subjects (BMI≧36 kg/m² and BMI≧45 kg/m²) was similar to that observed in the main ITT study population (34%).

Similar conclusions were reached on the secondary endpoint. Twenty-nine of the 33 subjects with BMI≧36 kg/m² (88%) had C_{avg} within the PR. In the subgroup of nine subjects with BMI≧45 kg/m², the success rate was 78%. Both these rates compared favorably with the 74.6% rate observed in the main ITT population (Table 14).

In summary, in this study, hypogonadal subjects with a wide range of BMI were enrolled, including a significant number with BMI≧36 kg/m². On Day 42/56 in 33 subjects with BMI≧36 kg/m² and in a subset of nine subjects with BMI≧45 kg/m², the success rates of primary and secondary endpoints were similar to those observed in the main ITT population. These results support the conclusion that CP601B can effectively treat morbidly obese hypogonadal men.

Effect of Age

The correlation of Cₘᵢₙ, C_{avg}, and Cₘₐₓ with age was studied on Day 14 and on Day 42/56. Results (not otherwise shown) showed that there was no correlation between age and any of the three PK parameters at any of the two days.

The effect of age on the primary and secondary endpoint results was also assessed. About 45% of the 71 Day 42/56 MITT subjects who were 55 or older had both C_{avg} and Cₘᵢₙ values within the PR on Day 42/56 (primary endpoint), compared with 39.1% of the 92 subjects who were younger than 55. The percentage of subjects with C_{avg} values within the PR was high for both population subgroups (older subjects [94.4%] and younger subjects [90.2%]). The two age groups were comparable for other efficacy assessments (percentage of subjects with C_{avg} values within the PR and testosterone concentrations ≧300 ng/dL for at least 80% of the dosing interval, and percentage of subjects with testosterone concentrations within the PR for at least 80% of the dosing interval).

Decrease of Testosterone Concentrations Following CP601B Therapy Discontinuation (Amendment #5)

To determine the rate of washout following CP601B therapy discontinuation, serum testosterone concentrations were measured immediately before (Day 182 hr 0) and 24, 48, 72, and 96 hours following application of the last dose of CP601B. Analysis was performed on samples from five subjects and testosterone concentrations were compared to their testosterone level at entry in the study, Day 1 C₀. Two of the five subjects returned to baseline levels in less than 48 hours; two others, in less than 72 hours. One subject with no measurable testosterone at baseline had a testosterone concentration of 171 ng/dL at 96-hour postdose. In general, these data indicate that in most subjects testosterone concentration return to baseline in 2-3 days following treatment discontinuation.

PK Summary and Conclusions

The following points summarize the key pharmacokinetic findings:

The average testosterone concentration (±SD) on entry into study was 181.0±88.6 ng/dL for the Day 42/56 EE population and 202.3±121.7 ng/dL for the Day 42/56 MITT population. These levels were similar to that observed in the ITT population (204.0±118.68 ng/dL).

Twenty-four hour pretreatment testosterone concentration-time profiles were obtained in 10 subjects. The mean pretreatment profile was well below 300 ng/dL, with a mean C_{avg} of 175.8±91.8 ng/dL. There were no overall discernible diurnal variations.

After only one dose of CP601B had been applied (3 g gel on Day 1), the mean C_{avg} for the 163 Day 42/56 MITT subjects reached 360.3±155.6 ng/dL, an average increase of 158 ng/dL from the testosterone concentration on entry. In the Day 42/56 EE population, this increase was slightly higher, with an average increase of about 200 ng/dL from the testosterone concentration on entry.

Analysis of mean testosterone concentration-time curves showed that, following 14 days of treatment with a fixed dose of CP601B (3 g), subjects could be individually assessed and assigned to one of three groups depending on whether their dose of CP601B was to be increased to ⁴ g, decreased to 2 g, or kept the same. Following dose adjustment, the mean testosterone concentration-time curves were almost superimposable for all three groups at both Days 42/56 and 182.

The inter- and intraindividual CVs were calculated in the 3 g group. The interindividual CV for C_{avg} was about 30% and the intraindividual CV was about 22% in both the Day 42/56 EE and MITT populations.

In the three final dose groups, the change in CP601B dose correlated with expected and highly significant changes in Cₘᵢₙ, C_{avg}, and Cₘₐₓ. There were large differences in all three PK parameters, particularly C_{avg} and Cₘₐₓ on Day 14 among the three final dose groups, which disappeared following dose adjustment. Dose adjustment had a highly significant effect in decreasing testosterone concentrations and the corresponding PK parameters that were judged as too high, or increasing those that were too low. Thus, dose adjustment is an effective means of individualizing therapy with CP601B. The results of the analysis of Cₘᵢₙ, C_{avg}, and Cₘₐₓ confirmed those obtained from the mean testosterone profiles.

A single daily dose of CP601B provides continuous testosterone replacement for hypogonadal men throughout the entire 24-hour dosing interval.

The primary endpoint (Cₘᵢₙ and C_{avg} within the PR) was met in all three study populations (EE, MITT and ITT) on both the primary (Day 42/56) and the secondary (Day 182) efficacy days. Specifically, in the Day 42/56 MITT population, 41.7% of subjects (68 of 163) had Day 42/56 C_{avg} and Cₘᵢₙ within the PR. The lower bound of the 95% CI (34.1%) was higher than the 20% non-inferiority margin for the 35% historic point estimate with an allowable delta of 15%.

The secondary endpoint (C_{avg} within the PR) was met in the Day 42/56 and Day 182 EE and MITT, and in the ITT population on Day 42/56. Specifically, in the Day 42/56 MITT population, 92% of subjects (150 of 163) had C_{avg} within the PR. The lower bound of the 95% CI (86.5%) was higher than the 65% non-inferiority margin for the 80% historic point estimate with an allowable delta of 15%.

CP601B can effectively treat subjects with no detectable endogenous testosterone, a challenging population in testosterone replacement therapy. Increases in testosterone concentration (both in terms of Cₘᵢₙ and C_{avg}) were highly significant as early as Day 1 with only 3 g of gel. By the time when steady-state was reached and the dose was properly adjusted in all subjects, C_{avg} of about 500 ng/dL was achieved. Additionally, rates of success on the primary and secondary endpoints in this population were similar to those in the general population, indicating that CP601B therapy can successfully treat all degrees of hypogonadism.

The relative systemic bioavailability with CP601B was estimated to be 12.2±4.6%.

Other hormones: Serum DHT concentrations increased from a mean of 18.5 ng/dL at baseline to 78.0 ng/dL on Day 14, and 86.4 ng/dL on Day 182 in the Day 42/56 MITT subjects. The DHT to testosterone ratio increased from 0.12 at baseline to 0.21 on Day 42/56 and 0.22 on Day 182 in the same group of men. DHT to testosterone ratios were thus numerically higher on Day 182 than at baseline, but these differences did not achieve statistical significance. Serum BAT levels increased in a similar fashion but remained within the normal range for males. Treatment with CP601B was associated with a significant increase in E₂ concentrations from a mean of 1.6 ng/dL to a plateau around 3.5 ng/dL throughout the rest of the study, in the upper end of the normal range for healthy young men. The mean estradiol to testosterone ratio remained unchanged throughout the study. Mean values for SHBG did not change significantly over the course of 182 days of treatment with CP601B. Levels of FSH and LH decreased modestly, as expected. Results in the Day 42/56 MITT population were similar to those in the Day 42/56 EE and ITT populations.

BMD: Following 6-month CP601B treatment, a statistically significant 2% increase in hip and spine BMD was observed in subjects who had never used testosterone replacement products.

Cₘᵢₙ, C_{avg}, and Cₘₐₓ were all highly and inversely correlated with BMI and weight before dose adjustment. Following dose adjustment, there was no correlation in the 2- and 3-g groups, but the correlation remained in the 4-g group.

CP601B can effectively treat morbidly obese hypogonadal men. Analysis of primary and secondary study endpoints on Day 42/56 in 33 subjects with BMI≧36 kg/m² and in a subset of nine subjects with BMI≧45 kg/m² showed that success rates in these challenging populations were similar to those observed in the main ITT population.

There was no correlation between age and any of the PK parameters. CP601B was equally effective in treating younger subjects (<55 years) and older subjects (≧55 years).

Washout data indicated that in most subjects testosterone concentration return to baseline in 2-3 days following treatment discontinuation

Since efficacy results in the EE and MITT populations were similar, it can be concluded that results obtained in the smaller EE population could be generalized to a less restrictively defined hypogonadal population, and that CP601B is a successful testosterone replacement therapy in this population.

A change in PK parameter values following a dose change is a welcome characteristic for a topical product, as it allows for more accurate and predictable dose adjustments by physicians. This property is not trivial, however, and is clearly formulation-dependent. For example, with AndroGel, a commercially available 1% testosterone transdermal gel, a 50% increase in dose (from 5 to 7.5 g gel per day) did not translate into any increase in mean C_{avg} (C_{avg} 5 g was 455 ng/dL whereas C_{avg} 7.5 g was 450 ng/dL, n=18-20).

Dose Adjustment With CP601B: Guidance for Physicians

The importance and effectiveness of dose adjustment with CP601B has been described above. Here, adjustment decisions were made based on a complete 24-hr testosterone PK profile (Day 14). Such bounty of data is not likely to be available to practitioners when deciding whether to change a patient's dose. This section describes how the data in the 24-hr PK profile can be effectively reduced to a single blood sample and proposes guidance for use by persons when adjusting the CP601B dose in hypogonadal subjects.

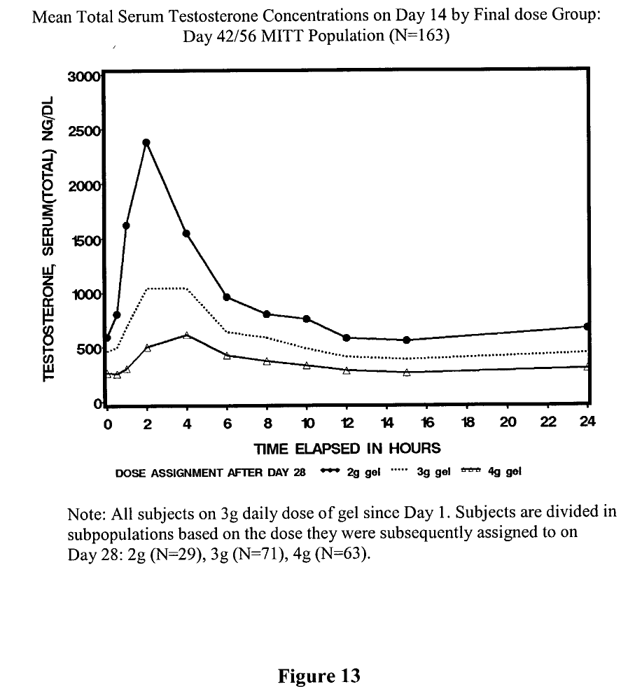

The time point provided the most spread between the three mean testosterone concentration time-profiles on Day 14 is the 2-hr time point (FIG. 13).

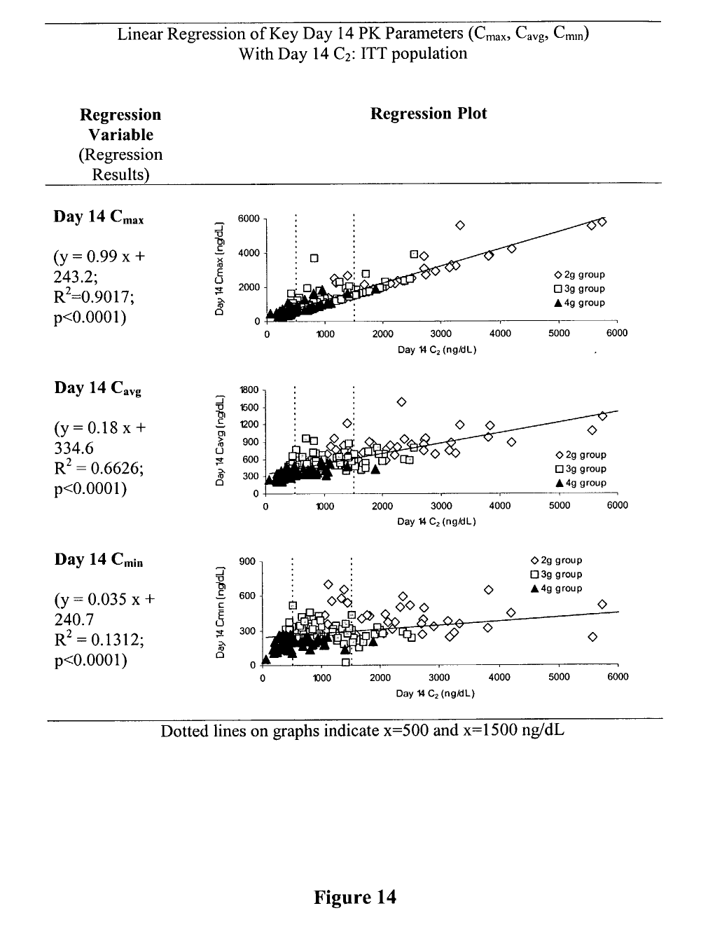

Based on these observations, the correlation between Day 14 C₂, the testosterone concentration measured at 2-hr after CP601B application on Day 14, and all three PK parameters on Day 14 was investigated. Results are shown in FIG. 14. Not surprisingly, there was a highly significant correlation between Day 14 C₂ and Day 14 Cₘₐₓ (R²>0.9). Interestingly, there was also a strong correlation with Day 14 C_{avg}. The correlation with Cₘᵢₙ was marginal.

These results indicate that Day 14 C₂ is a good surrogate marker for both Cₘₐₓ and C_{avg} on Day 14. Because C_{avg} is directly related to testosterone treatment efficacy (Bhasin S, Woodhouse L, Casaburi R, et al. Am J Physiol Endocrinol Metab 2001; 281:E1172-81) and Cₘₐₓ is a key parameter to consider to insure patient's safety during therapy, Day 14 C₂ is an optimal single time point to describe the full 24-hr PK profile on Day 14.

Additionally, based on the distribution of data points for each final dose group, the following guidance is recommended for subjects on a starting dose of 3 g gel (60 mg T applied to the skin) for at least 14 days:

If Day 14 C₂<500 ng/dL: Increase dose to 4 g gel

If Day 14 C2>1500 ng/dL: Decrease dose to 2 g gel

If Day 14 C2 500 to 1500 ng/dL: Keep dose at 3 g gel

Having established that Day 14 C₂ would be the optimal single time point to use in assessing a dose or dose change (per the guidance discussed above), dose assignment as conducted herein was compared to the dose assignment predicted from the guidance.

Within this study, subjects were included who had a wide range of BMI: 1 subject (0.5%) had a BMI under 18.5 (considered to be underweight by the standards set by the Mayo Clinic), 13 subjects (6.5%) had BMI between 18.5 and 24.9 (considered to be normal), 74 subjects (36.8%) had BMI between 25 and 29.9 (considered to be overweight), 95 subjects (47.3%) had BMI between 30 and 39.9 (considered to be obese), and 18 subjects (9.0%) had BMI greater than 40 (considered to be morbidly obese). Based on the correlation between BMI and dose required bring the testosterone levels of the subjects within the normal physiological range, the following guidance is recommended for estimating the correct starting dose for a given individual:

If BMI<18.5 (underweight): starting dose is 2 g gel.

If BMI is between 18.5 and 35 (normal to overweight): starting dose is 3 g gel.

If BMI>35 (obese or morbidly obese): starting dose is 4 g gel.

If the individual has a BMI between 18.5 and 35 (is normal to overweight), it is recommended that the Day 14 C₂ be determined and used to adjust the dose if necessary.

TABLE 17
Comparison of Dose Assignment by Various Guidance

| | | Day 42/56 | |
|---|---|---|---|
| | | Population | |
| Rule | Criterion | EE | MITT |
| | N | 89 | 163 |
| Day 1 rule | % of subjects directly assigned to their final | 40% | 44% |
| | dose by starting all subjects on 3 g CP601B | | |
| | on Day 1 | | |
| C₂ rule on | % of subjects assigned to the same final dose | 63% | 69% |
| Day 14^{a} | by using the Day 14 C₂ rule^{a} | | |
| | % of subjects grossly misadjusted by using | 0.0% | 0.0% |
| | the Day 14 C₂ rule^{b} | | |

^{a}If C₂ < 500 ng/dL: Increase dose to 4 g gel (80 mg T applied on the skin); If C₂ > 1500 ng/dL: Decrease dose to 2 g gel (40 mg T applied on the skin); If C₂ 500-1500 ng/dL: Keep dose at 3 g gel (60 mg T applied on the skin)
^{b}% of subjects adjusted to 4 g gel (80 mg T applied on the skin) per Day 14 C₂ rule whose final dose should have been 2 g gel (40 mg T applied on the skin) based on Study adjustment guidance, or vice versa.

As shown in Table 17, by assigning all subjects to a 3 g dose on Day 1, 42-44% of subjects can be assigned to their final dose as early as Day 1. When the Day 14 rule was tested against the criteria for dose adjustment on Day 14 set forth for the trial, using only one of the 12 time points constituting the full 24-hr PK profile on Day 14, about 70% of subjects could be adjusted to their final dose in the same manner as the present study. Because the Day 14 rule is conservative (on the side of safety), no patient is expected to be grossly overdosed by this rule (given 4 g gel instead of 2 g).

In conclusion, serum testosterone concentration is sensitive to dose adjustments using CP601B as reflected by the resultant changes in all key PK parameters following dosage adjustment. Guidance presented in this section that is easily implemented and can be used by physicians to guide dose adjustment during CP601B therapy in hypogonadal subjects.

Analysis of mean testosterone concentration-time curves showed that, following 14 days of treatment with a fixed dose of CP601B (3 g), subjects could be individually assessed and assigned to one of three groups depending on whether their dose of CP601B was to be increased to 4 g, decreased to 2 g, or kept the same. Following dose adjustment, the mean testosterone concentration-time curves were almost superimposable for all three groups at both Days 42/56 and 182. A single daily dose of CP601B provided testosterone replacement for hypogonadal men through the entire 24-hour dosing interval.

The large differences in all three PK parameters, particularly C_{avg} and Cₘₐₓ observed on Day 14 disappeared following dose adjustment. Dose adjustment had a highly significant effect in decreasing testosterone concentrations and the corresponding PK parameters that were judged as too high, or increasing those that were too low. Thus, an increase in dose for those subjects with testosterone concentrations below the PR and a decrease in dose for those with peak concentrations above this range is a means of individualizing therapy with CP601B. The study primary endpoint was met in all 3 study populations (EE, MITT and ITT). Specifically in the EE population, 41.6% [95% CI (31.3%, 51.8%)] of subjects had Day 42/56 C_{avg} and Cₘᵢₙ within the PR. The lower bound of the 95% CI was higher than the historical value selected (20%). A secondary endpoint (C_{avg} within the PR) was also met in the all three populations. Specifically, in the MITT population (n=163), 92% of subjects had C_{avg} within the PR. Efficacy results in the EE and MITT populations were similar, thus confirming that results obtained in the smaller EE population could be generalized to a less restrictively defined hypogonadal population.

CP601B can effectively treat subjects with no measurable endogenous testosterone or those in the morbidly obese BMI category (≧36). The rates of success on the primary and secondary endpoints in these challenging population subgroups were similar to those in the general population, indicating that CP601B therapy can successfully treat the entire hypogonadal population.

Serum DHT concentrations increased from a mean of 18.5 ng/dL at baseline to 78.0 ng/dL on Day 14, and 86.4 ng/dL on Day 182 in the Day 42/56 MITT subjects. The DHT to testosterone ratio increased from 0.12±0.15 at baseline to 0.21±0.09 on Day 42/56 and 0.22±0.10 on Day 182 in the same group of men. Thus, DHT to testosterone ratios were numerically higher on Day 182 than at baseline, but these differences did not achieve statistical significance. Serum BAT levels increased in a similar fashion but remained within the normal range for males. The mean estradiol to testosterone ratio remained unchanged throughout the study. Mean values for SHBG did not change significantly over the course of 182 days of treatment with CP601B. Levels of FSH and LH decreased modestly, as expected.

Following 6-months of CP601B treatment, a significant 2% increase in hip and spine BMD was observed in subjects who had never used testosterone replacement products.

In conclusion, single daily doses of CP601B raise and maintain serum testosterone levels into the physiologic range and provide adequate testosterone replacement for hypogonadal men. At steady state, a single 2-hour serum testosterone measurement after application of the CP601B gel on Day 14 provides physicians with the necessary information to appropriately adjust dose for each subject utilizing a simple dosage adjustment guidance. Additionally, using BMI alone, a starting dose can be estimated without knowledge of the initial serum testosterone measurement.

### Example 2

Patients suffering from chronic fatigue syndrome. may be treated with with 2 g, 3 g, or 4 g of a transdermal testserone gel containing a penetration enhancer. The dosage of testosterone given is based on the patient's BMI or body weight, The initial dose for treating the subjects will be determining according to the body weight and/or BMI of the subject; and then selecting the individual's dose according to a predetermined empirical relationship between the body weight or BMI, the applied dosage, and the serum level of the hormone in a reference population at steady state. For example, for an initial dose, patients with BMI's under 35 are given the 2 g dose on a daily basis; patients with BMI's between 35 and 45 are given 3 g dose on a daily basis; patients with BMI's above 45 are given the 4 g dose on a daily basis. The sex hormone(s) are administered in a semisolid topical gel formulation having a a pH value of between about 4 to about 8 and comprising 0.1% to about 2% w/w of each human sex hormone, a penetration-enhancer such s oleic oleic acid; and a C₁-C₄ alcohol; and a glycol. The patients note improvement in the following symptoms: short-term memory, concentration, throat soreness, lymph node tenderness, muscle pain, multi-joint pain, headache, sleep, and level of malaise.

### Example 3

Patients suffering from chronic Epstein-Barr virus infections are treated with 2 g, 3 g, or 4 g of a transdermal testosterone gel containing a penetration enhancer. The initial dose for treating the subjects will be determined according to the body weight and/or BMI of the subject; and then selecting the individual's dose according to a predetermined empirical relationship between the body weight or BMI, the applied dosage, and the serum level of the hormone in a reference population at steady state. For example, for an initial dose, patients with BMI's under 35 are given the 2 g dose on a daily basis; patients with BMI's between 35 and 45 are given the 3 g dose on a daily basis; patients with BMI's above 45 are given the 4 g dose on a daily basis. The sex hormone(s) are administered in a semisolid topical gel formulation having a a pH value of between about 4 to about 8 and comprising 0.1% to about 2% w/w of each human sex hormone, a penetration-enhancer such as oleic oleic acid; and a C₁-C₄ alcohol; and a glycol. The patients note improvement in the following symptoms: throat soreness, lymph node tenderness, muscle pain, and level of malaise. The use of the testosterone gel in these patients decreases the overall length and severity of the infection.

### Example 4

Young girls needing pubertal development or adults with Turners syndrome are treated with a semisolid topical composition comprising sex hormones. The hormones may be estrogen-like, progestin-like, androgen-like or a mixture thereof.an estrogen. For instance, the efficacy of hormone replacement for these patients can be evaluated using the following protocol.

Girls and women with Turner syndrome between the ages of 14 and 50 years can be administered hormone replacement therapy by semisolid topical gels. Three months before beginning treatment, all patients will discontinue use of all hormone replacement therapies. They will then be randomly assigned to one of two treatment groups to compare the effects of placebo gels or estrogen, progestin and testosterone gels on bone strength, muscle and fat mass and psychosocial well being. One group will use transdermal gels containing estrogen, progestin and testosterone on a daily basis, while the other group will use placebo gels. Neither study participants nor the doctors will know who is getting the hormones until the study is complete. Patients will undergo the following procedures before beginning treatment and at 6, 12 and 24 months after starting treatment:

Physical examination.

DEXA scans (dual energy X-ray absorptiometry) to measure body composition and bone thickness. Low radiation X-rays scan the whole body to measure fat, muscle and bone mineral content..

Magnetic resonance imaging (MRI) scan of the abdomen to measure the amount of fat around the internal organs. The patient lies on a stretcher in a large tube surrounded by a magnetic field during the scanning. The procedure uses a strong magnet and radio waves to produce the images.

Heel ultrasound to measure bone thickness. The heel is placed in a chamber and sound waves pass through it to produce images.

Oral glucose tolerance test (OGTT) for diabetes and problems with carbohydrate metabolism. The patient drinks a sugary substance. A small amount of blood is drawn before taking the drink and four times afterwards.

Blood and urine tests to measure blood counts, liver and kidney function, ovarian hormones, growth factors, thyroid function, blood lipids, bone strength markers, and to test for pregnancy.

Blood pressure measurements.

Psychological testing for the effect of treatment on mood, self-esteem, quality of life, social shyness, anxiety and sexual function.

Neurocognitive tests (at first inpatient visit and 1 and 2 years after starting treatment) to measure nonverbal memory and visual-perceptual abilities.

During the hospital admissions, patients will be given a “metabolic diet” that contains specific amounts of salt and carbohydrates to ensure accurate blood pressure and sugar metabolism measurements. Patients will keep a record of their menstrual periods and physical activity throughout the treatment period.

Inclusion Criteria:

Girls and women with TS diagnosed by karyotype or other genetic evidence of X-chromosome defects and ovarian failure (diagnosed by failure to enter puberty spontaneously by age 18 or 2nd degree amenorrhea greater than 6 months and FSH greater than 40 mIU/ml)

Subjects with TS who have been previously exposed to estrogen and progestin effect, either endogenous or exogenous by medical treatment, sufficient to establish secondary sexual development and menses

Subjects with TS—ages 14 to 50, who have completed near final height, as demonstrated by a bone age of greater than or equal to 14 years

Exclusion Criteria:

Chronological or bone age of less than 14 years

Chronological age greater than 50 years

Chromosomal disorders in addition to TS

Absence of 2nd degree sexual development

Growth hormone or androgen treatment within 6 months of starting study.

Testosterone level greater than normal range for age.

Contraindications to the use of estrogen, progestin or androgens: Neoplasia; Hypercoagulation disorder; Pregnancy; Gall bladder, biliary or liver parenchymal disease (evidenced by jaundice, gastrointestinal symptomatology, other clinical evidence of cholelithiasis or hepatitis); Hypertriglyceridemia (TGs greater than 300); Active coronary disease (evidenced by documented MI or coronary angiography.

Mental or physical disability, which in the estimation of study investigators, prevents a candidate from participation in study.

The initial dose for treating the subjects will be determined according to the body weight and/or BMI of the subject; and then selecting the individual's dose according to a predetermined empirical relationship between the body weight or BMI, the applied dosage, and the serum level of the hormone in a reference population at steady state. The sex hormone(s) are administered in a semisolid topical gel formulation having a a pH value of between about 4 to about 8 and comprising 0.1% to about 2% w/w of each human sex hormone, a penetration-enhancer such s oleic oleic acid; and a C₁-C₄ alcohol; and a glycol.

### Example 5

The subjects to be administered the testosterone gel composition are female subjects with signs and/or symptoms of sexual dysfunction. Such subjects are identified by completing the FSFI questionnaire to confirm the diagnosis of FSD (Rosen R et al.: The Female Sexual Function Index (FSFI): A Multi-dimentional Self-report Instrument for the Assessment of Female Sexual Function. J Sex and Marital Therapy, 26:191-208, 2000).

Such subjects will be considered distressed if the their Female Sexual Distress Scale is equal or larger than 15 (Derogatis L.: Development of the Female Sexual Distress Scale (FSDS): Preliminary Study. Presented at Female Sexual Function Forum Meeting at Boston, Mass., Oct. 22-26, 2000). Upon identification, the serum testosterone concentration, both total, bioactive or free testosterone levels of the female subjects scan be determined to assess whether the subject is testosterone deficient. If the results of the hormone analyses show evidence of testosterone deficiency, subjects will benefit from Androgen replacement therapy. One treatment objective for these subjects would be to bring the testosterone level into the normal physiologic range, preferably into the upper one-third of the physiologic range, for example into 8-14.5 ng/dl of bioactive testosterone concentration range.

To determine the optimal starting dose, subject's BMI is calculated based on kg/m². Once BMI is determined, subjects with a low BMI would start on a lower dose of hormone replacement or androgen replacement therapy, and subjects with a high BMI would be started on a higher intial dose of androgen replacement therapy.

### Example 6

For male subjects reporting some form of sexual dysfunction, subjects could first complete the ADAM questionnaire (Morley J E, Charlton E, Patrick P, Kaiser F E, Cadeau P, McCready D, Perry H M 3rd.Validation of a screening questionnaire for androgen deficiency in aging males. Metabolism. 2000 September;49(9):1239-42.) which provides evidence of testosterone deficiency in males before blood testosterone analyses. Once the ADAM questionaire indicates an androgen deficiency in these subjects, blood testosterone concentration can be measured and compared with known physiologic range. Depending on the assay used, the normal range for serum testosterone levels in early morning hours in healthy, young men, 20-40 years of age, is approximately 300-1200 ng/dl. If the serum testosterone is below 300 ng/dl, the subject is qualified for testosterone replacement therapy. Since the serum SHGB (sex hormone binding globulin) level increases with age, a more specific method to diagnosed testosterone deficiency is measured either the bioactive testosterone concentration or look at the total testosterone/SHBG (T/SHBG) ratio.

To determine the optimal starting dose, subject's BMI is calculated based on kg/m². Once BMI is determined, subjects with a low BMI would start on a lower dose of hormone therapy or androgen therapy, and subjects with a high BMI would be started on a higher intial dose of androgen replacement therapy and be administered a semisolid topical semisolid gels comprising a therapeutic amount of the hormone and an effective amount of the penetration enhancer.

### Example 7

This example illustrates the use of the compositions and methods of the invention to treat androgen deficiency states in females.

Androgen insufficiency in women occurs for several reasons. Recognized causes include hypopituitarism, Addison's disease, corticosteroid therapy, chronic illness such as diabetes, cancer, AIDS, etc, ovarian failure, oophorectomy, oral estorogen therapy or oral contraceptive use. In fact, an international consensus conference convened in 2001 defined the condition based on available evidence and provided a consensus paper on the definition, classification and assessment (Bachman G et al, “Female Androgen Insufficiency: The Princeton Consensus Statement on Definition, Classification, and Assessment. Fertility and Sterility, 77(4)660-665, 2002).

Post-menopausal women distressed with sexual dysfunction, depression, lost of energy, reduction in the overall sense of well-being were recruited in this study. Plasma levels of total and bioactive testosterone were measured to verify that these women suffered from “Female Androgen Insufficiency” syndrome.

Women participated in this study were randomly exposed to 2 mg, 4 mg, and 8 mg testosterone delivered from the 1% testosterone gel, the BMI correlation was conducted based on total and bioactive testosterone levels using a linear regression model.

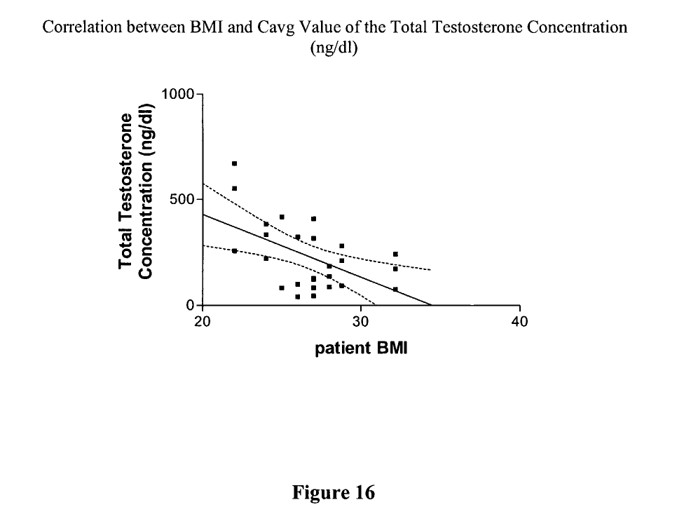

Results showed in FIG. 16 indicate that in post-menopausal women treated with the topical 1% testosterone gel, a consistent negative correlation between Cₘₐₓ, C_{avg} of the total testosterone and Cₘₐₓ of bioactive testosterone and BMI. Statistic significance of the analyses is shown in the Table below. As a result, once women came in to physician's office and are diagnosed as having “Androgen Insufficiency”, based on the BMI of each women's BMI value, proper dose of testosterone can be administered to replace the serum testosterone into upper one-third of the physiological range and therapeutic efficacy will ensue.

Statistic Analyses of the Significance of the Correlation of Various Pharmacokinetic Parameters and BMI

| | Statistic Significance with BMI |
|---|---|
| Cmax of Total Testosterone conc. | P = 0.006 |
| Cavg of Total Testosterone conc. | P = 0.006 |
| Cmax of the Biotestosterone conc. | P = 0.097 |

### Example 8

To evaluate the effect of CP601B on bone mineral density (BMD) a subset of subjects from Example 1 who had not had previous androgen therapy and were enrolled at a site where the investigator had the required equipment. Each subject was evaluated before and after 6 months of continuous treatment. In the 27 subjects tested, bone mineral density increased 1.78% in the hip and 2.63% in the spine. The increase in BMD associated with replacement testosterone treatment with CP601B compares, even after a relatively short treatment duration of 6-months, very favorably to other androgen products on the market.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for determining the initial dose to administer to a human subject of a semisolid topical composition having a pH value of between about 4 to about 8 and comprising 0.1% to about 2% w/w of a human sex hormone, a penetration-enhancing amount of oleic acid; a C₁-C₄ alcohol; and a glycol, said method comprising:
determining the body weight of the subject;
selecting the dose according to a predetermined empirical relationship between the body weight, the applied dosage, and the serum level of the hormone in a reference population at steady state.

2. The method of claim 1, wherein the relationship is between BMI, the applied dosage, and the C_{avg} serum level of the hormone.

3. The method of claim 1, wherein the hormone is testosterone.

4. The method of claim 1, wherein the hormone is estrogen.

5 The method of claim 1, wherein the serum level is Cₘᵢₙ.

6. The method of claim 1, wherein if the subject has a BMI less than 18.5, the initial dose is 2 g gel; if the subject has a BMI is between 18.5 and 35, the initial dose is 3 g gel; or if the subject has a BMI greater than 35, the initial dose is 4 g gel.

7. A method for determining the dose to administer to a human subject of a semisolid topical composition comprising a therapeutic amount of a mammalian hormone and an effective amount of a penetration enhancer, said method comprising:
determining the weight and height of the subject;
calculating the Body Mass Index (BMI) of the subject;
and adjusting the dose according to the BMI.

8. The method of claim 7 wherein the adjusting is based upon a predetermined empirical relationship between BMI, the amount of the composition applied, and the serum hormone concentration level measured in a reference population at steady state.

9. The method of claim 7, wherein the hormone is a human sex hormone.

10. The method of claim 7, wherein the hormone is an androgen.

11. The method of claim 9, wherein the hormone is an estrogen.

12. The method of claim 10, wherein the hormone is testosterone or a salt or ester thereof.

13. The method of claim 11, wherein the hormone is a pharmaceutically acceptable salt or ester of estrogen or estradiol.

14. The method of claim 7, wherein the hormone is a progestin.

15. The method of claim 14, wherein the hormone is progesterone or a salt or ester of progesterone.

16. The method of claim 7, wherein the enhancer is oleic acid.

17. The method of claim 7, wherein the hormone has a steroid moeity.

18. The method of claim 7, wherein the topical composition has a pH value of between about 4 to about 8 and comprises
a) the hormone in a concentration of about 0.1% to about 2% w/w, and
b) a penetration-enhancing system consisting essentially of (i) a membrane fluidizer comprising oleic acid; (ii) a C₁-C₄ alcohol; and (iii) a glycol.

19. The method of claim 17, wherein the hormone is a member selected from the group consisting of testosterone, estradiol, progestin, and derivatives and mixtures thereof.

20. The method of claim 18, wherein the penetration-enhancing system further comprises (iv) a gelling agent.

21. The method of claim 10, wherein the subject is a male with primary or secondary hypogonadism, male sexual desire disorder, male sexual arousal disorder, AIDS, wasting syndrome associated with chronic illnesses, end stage renal disease, chronic fatigue syndrome, Epstein-Barr virus, heart disease, cancer, diabetes, Alzheimer's disease, systemic lupus erythematosus, rheumatoid arthritis, multiple sclerosis, or osteoporosis.

22. The method of claim 7, wherein the subject is a female with sexual dysfunction or with a reduced feeling of well-being, AIDS, wasting syndrome associated with chronic illnesses, end stage renal disease, chronic fatigue syndrome, Epstein-Barr virus, heart disease, cancer, diabetes, Alzheimer's disease, systemic lupus erythematosus, rheumatoid arthritis, multiple sclerosis, osteoporosis, or Turner's Syndrome.

23. The method of claim 8, wherein adjusting the dose increases the dose by an average of about 1-10% for each single unit increase in the BMI for subjects having a BMI value of 30 to 50 kg/m².

24. The method of claim 10, wherein the subject is male and had a pretreatment average serum testosterone level below 300 ng/dl.

25. The method of claim 7, wherein the dose is an initial dose.

26. The method of claim 7, wherein said composition is selected from the group consisting of solutions, creams, lotions, ointments, and gels.

27. The method of claim 26, wherein said composition has a testosterone concentration of about 0.01% to about 5% w/w.

28. The method of claim 7, wherein the dose is a daily dosage.

29. A method of administering a therapeutically effective amount of a mammalian sex hormone to a human subject; said method comprising:
calculating a dose of a topical composition comprising the sex hormone and a penetration enhancer according to the method of claim 1; and
administering the dose of the composition to the skin of the subject.

30. A method of restoring serum sex hormone levels in a human subject to normal levels, said method comprising:
determining the height and weight of the subject;
using the height and weight of the subject to estimate a first dose amount of a topical composition comprising the sex hormone and a penetration enhancer;
applying at least daily to the skin of the subject the composition in the first dose amount;
measuring the level of the sex hormone in the blood of the subject; and
if the blood sex hormone level is below a first predetermined level, treating the subject with a second dose 25 to 100% greater than the first dose amount; or, if the blood sex hormone level is above or near a second predetermined level, treating the subject with a third dose which is 25 to 75% less than the first dose amount

31. The method of claim 30, wherein the sex hormone is an androgen selected from the group consisting of testosterone, its salts, esters, and derivatives.

32. The method of claim 31, wherein the testosterone is measured two hours after the initial daily application of the first dose amount.

33. The method of claim 31, wherein the testosterone is measured at the blood testosterone steady state.

34. The method of claim 30, wherein the testosterone is measured at least three days after the first application of the composition.

35. The method of claim 30, wherein the topical composition has a pH value of between about 4 to about 8, and comprises:
a) the hormone in a concentration of about 0.1% to about 2% w/w, and
b) a penetration-enhancing system consisting essentially of (i) a membrane fluidizer comprising oleic acid; (ii) a C₁-C₄ alcohol; and (iii) a glycol.

36. The method of claim 30, wherein the sex hormone is an androgen or an estrogen.

37. The method of claim 33, wherein the first predetermined level is about 250-350 ng/dl and the second predetermined level is about 1000-1200 ng/dl.

38. A metered device for delivering a topical composition comprising testosterone and a penetration enhancer to a subject, wherein said metered device provides an identical amount of the composition on each administration and wherein the amount administered is determined by the BMI of the subject.

39. The kit comprising:
a pharmaceutical composition comprising a therapeutic amount of a mammalian hormone and an effective amount of a skin penetration enhancer;
a metered dose pump holding said composition in an amount to provide more than one dose; said pump set to deliver a fixed amount of the pharmaceutical composition when the pump is activated.

40. The kit of claim 39 further comprising instructions on how to activate the pump.

41. The kit of claim 39 further comprising instructions on where to apply the composition to the skin.

42. A method for determining the dose to administer to a human subject of a semisolid topical composition comprising a therapeutic amount of a mammalian hormone and an effective amount of a penetration enhancer, said method comprising:
determining the body weight and height of the subject;
and adjusting the dose according to the body weight.

43. The method of claim 42, wherein the adjusting is based upon a predetermined empirical relationship between body weight, the amount of the composition applied, and the serum hormone concentration level measured in a reference population at steady state.

44. A method of adjusting serum sex hormone levels in a human subject to normal levels, said method comprising:
determining the weight of the subject;
using the weight of the subject to establish a first dose amount of a topical composition comprising the sex hormone and a penetration enhancer wherein the establishing is based upon a predetermined empirical relationship between body weight, the amount of the composition applied, and the serum hormone concentration level measured in a reference population at steady state;
applying at least daily to the skin of the subject the composition in the first dose amount;
measuring the level of the sex hormone in the blood of the subject; and
if the blood sex hormone level is below a first predetermined level treating the subject with a second dose 25 to 100% greater than the first dose amount; or if the blood sex hormone level is above or near a second predetermined level, treating the subject with a third dose which is 25 to 75% less than the first dose amount.
